(19) United States
(12) Patent Application Publication
(10) Pub. No.: US 2021/0163929 A1
(43) Pub. Date: Jun. 3, 2021

(54) METHODS AND COMPOSITIONS FOR THE TREATMENT OF HEPATIC AND METABOLIC DISEASES

(71) Applicant: The Regents of the University of California, Oakland, CA (US)

(72) Inventor: Yu-Jui Yvonne Wan, Davis, CA (US)

(73) Assignee: The Regents of the University of California, Oakland, CA (US)

(21) Appl. No.: 17/093,225

(22) Filed: Nov. 9, 2020

Related U.S. Application Data
(63) Continuation of application No. PCT/US2019/031847, filed on May 10, 2019.
(60) Provisional application No. 62/670,559, filed on May 11, 2018.

(51) Int. Cl. C12N 15/11; A61K 47/46; A61K 47/36; A61K 47/40; A61K 47/32; A61K 38/18; A61K 31/203; A61K 31/155; A61K 31/575; A61K 31/718; A61K 45/06; A61P 1/16; A61P 3/06; A61P 3/10; A61K 35/761; A61P 3/04; A61K 31/22

(52) U.S. Cl. CPC C12N 15/11; C12N 2310/141; A61K 47/36; A61K 47/40; A61K 47/32; A61K 38/1825; A61K 31/203; A61K 31/155; A61K 31/575; A61K 31/718; A61K 45/06; A61P 1/16; A61P 3/06; A61P 3/10; A61K 35/761; A61P 3/04; A61K 31/22; C12N 2310/13; A61K 47/46

(57) ABSTRACT

The invention provides methods and pharmaceutical compositions for treating a metabolic disease in a subject. In some aspects, the invention comprises administering to the subject a therapeutically effective amount of a miR-22 inhibitor. In some embodiments, the invention further comprises administering to the subject a metabolism-enhancing agent such as a miR-22 inducer.

### CROSS-REFERENCE TO RELATED APPLICATIONS

This application is a continuation of International Application No. PCT/US2019/031847, filed May 10, 2019, which claims priority to U.S. Provisional Application No. 62/670,559, filed May 11, 2018, the disclosures of which are hereby incorporated by reference in their entirety for all purposes.

### SEQUENCE LISTING

The instant application contains a Sequence Listing which has been submitted electronically in ASCII format and is hereby incorporated by reference in its entirety. Said ASCII copy, created May 30, 2019, is named SL 070772-225410US-1218432 and is 1,311 bytes in size.

### BACKGROUND OF THE INVENTION

Metabolic diseases such as diabetes, obesity, non-alcoholic steatohepatitis (NASH) and non-alcoholic fatty liver disease (NAFLD) pose prominent threats to health worldwide, and are expected to continue to become more prominent. In 2015, nearly 10% of the American population had diabetes. In addition, more than one-third of American adults have obesity.

Accordingly, there is a need for new treatments and preventive measures for metabolic diseases such as diabetes, obesity, and fatty liver syndromes. The present invention satisfies this need, and provides related advantages as well.

### BRIEF SUMMARY OF THE INVENTION

In one aspect, the present invention provides a method for preventing or treating a metabolic disease in a subject. In some embodiments, the method comprises administering to the subject a therapeutically effective amount of a microRNA-22 (miR-22) inhibitor. In some embodiments, the miR-22 inhibitor is an inhibitor of human miR-22 (hsa-miR-22).

In some embodiments, the miR-22 inhibitor is an oligonucleotide. In some embodiments, the oligonucleotide comprises a nucleic acid sequence that hybridizes to miR-22 and reduces miR-22 expression. In some embodiments, the oligonucleotide comprises unmodified and/or modified nucleotides. In some embodiments, the oligonucleotide comprises a nucleic acid sequence that has at least about 80% sequence identity to SEQ ID NO:1. In some embodiments, the oligonucleotide comprises the nucleic acid sequence set forth in SEQ ID NO:1. In some embodiments, the oligonucleotide is virally expressed. In some embodiments, the oligonucleotide is expressed from an adenovirus, an adeno-associated virus (AAV), or a lentivirus. In some embodiments, the oligonucleotide is delivered using a non-viral delivery system. In some embodiments, a miR-22 inhibitor is a small molecule compound that binds to miR-22 and decreases or abolishes its activity.

In some embodiments, the method further comprises administering to the subject a therapeutically effective amount of a metabolism-enhancing agent. In some embodiments, the metabolism-enhancing agent induces miR-22. In some embodiments, the metabolism-enhancing agent is selected from the group consisting of fibroblast growth factor 21 (FGF21), an FGF21 mimic, an FGF21 inducer, a retinoid, a histone deacetylase (HDAC) inhibitor, metformin, a bile acid or an analog thereof, a resistant starch, a prebiotic agent, a probiotic agent, and a combination thereof. In some embodiments, the retinoid is selected from the group consisting of retinoic acid (RA), retinol, retinal, isotretinoin, alltretinoin, etretinate, acitretin, tazarotene, bexarotene, adapalene, seletinoid G, a retinyl ester, fenretinide, derivatives thereof, and a combination thereof. In some embodiments, the retinyl ester is selected from the group consisting of retinyl acetate, retinyl butyrate, retinyl propionate, retinyl palmitate, and a combination thereof.

In some embodiments, the HDAC inhibitor is selected from the group consisting of a short-chain fatty acid (SCFA), suberanilohydroxamic acid (SAHA), trichostatin, and a combination thereof. In some embodiments, the SCFA is selected from the group consisting of propionate, butyrate, isobutyrate, valerate, isovalerate, and a combination thereof. In some embodiments, the SCFA is butyrate.

In some embodiments, the bile acid or analog thereof comprises obeticholic acid, an agonist for bile acid receptors including FXR (farnesoid×receptor) and TGR5 (G protein-coupled bile acid receptor, also known as GPBAR1). In some embodiments, the probiotic comprises a bacterium that produces an SCFA. In some embodiments, the prebiotic comprises apple pectin, an inulin, or a combination thereof. In some embodiments, the metabolism-enhancing agent is administered orally. In some embodiments, the FGF21 is a virally or bacterially expressed recombinant protein. In some embodiments, the FGF21 is expressed from an adenovirus. In some embodiments, the FGF21 is a FGF21 mimic that increases FGF21 activity.

In some embodiments, the method further comprises administering to the subject a delivery-enhancing agent. In some embodiments, the delivery-enhancing agent is selected from the group consisting of a cyclodextrin, an inactivated bacterium, polyvinyl alcohol (PVA), an inulin or an ester thereof, and a combination thereof. In some embodiments, the inulin ester is selected from the group consisting of an inulin butyrate ester, an inulin propionate ester, and a combination thereof.

In some embodiments, the miR-22 inhibitor and the metabolism-enhancing agent are co-administered. In some embodiments, the miR-22 inhibitor and the metabolism-enhancing agent are administered sequentially.

In some embodiments, the metabolic disease is selected from the group consisting of alcoholic steatohepatitis (ASH), non-alcoholic steatohepatitis (NASH), non-alcoholic fatty liver disease (NAFLD), diabetes, obesity, dyslipidemia, and a combination thereof.

In some embodiments, a sample is obtained from the subject. In some embodiments, the sample comprises blood, tissue, or a combination thereof. In some embodiments, the tissue comprises diseased tissue. In some embodiments, the tissue comprises normal tissue.

In some embodiments, the level and/or activity of one or more biomarkers is measured in the sample. In some embodiments, the one or more biomarkers is selected from the group consisting of a miR, fibroblast growth factor 21 (FGF21), fibroblast growth factor receptor 1c (FGFR1c), sirtuin 1 (SIRT1), Beta-klotho, blood glucose, total cholesterol, low-density lipoprotein (LDL), high-density lipoprotein (HDL), triglyceride level, C-reactive protein, hemoglobin A1c, 5′-adenosine monophosphate-activated protein kinase (AMPK), peroxisome proliferator-activated receptor alpha (PPAR-α), peroxisome proliferator-activated receptor gamma coactivator 1-alpha (PGC-1a), aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, albumin, ferritin, collagen 1a1, cyclin A2, alpha-smooth muscle actin (αSMA), procollagen α1 (procol1), transforming growth factor-β (TGFβ), monocyte chemoattractant protein-1 (MCP1), interleukin-6 (IL-6), interleukin-10 (IL-10), interleukin-17 (IL-17), interleukin-1β (IL-1b), tumor necrosis factor alpha (TNFα), interferon-gamma (INF-γ), RAR-related orphan receptor gamma (ROR-γ), alpha-actin 1 (ACTA1), tissue growth factor beta (TGFβ), connective tissue growth factor (CTGF), platelet derived growth factor receptor beta (PDGFRβ), carnitine palmitoyltransferase 1 (CPT1), 3-hydroxy-3-methylglutaryl-CoA synthase 2 (mitochondrial) (HMGCS2), ERK1, ERK2, phosphoenolpyruvate carboxykinase (PEPCK), glucose-6-phosphatase (G6pase), a fatty acid binding protein (FAPB), fatty acid synthase (FASN), sterol regulatory element-binding protein 1 (SREBP1), cytochrome P450 4A11 (CYP4A11), glucagon-like peptide 1 (GLP1), peptide YY (PYY), zona occludens protein 1 (ZO-1), zona occludens protein 2 (ZO-2), zona occludens protein 3 (ZO-3), junctional adhesion molecule 1 (JAM-1), waist circumference, body mass index (BMI), homeostatic model assessment (HOMA) score, and a combination thereof. In some embodiments, the miR is miR-22.

In some embodiments, the measured level and/or activity of the one or more biomarkers in the sample is abnormal compared to a reference sample. In some embodiments, the reference sample is obtained from the subject. In some embodiments, the reference sample is obtained from a different subject or a population of subjects. In some embodiments, the level and/or activity of the one or more biomarkers is measured before and/or after the miR-22 inhibitor or the combination of the miR-22 inhibitor and the metabolism-enhancing agent is administered to the subject.

In some embodiments, the level and/or activity of FGF21 is increased in a liver tissue sample obtained from the subject after administration of the miR-22 inhibitor or combination of the miR-22 inhibitor and the metabolism-enhancing agent compared to a liver tissue sample obtained before administration of the miR-22 inhibitor or combination of the miR-22 inhibitor and the metabolism-enhancing agent.

In some embodiments, the level and/or activity of miR-22 is decreased in a sample obtained from the subject after administration of the miR-22 inhibitor or combination of the miR-22 inhibitor and the metabolism-enhancing agent compared to a sample obtained before administration of the miR-22 inhibitor or combination of the miR-22 inhibitor and the metabolism-enhancing agent. In some embodiments, the level and/or activity of miR-22 is decreased in a liver tissue sample obtained from the subject after administration of the miR-22 inhibitor or combination of the miR-22 inhibitor and the metabolism-enhancing agent compared to a liver tissue sample obtained before administration of the miR-22 inhibitor or combination of the miR-22 inhibitor and the metabolism-enhancing agent.

In some embodiments, the administration of the miR-22 inhibitor or the combination of the miR-22 inhibitor and the metabolism-enhancing agent to the subject improves one or more symptoms of the metabolic disease in the subject. In some embodiments, insulin sensitivity in the subject is improved.

In another aspect, the present invention provides a pharmaceutical composition. In some embodiments, the pharmaceutical composition comprises a microRNA-22 (miR-22) inhibitor, a metabolism-enhancing agent, and a pharmaceutically acceptable carrier. In some embodiments, the miR-22 inhibitor is an inhibitor of human miR-22 (hsa-miR-22).

In some embodiments, the miR-22 inhibitor is an oligonucleotide. In some embodiments, the oligonucleotide comprises a nucleic acid sequence that hybridizes to miR-22 and reduces miR-22 expression. In some embodiments, the oligonucleotide comprises unmodified and/or modified nucleotides. In some embodiments, the oligonucleotide comprises a nucleic acid sequence that has at least about 80% sequence identity to SEQ ID NO:1. In some embodiments, the oligonucleotide comprises the nucleic acid sequence set forth in SEQ ID NO:1.

In some embodiments, the metabolism-enhancing agent is selected from the group consisting of fibroblast growth factor 21 (FGF21), an FGF21 mimic, an FGF21 inducer, a retinoid, a histone deacetylase (HDAC) inhibitor, metformin, a bile acid or an analog thereof, a resistant starch, a prebiotic agent, a probiotic agent, and a combination thereof. In some embodiments, the retinoid is selected from the group consisting of retinoic acid (RA), retinol, retinal, isotretinoin, alltretinoin, etretinate, acitretin, tazarotene, bexarotene, adapalene, seletinoid G, a retinyl ester, fenretinide, derivatives thereof, and a combination thereof. In some embodiments, the retinyl ester is selected from the group consisting of retinyl acetate, retinyl butyrate, retinyl propionate, retinyl palmitate, and a combination thereof. In some embodiments, the retinoid is RA and the concentration of RA is about 10 μM.

In some embodiments, the HDAC inhibitor is selected from the group consisting of a short-chain fatty acid (SCFA), suberanilohydroxamic acid (SAHA), trichostatin, and a combination thereof. In some embodiments, the HDAC inhibitor is SAHA and the concentration of SAHA is about 5 μM. In some embodiments, the SCFA is selected from the group consisting of propionate, butyrate, isobutyrate, valerate, isovalerate, and a combination thereof. In some embodiments, the SCFA is butyrate. In some embodiments, the concentration of the SCFA is between about 5 mM and about 10 mM.

In some embodiments, the bile acid or analog thereof comprises obeticholic acid, a bile acid receptor FXR or TGR5 agonist. In some embodiments, the probiotic comprises a bacterium that produces an SCFA. In some embodiments, the prebiotic comprises apple pectin, an inulin, or a combination thereof. In some embodiments, the pharmaceutical composition comprises a therapeutically effective amount of the miR-22 inhibitor and/or the metabolism-enhancing agent.

In some embodiments, the pharmaceutical composition further comprises a delivery-enhancing agent. In some embodiments, the delivery-enhancing agent is selected from the group consisting of a cyclodextrin, an inactivated bacterium, polyvinyl alcohol (PVA), an inulin or an ester thereof, and a combination thereof. In some embodiments, the inulin ester is selected from the group consisting of an inulin butyrate ester, an inulin propionate ester, and a combination thereof. In some embodiments, the pharmaceutical composition comprises a nanoemulsion.

In some embodiments, the pharmaceutical composition is administered to a subject to prevent or treat a metabolic disease. In some embodiments, the metabolic disease is selected from the group consisting of alcoholic steatohepatitis (ASH), non-alcoholic steatohepatitis (NASH), non-alcoholic fatty liver disease (NAFLD), diabetes, obesity, dyslipidemia, and a combination thereof.

In another aspect, the present invention provides a kit for preventing of treating a metabolic disease in a subject. In some embodiments, the kit comprises a pharmaceutical composition disclosed herein. In some embodiments, the metabolic disease is selected from the group consisting of alcoholic steatohepatitis (ASH), non-alcoholic steatohepatitis (NASH), non-alcoholic fatty liver disease (NAFLD), diabetes, obesity, dyslipidemia, and a combination thereof. In some embodiments, the kit further comprises instructions for use.

Other objects, features, and advantages of the present invention will be apparent to one of skill in the art from the following detailed description and figures.

### BRIEF DESCRIPTION OF THE DRAWINGS

FIG. 1 shows that metabolic beneficial chemicals such as bile acids, retinoic acid, and butyrate can simultaneously induce both hepatic FGF21 and miR-22, which inhibits FGF21 and FGFR1 to maintain metabolism homeostasis. Western diet and alcohol super-induce hepatic miR-22, leading the development of metabolic disease.

FIGS. 2A-2C show the effects of the Western diet. FIG. 2A shows that Western diet (WD)-induced steatosis was much more severe in males than females, which was accompanied by the reduction of hepatic FGF21. Female mice, which have superior metabolism compared with males, always had higher hepatic FGF21 levels. FIG. 2B shows that Western diet-reduced hepatic FGF21 was associated with increased hepatic miR-22. FIG. 2C shows that ethanol induced hepatic miR-22 levels similar to the Western diet. n=6. * denotes p<0.05.

FIGS. 3A-3D show gender differences in metabolism. FIG. 3A shows FGF21 mRNA and protein expression. FIG. 3B shows miR-22 levels. FIG. 3C shows glucose levels and that female mice were more insulin sensitive than males. FIG. 3D shows expression of hepatic genes that are responsible for metabolism. n=6. Data is shown as mean+/−S.D. ** denotes p<0.05. *** denotes p<0.01.

FIGS. 4A-4C show FGF21 and miR-22 levels. FIG. 4A shows hepatic FGF21 mRNA and serum FGF21 levels in healthy individuals (BMI<25) and patients who had fatty livers (BMI>35). n=6 per group. FIG. 4B shows hepatic and serum miR-22 levels in lean (BMI<25) and obese men who had fatty livers (BMI>35). FIG. 4C shows hepatic FGF21 mRNA and protein levels and serum FGF21 in mice fed with control diet (healthy) and Western diet (fatty liver). In both cases there was an inverse relationship between the levels in the liver and serum. n=about 4 to 7 per group. * denotes p<0.05. The serum and hepatic FGF21 and miR-22 levels were inversely correlated.

FIGS. 5A-5C show the effect of bile acids in inducing miR-22 levels. FIG. 5A shows a dose response experiment in which 50, 100, and 150 μM of CDCA were used to treat cells for 24 hours. A time course study in which HCT116 cells were treated by 150 μM CDCA for indicated times. FIG. 5B shows the levels of FGF21, a key metabolic regulator, and miR-22 in CDCA (150 μM)-treated Huh7 and HCT116 cells within 24 hours. FIG. 5C shows that obeticholic acid (OCA, 25 and 50 μM) induced miR-22 in HCT116 cells after 24 hours of treatment. Data are presented as mean+/−S.D. * denotes p<0.05.

FIGS. 6A-6C show the effects of retinoic acid (RA) and butyrate. FIG. 6A shows that RA and butyrate improved insulin sensitivity. FIG. 6B shows that RA and butyrate increased miR-22. FIG. 6C shows that RA and butyrate induced hepatic PGC1α mRNA in C57BL/6 male mice. n=5. * denotes p<0.01.

FIGS. 7A and 7B show the effects of retinoic acid (RA) (1 μM) and butyrate (3 mM) in Huh7 cells. FIG. 7A shows that RA and butyrate together induced miR-22, FGF21, FGFR1, and PGC1α mRNA levels. FIG. 7B shows the effect of RA and butyrate in regulating the levels of indicated proteins in human liver Huh7 cells. Data is shown as mean+/=S.D. * denotes p<0.05.

FIG. 8 shows that miR-22 mimics reduced the protein levels of FGF21 and FGFR1, the receptor of FGF21. The net effect of miR-22 mimics was a reduction in P-AMPK, thereby reducing metabolism in Huh7 cells.

FIGS. 9A and 9B show the effects of miR-22 inhibitors on FGF21, FGFR1, and P-AMPK. FIG. 9A shows that miR-22 inhibitors further enhanced the effects of RA and butyrate in inducing FGF21, FGFR1, and P-AMPK. FIG. 9B shows that increased FGF21 and FGFR1 was only noted in miR-22 inhibitor-infected cells, which had positive GFP labeling.

FIG. 10 shows that histological data revealed that miR-22 mimics increased the level of steatosis and induced hepatic lymphocyte infiltration, whereas miR-22 inhibitors reduced hepatic fat in Western diet-fed mice. In addition, hepatic PGC1α level was reduced by miR-22 mimics, but increased by miR-22 inhibitors. Data is shown as mean+/−S.D. * denotes p<0.05.

FIGS. 11A and 11B show the effects of miR-22 inhibitors in alcohol-induced steatosis. FIG. 11A shows that liver histology revealed that miR-22 inhibitors reversed alcohol-induced steatosis in C57BL/6 mice. FIG. 11B shows the hepatic miR-22 levels in each group quantified by qPCR. Data is shown as mean+/−S.D. * denotes p<0.05.

FIG. 12 depicts metabolic homeostasis regulated by AMPK activators INT-747, as well as FGF21 and AMPK silencer miR-22.

FIG. 13 shows the results of insulin tolerance testing done in 3-month-old male wild-type and hepatocyte FGF21 KO mice. Fed or 6-hour-fasted mice received insulin (1 U/kg body weight). Blood glucose level was measured at 0, 30, 60, 90, and 120 minutes after insulin injection. The area under the curve (AUC) was calculated. Data is shown as mean+/−SE. n=4. * denotes p<0.01.

FIGS. 14A-14C show the effect of FGF21 on liver metabolism and regeneration. FIG. 14A shows that compared with control diet (CD)-fed mice, Western diet (WD)-fed mice had reduced proteins 24 hours after partial hepatectomy. FIG. 14B shows that forced FGF21 expression by tail vein injection after adenoviral FGF21 eliminated fat and rescued delayed regeneration in WD-fed mice; whereas knockdown of FGF21 by adenoviral siRNA (siFGF21) exacerbated steatosis and reduced the number of Ki-67-positive cells. The numbers are averages of Ki-67-positive cells per field. FIG. 14C shows that increased hepatic FGF21 lead to ERK1/2 activation. n=5.

FIGS. 15A-15C show the effects of the Western diet. FIGS. 15A and 15B show that Western diet (WD)-induced fatty livers had reduced FGF21 and SIRT1, but increased miR-22 levels in the livers and adipose tissues in mice (n=6). FIG. 15C shows that miR-22 levels in human livers with different percentage of fat evaluated by a pathologist (n=7 per group). * denotes p<0.05.

FIG. 16 shows the effect of miR-22 on indicated protein levels in Huh7 cells.

FIGS. 17A and 17B show that tail vein injection of adenoviral-delivered miR-22 inhibitors reversed Western diet (WD)- and alcohol-induced steatosis in C57BL/6 mice. FIG. 17A shows the livers of WD-fed mice that received adenoviral control, miR-22, or miR-22 inhibitor via tail vein. miR-22 induced steatosis and induced lymphocyte infiltration, whereas miR-22 inhibitor-treated mice had normal livers. FIG. 17B shows that fatty livers were induced by feeding 3-month-old mice with Liber DeCarli liquid diet supplemented with 5% alcohol for 3 weeks, but reversed by miR-22 inhibitor treatment via tail vein injection (n=5).

FIG. 18 shows that INT-747 simultaneously induced FGF21 signaling and miR-22 in Huh7 cells. Shown are the mRNA levels of miR-22, FGF21, and FGFR1 along with indicated protein levels in Huh7 cells treated with and without INT-747 for 6 hours. * denotes p<0.05. ** denotes p<0.01. *** denotes p<0.001.

FIG. 19 shows that butyrate simultaneously induced FGF21, FGFR1, βKlotho, and miR-22 in Huh7 cells. Shown are the indicated mRNA and protein levels in Huh7 cells treated with or without butyrate (3 mM) for 24 hours. * denotes p<0.05.

FIGS. 20A and 20B show the effects of butyrate. FIG. 20A shows that butyrate reduced the expression of hepatic pro-inflammatory genes and increased the expression of metabolic genes. FIG. 20B shows that butyrate increased FXR and FGF15 protein. Healthy C57BL/6 male mice were supplemented with or without butyrate (1 g/kg body weight) orally for 7 days. n=4. Data shown as mean+/−S.D. * denotes p<0.05. *** denotes p<0.001.

FIG. 21 shows that HDAC inhibitors including SAHA (5 butyrate (5 mM), propionate (10 mM), and valerate (10 mM) induced miR-22 in HCT116 cells after 1-day treatment. Data shown as mean+/−S.D. * denotes p<0.05. ** denotes p<0.01.

FIG. 22 shows the effect of miR-22 on the indicated protein level in human liver Huh7 and human colon HCT116 cells. Cyclin A2 is a known miR-22 target that was included as a control.

FIGS. 23A-23D show gender differences in metabolism. FIG. 23A shows that compared with male mice, female mice were more sensitive to insulin. FIGS. 23B-23D show that female mice also had higher expression levels of hepatic FGF21, miR-22, and metabolism genes.

FIGS. 24A and 24B show that OCA induced FGF21 signaling by increasing the expression of FGF21, FGF21 receptor (FGFR1) and PGC1α, which acts upstream of FGF21. FIG. 24A shows mRNA levels. FIG. 24B shows protein levels. * indicates p<0.05, ** p<0.01, *** p<0.0001 in treatment vs. DMSO control.

FIGS. 25A and 25B show that retinoic acid (RA) plus butyrate induced FGF21 signaling by increasing the expression of FGF21, FGF21 receptor (FGFR1) and PGC1α, which acts upstream of FGF21. FIG. 25A shows mRNA levels. FIG. 25B shows protein levels. * indicates p<0.05, ** p<0.01, *** p<0.0001 in treatment vs. DMSO control; # indicated p<0.05 in combination treatment vs. single treatment.

FIG. 26 shows inverse expression patterns between FGF21 signaling and miR-22 in human fatty liver.

FIGS. 27A and 27B show that FGFR1 was identified as a miR-22 direct target and that miR-22 reduced both FGFR1 and FGF21. FIG. 27A shows the results of luciferase activity assays. Data are presented as mean±SD with ** indicating p<0.01. FIG. 27B shows mRNA and protein expression levels. The hsa-miR-22 sequence is set forth in SEQ ID NO:2 and the FGFR1 3′ UTR sequence is set forth in SEQ ID NO:3.

FIGS. 28A and 28B show that miR-22 inhibition further enhances the effect of OCA and RA plus butyrate in inducing FGF21 signaling. FIG. 28A shows protein expression of indicated genes. FIG. 28B shows immunostaining, using a fluorescent-tagged secondary antibody.

FIGS. 29A-29E show that adenovirus delivery of a miR-22 inhibitor rescued alcohol-induced steatosis in C57BL/6 mice. FIG. 29A shows representative H&E-stained liver sections. FIG. 29B shows steatosis scores and ballooning scores. FIG. 29C shows hepatic cholesterol levels. FIG. 29D shows hepatic triglyceride levels. FIG. 29E shows mRNA expression of various genes. Data are shown as mean±SD. One-way ANOVA with Tukey's correction. * p<0.05, ** p<0.01, *** p<0.0001 (n=8 to 12 for each group).

FIG. 30 shows the combination effect of retinoic acid and short chain fatty acids, which have histone deacetylase (HDAC) inhibitory properties, in inducing miR-22 in HCT116 cells. SAHA was included as a positive control. Data are shown as mean+/−S.D. * p<0.05; ** p<0.01; ***p<0.001.

FIGS. 31A-31C show the effect of bile acid chenodeoxycholic acid (CDCA) in inducing miR-22 levels in HCT116 cells. FIG. 31A shows a dose response experiment in which 50, 100, or 150 μM of CDCA were used to treat cells for 24 hours. FIG. 31B shows a time course study in which HCT116 cells were treated with 150 μM CDCA for the indicated times. FIG. 31C shows the levels of FGF21, a key metabolic regulator, and miR-22 in CDCA (150 μM) treated Huh7 and HCT116 cells. Data are shown as mean+/−S.D. * p<0.05; ** p<0.01; ***p<0.001.

FIGS. 32A-32C show the effects of obeticholic acid (OCA), a potent bile acid receptor FXR agonist also known as INT-747. FIG. 32A shows that OCA induced miR-22 and induced FXR target FGF19, as expected, in HCT116 cells. It also reduced CCNA2, which is a validated miR-22 target. OCA is a semi-synthetic bile acid, which as the structure of 6α-ethyl-chenodeoxycholic acid. FIG. 32B shows the mRNA levels of indicated genes at indicated times in OCA-treated Huh7 cells (5 and 20 FIG. 32C shows the levels of indicated proteins after treatment of Huh7 cells with OCA for 6 hours. Data are shown as mean+/−S.D. * p<0.05; ** p<0.01; *** p<0.001. For FIGS. 32B and 32C, comparison is to DMSO control. # indicates p<0.05 in combination treatment vs. single treatment.

FIGS. 33A-33C show the effects of RA and butyrate. FIG. 33A shows that retinoic acid (RA) (1 μM) and butyrate (3 mM) together induced FGF21 signaling by increasing the expression of FGF21, FGF21 receptor (FGFR1), FGF21 binding protein βKlotho, and PGC1α, an upstream target of FGF21 and FGFR1. The net effect was to activate AMPK and stimulate growth, as evidenced by increased P-AMPK and p-ERK1/2 in Huh 7 cells shown in Western blots. Huh7 cells were treated with and without RA/butyrate for 6 hours. FIG. 33B shows that miR-22 inhibitors further enhanced the effects of RA and butyrate in inducing FGF21, FGFR1, P-AMPK, and p-ERK1/2. FIG. 33C shows that increased FGF21 and FGFR1 was only observed in miR-22 inhibitor-infected cells, which had positive GFP labeling. Data shown as mean+/−S.D. These findings show that the induction of miR-22 terminated the effect of RA and butyrate avoiding metabolism-driven overgrowth. These findings also show that a miR-22 inhibitor may increase the effects of OCA.

FIG. 34 shows miR-22 expression in wild-type and FGF21 KO mice (24 months old vs. 6 months old) N=4. * p<0.05.

FIG. 35 shows miR-22 expression in C57BL/6 male wild-type mice and hepatic FGF21 KO mice that were fed with Lieber-DeCarli liquid diet for 5 days, then mice randomly divided into pair-fed and ethanol-fed (5% ethanol) groups.

FIG. 36 shows that miR-22 levels were increased in human fatty livers. In addition, the levels of hepatic miR-22 were inversely correlated with the levels of PGC1α, a master metabolic regulator. The amount of fat in human livers was determined by histological evaluation. N=4 or 5 per group. * p<0.05. These findings show that overexpression of miR-22 may contribute to the development of fatty liver.

FIGS. 37A-37D show the effect of the Western diet (WD). FIG. 37A shows that WD-induced fatty livers had reduced SIRT1, FGF21, activated ERK1/2, and reduced cyclin D, accompanied by reduced liver regeneration capability one day after 2/3 liver resection. FIG. 37B shows that forced FGF21 expression by tail vein injection of Ad-FGF21 eliminated fat and rescued delayed regeneration in WD-fed mice, whereas FGF21 knockdown by siFGF21 exacerbated WD-induced steatosis accompanied by reduced Ki67-positive cells. The numbers are averages of Ki67-positive cells per field of vision. FIG. 37C shows that forced FGF21 expression also activated p-ERK1/2, therefore increasing metabolism-driven growth. FIG. 37D shows that hepatic miR-22 levels were positively associated with the severity of steatosis. N=5. * p<0.05. These findings indicate that miR-22 inhibitors can be used to improve metabolism of fatty livers in order to stimulate liver regeneration.

FIG. 38 shows expression of miR-22 and various genes. Mice received miR-22 inhibitors or miR-22 mimics via tail vein injection 3 days prior to 2/3 liver resection (1×10⁹ pfu in 100 μL saline, one injection per day). Mice were killed on day 0 (immediately after liver resection) or 2 days later.

FIG. 39 shows the effects of miR-22 inhibitors on liver and body weight. Mice were treated as described in FIG. 38. The liver-to-body weight ratio as well as the fat-to-body weight ratio were substantially higher in miR-22 mimic-treated mice compared to other groups. N=4. * p<0.05.

FIGS. 40A-40D show elevated miR-22 is accompanied by reduced CCNA2, FGF21, FGFR1, and PGC1α in human and mouse steatosis livers. FIG. 40A: Hepatic miR-22, CCNA2, FGF21, FGFR1, and PGC1α mRNA levels as well as serum FGF21 concentration in healthy people and patients who had fatty liver. Steatosis was graded by a pathologist based on fat content: grade 0 (normal)≤5%; grade 1 (mild)=5%-33%; grade 2 (moderate)=34%-66%; grade 3 (severe)≥67%, n=8-9 livers per group; FIG. 40B: Relationships between the expression levels of indicated genes and hepatic fat content. FIG. 40C: Liver histology revealed that a Western diet (WD) induced steatosis in C57BL/6 mice. FIG. 40D: Hepatic miR-22, Ccna2, Fgf21, Fgfr1, and Pgc1α mRNA levels as well as serum FGF21 concentration in control diet (CD) or WD-fed mice. Data=mean±SD, n=8 mice per group. * p<0.05, ** p<0.01, ***p<0.0001.

FIGS. 41A-41E show the mechanisms by which miR-22 reduces FGFR1 and FGF21. FIG. 41A: The level of indicated proteins and mRNAs in Huh7 cells 48 h post adenoviral-miR-22 (miR-22) or negative adenoviral (control) infection. FIG. 41B: miR-22, which is conserved between humans and mice, partially pairs with the 3′UTR of the FGFR1. Adenovirus negative control (control), adenoviral miR-22 (miR-22), or adenoviral miR-22 inhibitors (miR-22 inhibitors) were used to infect Huh7 cells for 48 h followed by transfection of reporter constructs containing the 3′UTR of the FGF21 or FGFR1 cloned into psiCHECK2. psiCHECK2 without insert was used as a negative control. Reporter assay revealed that miR-22 reduced the luciferase activity driven by FGFR1 3′UTR, but not by FGF21 3′UTR. Thus, miR-22 directly silences FGFR1 expression. FIG. 41C: PPARα and PGC1α protein levels in Huh7 cells at 48 h post infection of control or miR-22. FIG. 41D: There are two peroxisome proliferative-response elements (PPREs) present in the regulatory region of both human and mouse FGF21 gene. FIG. 41E: Huh7 cells were infected with miR-22 or control followed by chromatin isolation. ChIP was performed on cell lysates using indicated antibodies followed by qPCR using FGF21-specific primers. Binding was expressed relative to the IgG antibody that was used as a negative control. ChIP-qPCR data revealed that miR-22 reduced the recruitment of PPARα and PGC1α to the PPREs. Data are presented as the mean±SD with ** p<0.01; ***p<0.001.

FIGS. 42A-42E show miR-22 inhibitors treat alcoholic steatosis by inducing FGF21-mediated AMPK activation. Three-month-old C57BL/6 male mice were fed with Liber DeCarli diet supplemented with and without 5% alcohol for 3 weeks. The alcohol-fed mice were treated with miR-22 inhibitors (1×10⁹ PFU, via tail vein, 3 times in 10 days) or adenovirus serving as a negative control. All the mice were euthanized 1 day after the last viral injection. FIG. 42A: Representative H&E-stained liver sections; FIG. 42B: Steatosis scores; FIG. 42C: Hepatic cholesterol level; FIG. 42D: Hepatic triglyceride level; FIG. 42E: Hepatic miR-22 as well as indicated mRNA and protein levels in each group. Hepatic fat content was scored as 0 (<5%), 1 (5-33%), 2 (34-66%), and 3 (>67%). Data are shown as mean±SD. * p<0.05, ** p<0.01, ***p<0.0001 (n=8-12 for each group).

FIGS. 43A-43B show miR-22 inhibitors do not have proliferative effect and apparent toxicity in mice. Three-month-old male and female C57BL/6 mice received adenovirus control or miR-22 inhibitors (1×10⁹ PFU, tail vein injection, once/week) for 4 months. Age- and sex-matched mice without any treatment were used as baseline controls. After the last viral injection, mice were injected with BrdU (i.p.) and euthanized 2 h later. FIG. 43A: Body weight gain, liver-to-body weight ratio, blood glucose level after 6 h fasting; serum ALT, ALP, and endotoxin (LPS) levels; FIG. 43B: Representative liver sections of BrdU staining; BrdU-positive cells were counted in five random fields (>200 cells/field) per liver section (3 mice per group). Data are expressed as mean±SD. One-way ANOVA with Tukey's correction.

FIGS. 44A-44C show obeticholic acid (OCA) simultaneously induces miR-22, a metabolic silencer, as well as metabolic facilitators FGF21, FGFR1, and PGC1α in Huh7 cells. Inhibiting miR-22 enhances OCA-induced FGF21 signaling leading to AMPK and ERK1/2 activation. miR-22 as well as indicated mRNA (FIG. 44A) and protein (FIG. 44B) levels in Huh7 cells treated with DMSO or OCA for 6 h. *p<0.05, ***p<0.0001 OCA vs. DMSO. FIG. 44C: Protein levels in Huh7 cells infected with adenovirus negative control or miR-22 inhibitors followed by DMSO or OCA (5 μM) treatment for 6 h.

FIGS. 45A-45G show miR-22 inhibitors enhance the effect of OCA in reducing steatosis, improving insulin sensitivity, and inducing FGF21 signaling. C57BL/6 male mice were fed with a WD since weaning. When those mice were 7-months old, they received OCA (10 mg/g body weight, daily oral gavage), adenovirus negative control, or miR-22 inhibitors (1×10⁹ PFU, tail vein injection, once a week), or a combination of OCA plus miR-22 inhibitors for 3 weeks. Age- and sex-matched CD-fed mice without any treatment were used as baseline controls. FIG. 45A: Representative gross liver morphology and H&E-stained liver sections; FIG. 45B: Steatosis scores; FIG. 45C: Glycemic response measured by insulin tolerance test (ITT); FIG. 45D: Serum GLP-1 secretion; FIG. 45E: Serum FGF21 level; FIG. 45F: Hepatic cholesterol and triglyceride levels; FIG. 45G: Serum ALT, ALP, and endotoxin (LPS) levels. Hepatic fat content was scored as 0 (<5%), 1 (5-33%), 2 (34-66%), and 3 (>67%). Data are shown as mean±SD (n=4). One-way ANOVA with Tukey's correction. ^{#}p<0.05, ^{##}p<0.01, ^{###}p<0.0001 between WD and CD; *p<0.05, **p<0.01, ***p<0.0001 between treated groups and controls; ^{$}p<0.05, ^{$$} p<0.01, ^{$$$} p<0.0001 between combination and single treatment.

FIGS. 46A-46B show the effects of miR-22 inhibitors and OCA in regulating the expression of hepatic genes and proteins implicated in metabolism as well as fatty acid synthesis and uptake. Mice were treated using the same method described above. Hepatic levels of indicated proteins (FIG. 46A) and mRNAs (FIG. 46B) in the CD-fed and WD-fed mice. Data are shown as mean±SD (n=4). One-way ANOVA with Tukey's correction. ^{#}p<0.05, ^{##}p<0.01, ^{###}p<0.0001 between WD and CD; *p<0.05, **p<0.01, ***p<0.0001 between treated and their controls; ^{$}p<0.05, ^{$$} p<0.01, ^{$$$} p<0.0001 between combination and single treatment.

FIG. 47 shows metabolic disease development and treatment controlled by miR-22 silenced and FXR activated FGF21 and FGFR1. Left panel: Overexpressed miR-22 likely contributes to the development of steatosis, but it also has a tumor suppressive effect by limiting ERK1/2 activation. Right panel: Activation of FXR facilitates metabolism and treats steatosis as well as improves inulin sensitivity, but it also induces miR-22, a metabolic inhibitor that silences FGF21 and FGFR1. The induction of miR-22 restricts FGF21-driven growth controlled by ERK1/2 activation. It also maintains FGF21 homeostasis. miR-22 inhibitors can be used to increase FGF21 and FGFR1 signaling in a metabolic compromised condition.

### DETAILED DESCRIPTION OF THE INVENTION

### I. Introduction

The present invention is based, in part, on the discovery that microRNA-22 (miR-22) is induced in fatty liver, and furthermore, that miR-22 inhibits the metabolism master regulator fibroblast growth factor 21 (FGF21). Thus, miR-22 inhibitors can be used to prevent and treat metabolic diseases including, but not limited to, steatosis, steatohepatitis, diabetes, obesity, metabolic syndrome, dyslipidemia, and conditions associated with fatty liver disease (e.g., non-alcoholic fatty liver disease (NAFLD) and non-alcoholic steatohepatitis (NASH)), as well as improve outcomes following liver transplantation. The present invention is also based, in part, on the discovery that miR-22 inhibitors can be used in conjunction with other agents that produce beneficial metabolic health effects, such as bile acids, metformin, retinoids, and histone deacetylase inhibitors such as short chain fatty acids.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to teach and provide written description support for a claim limitation of, e.g., “0.98X.”

The term “metabolic disease” refers to any disease or disorder that disrupts normal metabolism, including any disease that disrupts or dysregulates biochemical reactions that function to convert food into energy, process or transport amino acids, proteins, carbohydrates (e.g., sugars, starches), or lipids (e.g., fatty acids), etc. In some embodiments, a metabolic disease results in the abnormal processing or regulation of sugars, lipids, cholesterol, and/or the metabolism of drugs (e.g., by the liver). Non-limiting examples of metabolic diseases include obesity, insulin resistance, diabetes (e.g., type 2 diabetes), pre-diabetic conditions, dyslipidemia (e.g., hyperlipidemia), fatty liver diseases (e.g., non-alcoholic fatty liver disease (NAFLD), non-alcoholic steatohepatitis (NASH), and alcohol-induced fatty liver diseases), steatosis (e.g., non-alcoholic steatosis and alcohol-induced steatosis), and metabolic syndrome, as well as the sequelae of such diseases. Alcohol-related metabolic and hepatic diseases are also included.

The term “microRNA” or “miR” refers to a small non-coding RNA molecule (e.g., containing about 22 nucleotides) found in plants, animals, and some viruses, that functions in RNA silencing and post-transcriptional regulation of gene expression. A non-limiting example is miR-22 (e.g., SEQ ID NO:2).

The “miR-22” family, belonging to gene family number MIPF0000053, contains about 50 sequences across various species (see, e.g., http://www.mirbase.org/cgi-bin/mirna_summary.pl?fam=MIPF0000053). The coding sequence for human miR-22 (hsa-miR-22) is located on chromosome 17. Non-limiting examples of human miR-22 sequences include hsa-miR-22-3p (miRBASE accession number MIMAT0000077; SEQ ID NO:2), hsa-miR-22-5p (miRBASE accession number MIMAT0004495), and the hsa-miR-22 stem loop sequence (miRBASE accession number MI0000078).

The term “miR-22 inhibitor” refers to any agent that inhibits or decreases the expression, stability, or activity of miR-22. In some embodiments, a miR-22 inhibitor decreases or abolishes the expression (e.g., transcription) of miR-22. In some embodiments, a miR-22 inhibitor decreases the stability of a miR-22 RNA molecule or promotes the degradation of a miR-22 RNA molecule. In some embodiments, a miR-22 inhibitor decreases or prevents the binding of a miR-22 RNA molecule (e.g., to a binding target). In some embodiments, a miR-22 inhibitor is an oligonucleotide (e.g., an antisense oligonucleotide), which can, as a non-limiting example, comprise the nucleic acid sequence set forth in SEQ ID NO:1. In some embodiments, a miR-22 inhibitor is a small molecule compound that binds to miR-22 and decreases or abolishes its activity.

The term “metabolism-enhancing agent” refers to a compound or composition that promotes or maintains normal metabolism, or ameliorates the causes or sequelae of a metabolic disease. In some embodiments, a metabolism-enhancing agent prevents or treats, either alone or in combination with one or more additional agents, a metabolic disease. In some embodiments, a metabolism-enhancing agent is a compound or composition that increases or promotes FGF21 and/or FGFR1 expression or activity (e.g., FGF21 signaling). In some embodiments, a metabolism-enhancing agent is a compound or composition that functions as a histone deacetylase inhibitor. In some embodiments, a metabolism-enhancing agent is a compound or composition (e.g., metformin) that increases or promotes 5′ adenosine monophosphate-activated protein kinase (AMPK) expression or activity (e.g., AMPK signaling). In some embodiments, a metabolism-enhancing agent is a compound or composition that increases or promotes sirtuin 1 (SIRT1) expression or activity (e.g., SIRT1 signaling). In some embodiments, a metabolism-enhancing agent is a compound or composition that increases or promotes Beta-klotho expression or activity (e.g., Beta-klotho signaling). In some embodiments, a metabolism-enhancing agent is a compound or composition that increases or promotes bile acid receptor (FXR) expression or activity (e.g., FXR signaling).

The term “retinoid” refers to a class of compounds that are vitamers of vitamin A (i.e., compounds that generally have a similar structure to vitamin A) or are chemically related to vitamin A. Retinoids include, but are not limited to, any natural or synthetic derivative of retinol.

The term “histone deacetylase” or “HDAC” refers to a class of enzymes (Enzyme Commission number 3.5.1.98) that remove acetyl groups from proteins, including ε-N-acetyl lysine amino acids on histones. Histone deacetylation allows histones to wrap and compact DNA more tightly within chromatin, which is associated with gene silencing. Class I HDACs include HDAC1, HDAC2, HDAC3, and HDAC8. Class IIA HDACs include HDAC4, HDAC5, HDAC7, and HDAC9. Class IIB HDACs include HDAC6 and HDAC10. Class III HDACs include SIRT1, SIRT2, SIRT3, SIRT4, SIRT5, SIRT6, and SIRT7 in mammals and Sir2 in yeast. Class IV HDACs include HDAC11.

In humans, HDAC1 is encoded by the HDAC1 gene. A non-limiting example of a human HDAC1 amino acid sequence is set forth under GenBank Accession number NM_004964.2→NP_004955.2. In humans, HDAC4 is encoded by the HDAC4 gene. A non-limiting example of a human HDAC4 amino acid sequence is set forth under GenBank Accession number NM_006037.3→NP_006028.2. In humans SIRT1 is encoded by the SIRT1 gene. Non-limiting examples of human SIRT1 amino acid sequences are set forth under GenBank Accession number NM_001142498.1→NP_001135970.1, NM_001314049.1→NP_001300978.1, and NM_012238.4→NP_036370.2.

The term “histone deacetylase inhibitor” of “HDAC inhibitor” refers to any natural or synthetic compound or agent that decreases or suppresses the activity and/or expression of an HDAC. In some embodiments, an HDAC inhibitor decreases or suppresses the mRNA expression of an HDAC (e.g., transcription from a gene encoding an HDAC is decreased or suppressed). In some embodiments, an HDAC inhibitor decreases or suppresses the protein expression of an HDAC (e.g., translation of an mRNA expressed from an HDAC gene is decreased or suppressed). In some embodiments, an HDAC inhibitor decreases or suppresses the enzymatic activity of an HDAC. In some embodiments, an HDAC inhibitor decreases or suppresses the ability of an HDAC to deacetylate a protein, e.g., a histone.

Non-limiting examples of HDAC inhibitors include suberanilohydroxamic acid (SAHA), short-chain fatty acids (e.g., propionate, butyrate, isobutyrate, valerate, isovalerate), entinostat, panobinostat, trichostatin A, Scriptaid, mocetinostat, chidamide, TMP195, citarinostat, belinostat, depsipeptide, MC1568, tubastatin, givinostat, dacinostat, CUDC-101, JNJ-26481585, pracinostat, PCI-34051, PCI-34051, droxinostat, abexinostat, RGFP966, AR-42, ricolinostat, valproic acid, tacedinaline, CUDC-907, curcumin, M344, tubacin, RG2833, resminostat, divalproex, sodium phenylbutyrate, TMP269, CAY10683, tasquinimod, BRD73954, splitomicin, HPOB, LMK-235, nexturastat A, (−)-parthenolide, CAY10603, 4SC-202, BG45, and ITSA-1.

The terms “subject,” “individual,” and “patient” are used interchangeably herein to refer to a vertebrate, preferably a mammal, more preferably a human. Mammals include, but are not limited to, murines, rats, simians, humans, farm animals, sport animals, and pets. Tissues, cells and their progeny of a biological entity obtained in vivo or cultured in vitro are also encompassed.

The term “nanoemulsion” refers to a colloidal particulate system in the submicron size range. Nanoemulsions are particularly useful for acting as carriers in drug molecule delivery. Sizes range from about 10 nm to about 1,000 nm (e.g., about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1,000 nm).

The term “delivery-enhancing agent” refers to any compound or composition that promotes delivery, stability, availability, or effectiveness of an active agent (e.g., a miR-22 inhibitor and/or a metabolism-enhancing agent). In some embodiments, a delivery-enhancing agent increases the ability of an active agent to reach a target cell or tissue. In some embodiments, a delivery-enhancing agent increases the stability of an active agent or protects an active agent from degradation or metabolism. As a non-limiting example, a delivery-enhancing agent may protect an active agent from digestion in the gut until the active agent reaches the desired target cell or tissue. In some embodiments, a delivery-enhancing agent reduces the amount of an active agent that is needed in order to achieve the desired effect (e.g., therapeutic effect). In some embodiments, a delivery-enhancing agent increases the solubility of an active agent. In some embodiments, a delivery-enhancing agent increases the bioavailability of an active agent or increases the retention time of an active agent (e.g., within a subject following administration).

As used herein, the term “administering” includes oral administration, topical contact, administration as a suppository, intravenous, intraperitoneal, intramuscular, intralesional, intrathecal, intranasal, intraosseous, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intraosseous, and intracranial. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “therapeutically effective amount” or “sufficient amount” refers to the amount of a miR-22 inhibitor, metabolism-enhancing agent, or composition that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the target cell type, the location of the target cell in the subject, the dosing regimen to be followed, whether it is administered in combination with other compounds, timing of administration, and the physical delivery system in which it is carried.

For the purposes herein an effective amount is determined by such considerations as may be known in the art. The amount must be effective to achieve the desired therapeutic effect in a subject suffering from a metabolic disease. The desired therapeutic effect may include, for example, improvement in or amelioration of undesired symptoms associated with the metabolic disease, prevention of the manifestation of such symptoms before they occur, slowing down the progression of symptoms associated with the metabolic disease, slowing down or limiting any irreversible damage caused by the metabolic disease, lessening the severity of or curing the metabolic disease, or improving the survival rate or providing more rapid recovery from the metabolic disease. Further, in the context of prophylactic treatment the amount may also be effective to prevent the development of the metabolic disease.

The term “pharmaceutically acceptable carrier” refers to a substance that aids the administration of an active agent (e.g., a miR-22 inhibitor and/or a metabolism-enhancing agent) to a cell, an organism, or a subject. “Pharmaceutically acceptable carrier” refers to a carrier or excipient that can be included in the compositions of the invention and that causes no significant adverse toxicological effect on the patient. Non-limiting examples of pharmaceutically acceptable carrier include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor, etc. In some instances, the carrier is an agent that facilitates the delivery of a miR-22 inhibitor, metabolism-enhancing agent, or composition to a target cell or tissue. One of skill in the art will recognize that other pharmaceutical carriers are useful in the present invention.

The term “nucleic acid” as used herein refers to a polymer containing at least two deoxyribonucleotides or ribonucleotides in either single- or double-stranded form and includes DNA, RNA, and hybrids thereof. DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. RNA may be in the form of small interfering RNA (siRNA), Dicer-substrate dsRNA, small hairpin RNA (shRNA), asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)). “Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “prodrug” means a medication or compound that, after administration, is converted to a biologically or pharmacologically active form. Conversion of the prodrug to the active compound is typically the result of metabolism within the body. As a non-limiting example, tributyrin is a prodrug of butyric acid, of which butyrate is the conjugate base.

The term “alpha-smooth muscle actin or “αSMA” refers to a protein that is encoded by the ACTA2 gene, and is commonly used as a marker of myofibroblast formation. αSMA belongs to the actin family of proteins, which are highly conserved and play roles in cell motility, structure, and integrity. The gene encoding αSMA is also known as AAT6, ACTSA, or MYMY5, and is located on human chromosome 10. Non-limiting examples of αSMA amino acid sequences are set forth in GenBank Accession Nos. NM_001141945.2→NP_001135417.1 (human), NM_001613.2→NP_001604.1 (human), and NM_007392.3→NP_031418.1 (mouse).

The term “monocyte chemoattractant protein-1” or “MCP1” refers to a small cytokine that belongs to the CC chemokine family and is encoded by the CCL2 gene. MCP1 is also known as chemokine (C—C motif) ligand 2 (CCL2) or small inducible cytokine A2. The CCL2 gene is found on chromosome 17 in humans. MCP1 recruits monocytes, memory T cells, and dendritic cells to sites of inflammation. Non-limiting examples of MCP1 amino acid sequences are set forth in GenBank Accession Nos. NM_002982.3→NP_002973.1 (human) and NM_011331.2→NP_035461.2 (mouse).

The term “procollagen α1” or “procol1” refers to a protein encoded by the COL1A1 gene. A chain of procollagen α1 combines with a second chain of procollagen α1 and one chain of procollagen α2 (encoded by the COL1A2 gene) to form type I procollagen, which is then processed into type I collagen. Type I collagen is a fibrillary collagen found in most connective tissues in the body, including cartilage. Procol1 expression is associated with the development of fibrosis. Non-limiting examples of procol1 amino acid sequences are set forth in GenBank Accession Nos. NM_000088.3→NP_000079.2 (human) and NM_007742.4→NP_031768.2 (mouse).

The term “interleukin-10” or “IL-1b” refers to a cytokine that is also known as leukocytic pyrogen, mononuclear cell factor, lymphocyte activating factor, IL1B, IL-1, IL1-BETA, or IL1F2, and is encoded by the IL1B gene. IL-1b plays roles in the inflammatory response and is involved in cell proliferation, differentiation, and apoptosis, as well as the development of fibrosis.

The term “transforming growth factor-β” or “TGFβ” refers to a multifunctional cytokine of the transforming growth factor superfamily that includes three different isoforms (TGFβ-1, TGFβ-2, and TGFβ-3. The three isoforms are encoded by the genes TGFB1, TGFB2, and TGFB3, respectively. TGFβ plays roles in cell proliferation, wound healing, and synthesis of extracellular matrix molecules. TGFβ is associated with the development of fibrosis in many different organs via its promotion of mesenchymal cell proliferation, migration, and accumulation following an inflammatory response.

The term “tumor necrosis factor alpha” or “TNFα” refers to the cytokine encoded by the gene TNF (also known as TNFA). TNFα is also known as tumor necrosis factor, TNF, TNFA, DIF, TNFSF2, cachexin or cachectin. TNFα is involved in systemic inflammation and is one of the cytokines that comprise the acute phase reaction. TNFα is produced primarily by activated macrophages, but is also produced by CD4+ lymphocytes, natural killer cells, neutrophils, mast cells, eosinophils, and neurons. Non-limiting examples of TNFα amino acid sequences are set forth in GenBank Accession Nos. NM_000594.3→NP_000585.2 (human) and NM_001278601.1→NP_001265530.1 (mouse).

The term “connective tissue growth factor” or “CTGF” refers to the matricellular protein of the CCN family of extracellular matrix-associated heparin binding proteins that is encoded by the CTGF gene. CTGF is also known as CCN2, HCS24, IGFBP8, or NOV2. Non-limiting examples of CTGF amino acid sequences are set forth in GenBank Accession Nos. NM_001901.2→NP_001892.1 (human) and NM_010217.2→NP_034347.2 (mouse). CTGF is associated with virtually all fibrotic pathology, in addition to wound healing. It has also been shown that CTGF cooperates with TGFβ to promote sustained fibrosis.

The term “platelet derived growth factor receptor beta” or “PDGFRβ” refers to the beta form of the platelet derived growth factor receptor that is encoded by the gene PDGFRB. Platelet derived growth factor receptor beta is also known as PDGFRB, CD140B, IBGC4, IMF1, JTK12, PDGFR, PDGFR-1, PDGFR1, KOGS, or PENTT. PDGFRβ is a cell surface tyrosine kinase receptor that, when activated following binding of a PDGF ligand and subsequent dimerization with another PDGFR beta receptor or a PDGFR alpha receptor, activates cellular signaling pathways that play roles in cell proliferation, differentiation, and growth. A non-limiting example of a PDGFRβ amino acid sequence is set forth in GenBank Accession No. NM_002609.3→NP_002600.1. PDGFRβ activation is associated with the replication, survival and migration of myofibroblasts during the progression of fibrotic diseases.

The term “aspartate aminotransferase” or “AST” refers to a pyridoxal phosphate (PLP)-dependent transaminase enzyme (Enzyme Commission number 2.6.1.1) that is also known as aspartate transaminase, AspAT, ASA, AAT, or serum glutamic oxaloacetic transaminase (SGOT). AST plays important roles in amino acid metabolism, catalyzing the transfer of alpha amino groups between aspartate and glutamate. AST is a common biochemical marker of liver disease, as it is released from liver cells following liver injury, manifesting as elevated AST concentrations when measured using a blood test. Normal AST reference ranges for blood tests are 8-40 IU/L for males and 6-34 IU/L for females. The ratio of AST to ALT is also a common clinical biomarker for liver disease.

The term “alanine aminotransferase” or “ALT” refers to a transaminase enzyme (Enzyme Commission number 2.6.1.2) that is also known as alanine transaminase, serum glutamate-pyruvate transaminase (SGPT), or serum glutamic-pyruvate transaminase (SGPT).

ALT catalyzes the transfer of an amino group from L-alanine to α-ketoglutarate and plays important roles in the alanine cycle. ALT is a common biochemical marker of liver disease, as it is released from liver cells following liver injury, manifesting as elevated ALT concentrations when measured using a blood test. Normal ALT reference ranges for blood tests are ≤52 IU/L for males and ≤34 IU/L for females. The ratio of AST to ALT is also a common clinical biomarker for liver disease.

The term “AST to platelet ratio index” or “APRI” refers to a method of using a subject's AST level, as measured using a blood test, and the subject's platelet count to predict the amount of liver fibrosis in the subject, as non-invasive alternative to liver biopsy. APRI is calculated using the following formula:

$$APRI = \frac{\frac{AST level}{AST upper limit of normal}}{platelet count} \times 100$$

wherein AST level and AST upper limit of normal are expressed in units of IU/L and platelet count is expressed in units of 10⁹/L. A commonly-recommended value of AST upper limit of normal is 40 IU/L. Higher APRI values are associated with greater positive predictive values of liver fibrosis.

The term “gamma-glutamyl transferase” or “GGT” refers to an enzyme that transfers gamma-glutamyl functional groups and is also known as gamma-glutamyl transpeptidase, GGTP or gamma-GT (Enzyme Commission number 2.3.2.2). GGT catalyzes the transfer of the gamma-glutamyl moiety of glutathione to acceptors that include amino acids, peptides, and water (i.e., the formation of glutamate), and plays a role in the gamma-glutamyl cycle, which functions in glutathione degradation and drug detoxification. GGT is useful for determining whether an increase in alkaline phosphatase is due to skeletal disease (in which case GGT levels will be normal) or liver disease (in which case GGT will be elevated).

The term “alkaline phosphatase” or “AP” or “ALP” refers to the hydrolase enzyme (Enzyme Commission number 3.1.3.1) that is also known as alkaline phosphomonoesterase, phosphomonoesterase, glycerophosphatase, alkaline phosphohydrolase, alkaline phenyl phosphatase, or orthophosphoric-monoester phosphohydrolase (alkaline optimum). AP removes phosphate groups from many different molecules, including nucleotides, proteins, and alkaloids. When liver cells are damaged, AP is released, thus elevated levels of AP in blood tests can be indicative of liver disease.

The term “bilirubin” refers to the yellow breakdown product of normal heme catabolism, and has a chemical formula of C₃₃H₃₆N₄O₆ and a molar mass of 584.67 g/mol. Measurement of bilirubin can be “indirect” (i.e., unconjugated bilirubin) or “direct” (i.e., conjugated bilirubin). Normal bilirubin levels, when measured using a blood test, range between 0 and 0.3 mg/dl for conjugated bilirubin, and 0.3 to 1.9 mg/dl for total bilirubin (i.e., conjugated and unconjugated bilirubin combined). Bilirubin is excreted from the liver into the bile duct and stored in the gallbladder, and is released into the small intestine as bile to aid digestion. When liver function is impaired, bilirubin is not adequately removed from the blood, resulting in elevated bilirubin levels.

The term “ferritin” refers to a hollow globular protein having a molecular weight of 450 kDa and consisting of 24 subunits that functions to store iron in a non-toxic form and transport and release iron to areas where iron is needed. The light type ferritin subunit is encoded by the FTL gene, and the heavy type subunit is encoded by FTH1 gene (also known as FTHL6). Non-limiting examples of ferritin amino acid sequences are set forth in GenBank Accession Nos. NM_000146.3→NP_000137.2 (light chain) and NM_002032.2→NP_002023.2 (heavy chain). Ferritin is stored in many types of cells, including liver cells. When liver cells are damaged, ferritin is released, resulting in elevated serum ferritin levels. Serum ferritin levels greater than 300 ng/mL in men and 200 ng/mL in women are commonly considered to be abnormal.

The term “fibroblast growth factor 21” or “FGF21” refers to a protein that is encoded by the FGF21 gene in mammals. FGF21 is an important metabolic regulator and plays several roles, including controlling AMP activation and insulin sensitivity. FGF21 stimulates glucose uptake in adipocytes, which is an additive effect to that of insulin. In particular, FGF21 levels are increased in patients who have type 2 diabetes. A non-limiting example of an FGF21 amino acid sequence in humans is set forth under GenBank Accession number NM_019113.3→NP_061986.1.

The term “FGFR1c” refers to a cognate receptor of FGF21. In humans, FGFR1c is expressed from the FGFR1 gene. A non-limiting example of an FGFR1c amino acid sequence in humans is set forth under GenBank Accession number NM_001174063.1→NP_001167534.1.

The term “Beta-klotho” refers to a protein encoded by the KLB gene in humans that increases the ability of FGF21 to bind to FGFR1 (e.g., FGFR1c) and FGFR4. In particular, Beta-klotho, FGF21, and the FGF21 receptor must all be present in a binding complex in order for proper activation of the FGF21 receptor by FGF21 to occur. A non-limiting example of a human amino acid sequence is set forth under GenBank Accession number NM_175737.3→NP_783864.1.

The term “percent identity” or “percent sequence identity,” in the context of describing two or more polynucleotide or amino acid sequences, refers to two or more sequences or subsequences that are the same or have a specified percentage of amino acid residues or nucleotides that are the same (for example, a miR-22 inhibitor). When a peptide or polynucleotide has at least about 70% sequence identity, preferably at least about 80%, 81%, 82%, 83%, 84%, 85%, 86%, 87%, 88%, 89%, 90%, 91%, 92%, 93, 94%, 95%, 96%, 97%, 98%, 99%, or 100% identity, to a reference sequence, when compared and aligned for maximum correspondence over a comparison window, or designated region as measured using one of the following sequence comparison algorithms or by manual alignment and visual inspection, such sequences are then said to be “substantially identical.” With regard to polynucleotide sequences, this definition also refers to the complement of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are used.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Additional examples of algorithms that are suitable for determining percent sequence identity and sequence similarity are the BLAST and BLAST 2.0 algorithms, which are described in Altschul et al., (1990) J. Mol. Biol. 215: 403-410 and Altschul et al. (1977) Nucleic Acids Res. 25: 3389-3402, respectively. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see, e.g., Henikoff and Henikoff, Proc. Natl. Acad. Sci. USA 89:10915 (1989)).

The BLAST algorithm also performs a statistical analysis of the similarity between two sequences (see, e.g., Karlin and Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### III. Methods for Preventing or Treating Metabolic Diseases

In one aspect, the present invention provides a method for preventing or treating a metabolic disease in a subject (e.g., a subject in need thereof). In some embodiments, the method comprises administering (e.g., a therapeutically effective amount) of a miR-22 inhibitor. Depending on the subject, any number of variants of miR-22 may be inhibited. In some embodiments, the miR-22 inhibitor is a human miR-22 (hsa-miR-22) inhibitor.

In some embodiments, the miR-22 inhibitor is an oligonucleotide. In some embodiments, the oligonucleotide is an antisense oligonucleotide. In some embodiments, the oligonucleotide comprises a nucleic acid sequence that has at least about 70% identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:1. In some embodiments, the oligonucleotide comprises the nucleic acid sequence set forth in SEQ ID NO:1.

In some embodiments, the oligonucleotide is at least partially complementary to a miR-22 sequence (e.g., the nucleic acid sequence set forth in SEQ ID NO:2). In some embodiments, the oligonucleotide specifically binds to a nucleic acid sequence having at least about 70% identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:2.

In some embodiments, a miR-22 inhibitor (e.g. an oligonucleotide) used in carrying out the present invention is expressed from a virus. In particular embodiments, a virus containing a nucleotide sequence encoding the miR-22 inhibitor is introduced into a cell (e.g., target cell), and then the miR-22 inhibitor is expressed within the cell. One of ordinary skill in the art will understand that the choice of viral delivery system will depend on the particular indication, target cell type, etc. Non-limiting examples of suitable viruses for expression of miR-22 inhibitors include adenovirus, retrovirus, vaccinia virus, poxvirus, adeno-associated virus, herpes simplex virus, and lentivirus. In some embodiments, the miR-22 inhibitor is expressed from an adenovirus. In some embodiments, a polynucleotide comprising a nucleotide sequence encoding the miR-22 inhibitor is introduced into a cell by another method (e.g., lipofection) or as a naked DNA molecule or plasmid.

In some embodiments, the method further comprises administering (e.g., a therapeutically effective amount) a metabolism-enhancing agent. Non-limiting examples of metabolism-enhancing agents include fibroblast growth factor 21 (FGF21), retinoids, histone deacetylase (HDAC) inhibitors, metformin, bile acids (including synthetic variants) or analogs thereof, resistant starches, prebiotic agents, and probiotic agents. Furthermore, any combination of metabolism-enhancing agents can be used.

As non-limiting examples, retinoids that can be used in methods of the present invention include retinoic acid (RA), retinol, retinal, isotretinoin, alltretinoin, etretinate, acitretin, tazarotene, bexarotene, adapalene, seletinoid G, a retinyl ester, fenretinide, derivatives thereof, and any combination thereof. Suitable retinyl esters include retinyl acetate, retinyl butyrate, retinyl propionate, retinyl palmitate, and any combination thereof. In particular embodiments, the retinoid is RA.

In some embodiments, the HDAC inhibitor is a short-chain fatty acid and/or suberanilohydroxamic acid (SAHA). In particular embodiments, the metabolism-enhancing agent (e.g., retinoid or HDAC inhibitor) is a derivative (e.g., enantiomer) thereof or a prodrug thereof (e.g., tributyrin).

In some embodiments, the HDAC inhibitor that is used is SAHA. In some embodiments, the HDAC inhibitor is an SCFA. Suitable SCFAs include, but are not limited to, propionate, butyrate, isobutyrate, valerate, isovalerate, and any combination thereof. In particular embodiments, the SCFA is butyrate, propionate, and/or valerate. Any other HDAC inhibitor described herein or known to one of skill in the art can be used.

In general, any bile acid receptor (FXR) agonist can be used a metabolism-enhancing agent. In some embodiments, the bile acid receptor agonist is a bile acid, a synthetic variant thereof, or an analog thereof. Suitable bile acids, synthetic variants thereof, or analogs thereof include, but are not limited to, obeticholic acid (OCA; also known as INT-747) and chenodeoxycholic acid (CDCA). OCA is a semi-synthetic bile acid, which has the structure 6a-ethyl-chenodeoxycholic acid. It is known that OCA activates FXR and improves lipid and carbohydrate metabolism.

Furthermore, agonists of G protein-coupled bile acid receptor (GPBAR1), also known as TGR5, can be used as metabolism-enhancing agents, as can dual FXR/TGR5 agonists. Non-limiting examples of dual FXR/TGR5 agonists include INT-747, INT-767, and INT-777.

INT-747 has the following structure:

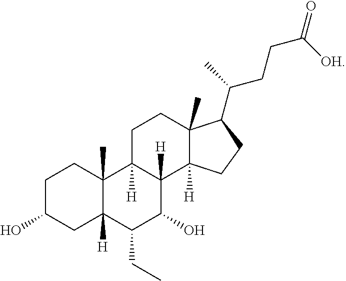

INT-767 has the following structure:

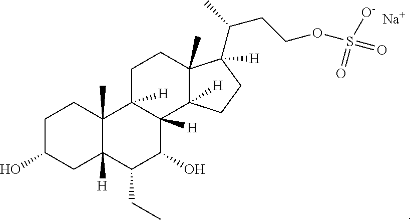

INT-777 has the following structure:

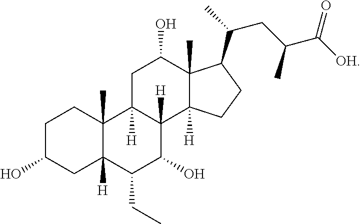

In some embodiments, the metabolism-enhancing agent comprises a probiotic agent and/or a prebiotic agent. In some embodiments, the probiotic agent comprises a bacterium that produces an SCFA. In some embodiments, the prebiotic agent comprises apple pectin and/or an inulin.

Metabolism-enhancing agents can be administered by any suitable route, including but not limited to oral, intravenous, intraperitoneal, intramuscular, subcutaneous, and intranasal administration. In some embodiments, the metabolism-enhancing agent (e.g., FGF21) is virally expressed, e.g., from a suitable viral delivery system described herein such as adenovirus.

In any of the methods described herein, in some embodiments, the method further comprises administering a delivery-enhancing agent to the subject. Non-limiting examples of suitable delivery-enhancing agents include cyclodextrins, an inactivated bacterium, polyvinyl alcohol (PVA), an inulin or ester thereof, and a combination thereof. Suitable inulin esters include, but are not limited to, an inulin butyrate ester, an inulin propionate ester, and a combination thereof. In some embodiments, compositions of the present invention are delivered as a nanoemulsion.

In some embodiments, two or more miR-22 inhibitors and/or metabolism-enhancing agents are combined in a ratio of about 1:10, 1:20, 1:30, 1:40, 1:50, 1:60, 1:70, 1:80, 1:90, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1,000 by weight.

Methods of the present invention are useful for preventing or treating any number of metabolic diseases. In some embodiments, a method or composition of the present invention is used to prevent or treat obesity. In some embodiments, a method or composition of the present invention is used to prevent or treat diabetes (e.g., type 2 diabetes). In particular embodiments, a method or composition of the present invention is used to increase insulin sensitivity. In some embodiments, a method or composition of the present invention is used to prevent or treat obesity. In some embodiments, a method or composition of the present invention is used to prevent or treat dyslipidemia. In some embodiments, a method or composition of the present invention is used to prevent or treat NAFLD or NASH. Fatty liver disease (FLD), also known simply as fatty liver or hepatic steatosis, is a condition wherein large vacuoles of triglyceride fat accumulate in hepatocytes via the process of steatosis (i.e., infiltration of liver cells with fat). FLD can occur in individuals who consume little or no alcohol, in which case the disease is known as non-alcoholic fatty liver disease (NAFLD). The accumulation of fat in the liver leads to inflammation and the development of fibrosis within the liver. As the extent of liver fibrosis increases, the development of more severe non-alcoholic steatohepatitis (NASH) occurs. Accompanying the progression of liver fibrosis due to NAFLD and NASH is a progressive deterioration of liver function, possibly leading to liver failure. FLD is estimated to affect about 10 to 20 percent of Americans, with an additional about 2 to 5 percent being affected by the more severe NASH. NASH is often first suspected in an individual who is found to have elevated levels of one or more biomarkers of liver disease (e.g., ALT and AST), particularly when there is no other apparent reason for liver disease (e.g., heavy alcohol intake, medication, or infection such as hepatitis). A suspicion of NASH may also occur when X-ray or other imaging studies show evidence of fatty liver. The gold standard for distinguishing NASH from more benign FLD is to perform a liver biopsy. Suitable biomarkers for the detection and monitoring of liver disease, including NAFLD and NASH, include but are not limited to aspartate aminotransferase (AST), alanine aminotransferase (ALT), the ratio of AST to ALT (i.e., the AST/ALT ratio is often greater than 2 in progressive NASH), gamma-glutamyl transferase (GGT), the aspartate to platelet ratio index (APRI), alkaline phosphatase (AP), bilirubin, and ferritin.

In particular embodiments, a sample (e.g., test sample) is obtained from the subject. The sample can be obtained before and/or after the miR-22 inhibitor, metabolism-enhancing agent, and/or pharmaceutical composition is administered to the subject. Non-limiting examples of suitable samples include blood, serum, plasma, cerebrospinal fluid, tissue, saliva, urine or any combination thereof. In some instances, the sample comprises normal tissue. In other instances, the sample comprises diseased tissue. The sample can also be made up of normal and/or diseased tissue. Tissue samples can be obtained by biopsy or surgical resection.

In some embodiments, a reference sample is obtained. The reference sample can be obtained, for example, from the subject and can comprise normal tissue. The reference sample can be also be obtained from a different subject and/or a population of subjects. In some instances, the reference sample is either obtained from the subject, a different subject, or a population of subjects before and/or after the miR-22 inhibitor, metabolism-enhancing agent, and/or pharmaceutical composition is administered to the subject, and comprises normal tissue. However, in some instances the reference sample comprises diseased tissue and is obtained from the subject and/or from a different subject or a population of subjects.

In some embodiments, the level and/or activity of one or more biomarkers is determined in the sample (e.g., test sample) and/or reference sample. Non-limiting examples of suitable biomarkers include miRs such as miR-22 (e.g., SEQ ID NO:2). In some embodiments, at least one of the biomarkers is a miR. Other non-limiting examples of suitable biomarkers include fibroblast growth factor 21 (FGF21), fibroblast growth factor receptor 1c (FGFR1c), sirtuin 1 (SIRT1), Beta-klotho, blood glucose, total cholesterol, low-density lipoprotein (LDL), high-density lipoprotein (HDL), triglyceride level, C-reactive protein, hemoglobin A1c, 5′-adenosine monophosphate-activated protein kinase (AMPK), peroxisome proliferator-activated receptor alpha (PPAR-α), peroxisome proliferator-activated receptor gamma coactivator 1-alpha (PGC-1α), aspartate aminotransferase (AST), alanine aminotransferase (ALT), the ratio of AST to ALT, gamma-glutamyl transferase (GGT), the aspartate to platelet ratio index (APRI), alkaline phosphatase (AP), bilirubin, albumin, ferritin, collagen 1a1, cyclin A2, alpha-smooth muscle actin (αSMA), procollagen α1 (procol1), transforming growth factor-β (TGFβ), monocyte chemoattractant protein-1 (MCP1), interleukin-6 (IL-6), interleukin-10 (IL-10), interleukin-17 (IL-17), interleukin-1β (IL-1b), tumor necrosis factor alpha (TNFα), interferon-gamma (INF-γ), RAR-related orphan receptor gamma (ROR-γ), alpha-actin 1 (ACTA1), tissue growth factor beta (TGFβ), connective tissue growth factor (CTGF), platelet derived growth factor receptor beta (PDGFRβ), carnitine palmitoyltransferase 1 (CPT1), 3-hydroxy-3-methylglutaryl-CoA synthase 2 (mitochondrial) (HMGCS2), ERK1, ERK2, phosphoenolpyruvate carboxykinase (PEPCK), glucose-6-phosphatase (G6pase), a fatty acid binding protein (FAPB), fatty acid synthase (FASN), sterol regulatory element-binding protein 1 (SREBP1), cytochrome P450 4A11 (CYP4A11), glucagon-like peptide 1 (GLP1), eptide YY (PYY; also known as peptide tyrosine tyrosine), zona occludens protein 1 (ZO-1), zona occludens protein 2 (ZO-2), zona occludens protein 3 (ZO-3), junctional adhesion molecule 1 (JAM-1), waist circumference, body mass index (BMI), homeostatic model assessment (HOMA) score, and a combination thereof. Any combination of biomarkers, including those described herein and others that will readily be known to one of skill in the art, can be used.

Typically, the level of the one or more biomarkers in one or more samples (e.g., test samples) is compared to the level of the one or more biomarkers in one or more reference samples. The reference sample(s) can be obtained from the subject (e.g., the subject being administered a miR-22 inhibitor or a combination of a miR-22 inhibitor and a metabolism-enhancing agent to prevent or treat a metabolic disease), or can be obtained from a different subject or a population of subjects. As a non-limiting example, the levels of one or more biomarkers in samples (e.g., test samples) taken before and/or after the miR-22 inhibitor, metabolism-enhancing agent, and/or pharmaceutical composition is administered to the subject can be compared to the level of the one or more biomarkers in a normal reference sample (e.g., blood or tissue) that is either obtained from the subject or obtained from a different subject or a population of subjects. In some instances, the biomarker in a test sample obtained from the subject before the subject is treated is lower than the level of the biomarker in the reference sample. In other instances, the level of biomarker in a test sample obtained from the subject after the subject is treated is increased relative to the level of the biomarker in a test sample obtained prior to administration.

In some embodiments, the level and/or activity of FGF21 is decreased in a sample (e.g., a blood sample) obtained from the subject after administration of the miR-22 inhibitor or combination of the miR-22 inhibitor and the metabolism-enhancing agent, compared to a sample (e.g., a blood sample) obtained before administration of the miR-22 inhibitor or combination of the miR-22 inhibitor and the metabolism-enhancing agent. In some embodiments, the level and/or activity of FGF21 is increased in a sample (e.g., a liver tissue sample) obtained from the subject after administration of the miR-22 inhibitor or combination of the miR-22 inhibitor and the metabolism-enhancing agent, compared to a sample (e.g., liver tissue sample) obtained before administration of the miR-22 inhibitor or combination of the miR-22 inhibitor and the metabolism-enhancing agent. In some embodiments, the level and/or activity of FGFR1, AMPK, and/or Beta-klotho is increased in a sample (e.g., a liver tissue sample) obtained from the subject after administration of the miR-22 inhibitor or combination of the miR-22 inhibitor and the metabolism-enhancing agent, compared to a sample (e.g., liver tissue sample) obtained before administration of the miR-22 inhibitor or combination of the miR-22 inhibitor and the metabolism-enhancing agent.

In some embodiments, the level and/or activity of miR-22 is increased in a sample (e.g., a blood sample) obtained from the subject after administration of the miR-22 inhibitor or combination of the miR-22 inhibitor and the metabolism-enhancing agent, compared to a sample (e.g., a blood sample) obtained before administration of the miR-22 inhibitor or combination of the miR-22 inhibitor and the metabolism-enhancing agent. In some embodiments, the level and/or activity of miR-22 is decreased in a sample (e.g., a liver tissue sample) obtained from the subject after administration of the miR-22 inhibitor or combination of the miR-22 inhibitor and the metabolism-enhancing agent, compared to a sample (e.g., a liver tissue sample) obtained before administration of the miR-22 inhibitor or combination of the miR-22 inhibitor and the metabolism-enhancing agent.

From the disclosure provided herein, it is also understood that various biomarkers, including miR-22, are useful for the diagnosis of metabolic diseases. For example, a decreased level of serum miR-22 and/or an increased level of hepatic miR-22 can be used to indicate the presence or increased risk of a metabolic disease. Furthermore, a decreased serum miR-22 level and/or an increased hepatic miR-22 level can be used to quantify the severity of a metabolic disease. In some embodiments, a decreased serum miR-22 level and/or an increased hepatic miR-22 level indicates that a subject has a fatty liver.

The differences between the reference sample or value and the sample (e.g., test sample) need only be sufficient to be detected. In some embodiments, a decreased level of a biomarker in the test sample, and hence the presence of a metabolic disease or the risk of a metabolic disease, is determined when the biomarker levels are at least, e.g., 10%, 25%, 50% or more lower in comparison to a negative control. In some embodiments, an increased level of a biomarker in the test sample, and hence the presence of a metabolic disease or the risk of a metabolic disease, is determined when the biomarker levels are at least, e.g., 10%, 25%, 50% or more greater in comparison to a negative control.

The biomarker levels can be detected using any method known in the art, including the use of PCR (e.g., quantitative PCR), nucleic acid sequencing, and various protein detection and quantification methods, including those that utilize antibodies specific for the biomarkers. Exemplary methods include, without limitation, Western Blot, dot blot, enzyme-linked immunosorbent assay (ELISA), radioimmunoassay (RIA), immunoprecipitation, immunofluorescence, FACS analysis, electrochemiluminescence, and multiplex bead assays (e.g., using Luminex® or fluorescent microbeads).

In some embodiments, the antibody or plurality thereof used to detect the biomarker(s) can be immobilized on a solid support. The solid support can be, for example, a multiwell plate, a microarray, a chip, a bead, a porous strip, or a nitrocellulose filter. In some instances, the bead comprises chitin. The immobilization can be via covalent or non-covalent binding.

Labeled secondary antibodies can be used to detect binding between antibodies and biomarkers. Secondary antibodies bind to the constant or “C” regions of different classes or isotypes of immunoglobulins IgM, IgD, IgG, IgA, and IgE. Usually, a secondary antibody against an IgG constant region is used in the present methods. Secondary antibodies against the IgG subclasses, for example, IgG1, IgG2, IgG3, and IgG4, also find use in the present methods. Secondary antibodies can be labeled with any directly or indirectly detectable moiety, including a fluorophore (e.g., fluorescein, phycoerythrin, quantum dot, Luminex® bead, fluorescent bead), an enzyme (e.g., peroxidase, alkaline phosphatase), a radioisotope (e.g., ³H, ³²P, ¹²⁵I) or a chemiluminescent moiety. Labeling signals can be amplified using a complex of biotin and a biotin binding moiety (e.g., avidin, streptavidin, neutravidin). Fluorescently labeled anti-human IgG antibodies are commercially available from Molecular Probes, Eugene, Oreg. Enzyme-labeled anti-human IgG antibodies are commercially available from Sigma-Aldrich, St. Louis, Mo. and Chemicon, Temecula, Calif.

General immunoassay techniques are well known in the art. Guidance for optimization of parameters can be found in, for example, Wu, Quantitative Immunoassay: A Practical Guide for Assay Establishment, Troubleshooting, and Clinical Application, 2000, AACC Press; Principles and Practice of Immunoassay, Price and Newman, eds., 1997, Groves Dictionaries, Inc.; The Immunoassay Handbook, Wild, ed., 2005, Elsevier Science Ltd.; Ghindilis, Pavlov and Atanassov, Immunoassay Methods and Protocols, 2003, Humana Press; Harlow and Lane, Using Antibodies: A Laboratory Manual, 1998, Cold Spring Harbor Laboratory Press; and Immunoassay Automation: An Updated Guide to Systems, Chan, ed., 1996, Academic Press.

In certain embodiments, the presence or decreased or increased presence of one or more biomarkers is indicated by a detectable signal (e.g., a blot, fluorescence, chemiluminescence, color, radioactivity) in an immunoassay. This detectable signal can be compared to the signal from a control sample or to a threshold value. In some embodiments, decreased presence is detected, and the presence or increased risk of metabolic disease is indicated, when the detectable signal of biomarker(s) in the test sample is at least about 10%, 20%, 30%, 50%, 75% lower in comparison to the signal of antibodies in the reference sample or the predetermined threshold value. In other embodiments, an increased presence is detected, and the presence or increased risk of metabolic disease is indicated, when the detectable signal of biomarker(s) in the test sample is at least about 1-fold, 2-fold, 3-fold, 4-fold or more, greater in comparison to the signal of antibodies in the reference sample or the predetermined threshold value.

In some embodiments, the results of the biomarker level determinations are recorded in a tangible medium. For example, the results of diagnostic assays (e.g., the observation of the presence, decreased presence or increased presence of one or more biomarkers) and the diagnosis of whether or not there is an increased risk or the presence of a metabolic disease can be recorded, e.g., on paper or on electronic media (e.g., audio tape, a computer disk, a CD, a flash drive, etc.).

In some embodiments, administering a miR-22 inhibitor, metabolism-enhancing agent, and/or a composition described herein improves one or more signs or symptoms of a metabolic disease (e.g., in a subject). In some embodiments, administering the miR-22 inhibitor, metabolism-enhancing agent, or composition described herein improves insulin sensitivity (e.g., in a subject).

In other embodiments, the methods further comprise the step of providing a diagnosis and/or prognosis to the patient (i.e., the subject) and/or the results of treatment.

### IV. Compositions and Administration

In another aspect, the present invention provides a pharmaceutical composition. In some embodiments, the pharmaceutical composition comprises a miR-22 inhibitor and/or a metabolism-enhancing agent, and a pharmaceutically acceptable carrier. In some embodiments, the pharmaceutical composition comprises a miR-22 inhibitor, a metabolism-enhancing agent and a pharmaceutically acceptable carrier. In some embodiments, the pharmaceutical composition comprises a therapeutically effective amount of the miR-22 inhibitor and/or the metabolism-enhancing agent. In some embodiments, the miR-22 is a human miR-22 (hsa-miR-22) inhibitor.

The pharmaceutical compositions described herein are useful for the prevention or treatment of a metabolic disease. Non-limiting examples of suitable metabolic diseases include alcoholic steatohepatitis (ASH), non-alcoholic steatohepatitis (NASH), non-alcoholic fatty liver disease (NAFLD), diabetes (e.g., type 2 diabetes), obesity, dyslipidemia, and a combination thereof.

In some embodiments, the miR-22 inhibitor is an oligonucleotide. In some embodiments, the oligonucleotide is an antisense oligonucleotide. In some embodiments, the oligonucleotide comprises a nucleic acid sequence that has at least about 70% identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:1. In some embodiments, the oligonucleotide comprises the nucleic acid sequence set forth in SEQ ID NO:1.

In some embodiments, the oligonucleotide is at least partially complementary to a miR-22 sequence (e.g., the nucleic acid sequence set forth in SEQ ID NO:2). In some embodiments, the oligonucleotide specifically binds to a nucleic acid sequence having at least about 70% identity (e.g., at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to SEQ ID NO:2.

Non-limiting examples of metabolism-enhancing agents that can be used in pharmaceutical compositions of the present invention include fibroblast growth factor 21 (FGF21), retinoids, histone deacetylase (HDAC) inhibitors, metformin, bile acids (including synthetic variants) or analogs thereof, resistant starches, prebiotic agents, and probiotic agents. Furthermore, any combination of metabolism-enhancing agents can be used.

As non-limiting examples, retinoids that can be used in compositions of the present invention include retinoic acid (RA), retinol, retinal, isotretinoin, alltretinoin, etretinate, acitretin, tazarotene, bexarotene, adapalene, seletinoid G, a retinyl ester, fenretinide, derivatives thereof, and any combination thereof. Suitable retinyl esters include retinyl acetate, retinyl butyrate, retinyl propionate, retinyl palmitate, and any combination thereof. In particular embodiments, the retinoid is RA.

In some embodiments, the concentration of the retinoid (e.g., RA) is about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 μM, or more. In particular embodiments, the concentration of the retinoid (e.g., RA) is about 10 μM.

In some embodiments, the HDAC inhibitor is a short-chain fatty acid and/or suberanilohydroxamic acid (SAHA). In particular embodiments, the metabolism-enhancing agent (e.g., retinoid or HDAC inhibitor) is a derivative (e.g., enantiomer) thereof or a prodrug thereof (e.g., tributyrin).

In some embodiments, the HDAC inhibitor that is used is SAHA. In some embodiments, the HDAC inhibitor is an SCFA. Suitable SCFAs include, but are not limited to, propionate, butyrate, isobutyrate, valerate, isovalerate, and any combination thereof. In particular embodiments, the SCFA is butyrate, propionate, and/or valerate. Any other HDAC inhibitor described herein or known to one of skill in the art can be used.

In some embodiments, the concentration of SAHA is about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 μM, or more. In particular embodiments, the concentration of SAHA is about 5 μM.

In some embodiments, the concentration of the SCFA is about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 mM, or more. In particular embodiments, the concentration is about 5 mM. In some embodiments, the concentration of butyrate, propionate, and/or valerate is about 5 mM. In some embodiments, the concentration of butyrate, propionate, and/or valerate is about 10 mM.

In general, any bile acid receptor (FXR) agonist can be used a metabolism-enhancing agent. In some embodiments, the bile acid receptor agonist is a bile acid, a synthetic variant thereof, or an analog thereof. Suitable bile acids, synthetic variants thereof, or analogs thereof include, but are not limited to, obeticholic acid (OCA; also known as INT-747) and chenodeoxycholic acid (CDCA). OCA is a semi-synthetic bile acid, which has the structure 6a-ethyl-chenodeoxycholic acid. It is known that OCA activates FXR and improves lipid and carbohydrate metabolism.

Furthermore, agonists of G protein-coupled bile acid receptor (GPBAR1), also known as TGR5, can be used as metabolism-enhancing agents, as can dual FXR/TGR5 agonists. Non-limiting examples of dual FXR/TGR5 agonists include INT-747, INT-767, and INT-777.

INT-747 has the following structure:

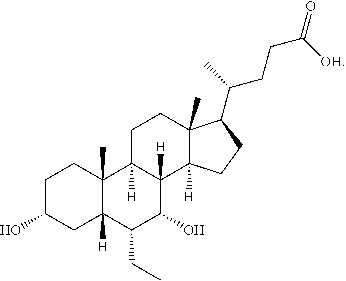

INT-767 has the following structure:

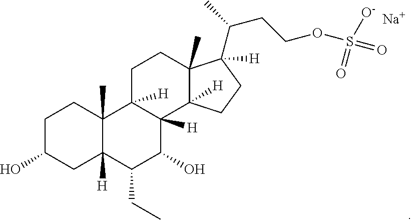

INT-777 has the following structure:

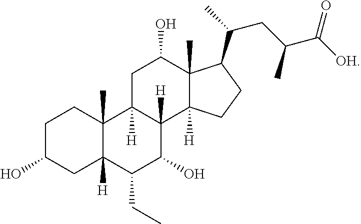

In some embodiments, a pharmaceutical composition described herein comprises a nanoemulsion. In some embodiments, the metabolism-enhancing agent comprises a probiotic agent and/or a prebiotic agent. Suitable prebiotic agents include, but are not limited to, apple pectin, inulin (or an ester thereof), and a combination thereof. In some instances, a probiotic agent is a bacterium that produces an SCFA (e.g., butyrate, propionate) such as Roseburia hominis or Propionibacterium freudenreichii.

Pharmaceutical compositions or medicaments for use in the present invention can be formulated by standard techniques using one or more physiologically acceptable carriers or excipients. Suitable pharmaceutical carriers are described herein and in “Remington's Pharmaceutical Sciences” by E. W. Martin. Compounds and agents of the present invention and their physiologically acceptable salts and solvates can be formulated for administration by any suitable route, including via inhalation, topically, nasally, orally, intravenously, parenterally, or rectally.

In some embodiments, a pharmaceutical composition of the present invention further comprises a delivery-enhancing agent. In some embodiments, the delivery-enhancing agent comprises a cyclodextrin. Cyclodextrins, which are a family of compounds that comprise cyclic oligosaccharides, can take the form of alpha-cyclodextrins (having a 6-membered ring), beta-cyclodextrins (having a 7-membered ring), or gamma cyclodextrins (having an 8-membered ring). Cyclodextrins can increase the aqueous solubility of compounds and can increase bioavailability and stability. Folate-conjugated amphiphilic cyclodextrins and derivatives thereof can be used for tumor targeting. Polycationic amphiphilic cyclodextrins enhance the interaction of compounds with cell mebranes. Non-limiting examples of particularly useful cyclodextrins include Captisol® and Dexolve™ (sulfobutyl-ether-beta-cyclodextrin). Captisol® is useful for, among other things, improving the solubility, stability, bioavailability or compounds for administration, as well as decreasing volatility, irritation, smell, or taste.

In some embodiments, a delivery-enhancing agent comprises inactivated bacteria. Encapsulating miR-22 inhibitors and/or metabolism agents described herein into inactivated bacteria is especially useful for oral administration, as the miR-22 inhibitors and/or metabolism-enhancing agents can be delivered to the gut with increased inactivity. This method is further described in PCT Application Publication No. WO/2016/069740, hereby incorporated by reference for all purposes.

In some embodiments, two or more miR-22 inhibitors and/or metabolism-enhancing agents are combined in a ratio of about 1:1, 1:2, 1:3, 1:4, 1:5, 1:6, 1:7, 1:8, 1:9, 1:10, 1:20, 1:30, 1:40, 1:50, 1:60, 1:70, 1:80, 1:90, 1:100, 1:150, 1:200, 1:250, 1:300, 1:350, 1:400, 1:450, 1:500, 1:550, 1:600, 1:650, 1:700, 1:750, 1:800, 1:850, 1:900, 1:950, or 1:1,000 by weight.

In some embodiments, two or more miR-22 inhibitors and/or metabolism-enhancing agents are combined in a ratio between about 1:10 and about 1:100, about 1:50 and about 1:100, about 1:20 and about 1:100, about 1:50 and about 1:200, about 1:100 and about 1:200, about 1:100 and about 1:300, about 1:100 and about 1:400, about 1:100 and about 1:500, about 1:100 and about 1:600, about 1:100 and about 1:700, about 1:100 and about 1:800, about 1:100 and about 1:900, or about 1:500 and about 1:1,000 by weight.

In some embodiments, the delivery-enhancing agent comprises an inulin. Inulins are a class of naturally occurring polysaccharides that belong to a class of dietary fibers known as fructans. In humans, inulins are indigestible, whereas bacterial fermentation can lead to the generation of butyrate and propionate from inulins. Because of their resistance to acids and human digestive enzymes, inulins find utility for oral drug delivery, in particular the delivery of drugs to the colon, where they can be readily absorbed through the gut epithelium. Inulin esters are also useful for methods and compositions of the present invention. Suitable inulin esters include, but are not limited to inulin butyrate esters, inulin propionate esters, and a combination thereof.

In some embodiments, an active agent (e.g., a miR-22 inhibitor and/or metabolism-enhancing agent) in encapsulated (e.g., nanoencapsulated). In some embodiments, the compositions of the present invention comprise active agents that are encapsulated (e.g., with glucosamine butyrate or a glucosamine butyrate-gelatin matrix). In some embodiments, an active agent is encapsulated in a matrix that comprises an emulsifier (e.g., a monoester, diester, or organic ester of a glyceride), a carbohydrate hydrocolloid, an unmodified or modified starch, a pectin, a glucan, a cyclodextrin, a maltodextrin, or a protein (e.g., a casein, whey, or soy).

Furthermore, one or more active agents (e.g., a miR-22 inhibitor and/or metabolism-enhancing agent) can be complexed, e.g., in a liposome, in a nanoparticle, in a supramolecular assembly, or an ion pair.

In some embodiments, a composition of the present invention comprises a Eudragit® polymer. Eudragit® is useful for protecting compounds from being dissolved in the stomach, allowing them to be available for release and in more distal regions of the GI tract. Eudragit® L, S, FS, and E polymers are available with acidic or alkaline groups that allow for pH-dependent drug release. Eudragit® RL and RS polymers (cationic groups) and Eudragit® NM polymer with neutral groups enable the time-release of drugs. Eudragit® is commercially available from Evonik.

Furthermore, encapsulation of active agents or compounds in polymeric micelles, inulins (and esters thereof), nanoparticles, or cross-linked chitosan microspheres are useful for delivery.

For inulin-based delivery (e.g., tablets and capsules), a three-component design can be used, wherein the three components include: (1) a hard gelatin enteric-coated capsule (for carrying two pulses), (2) first-pulse granules (for rapid release in intestine), and (3) second-pulse matrix tablet (for slow release in the colon).

Nanoparticles can be made with EudragitR S100. Alternatively, mucoadhesive nanoparticles can be created with trimethylchitosan (TMC). Also, a mix of polymers (e.g., PLGA, PEG-PLGA, and PEG-PCL) can be used to obtain a sustained drug delivery.

For cross-linked chitosan microspheres, a multiparticulate system comprising pH-sensitive properties and specific biodegradability for colon-targeted delivery of agents such as miR-22 inhibitors and metabolism-enhancing agents can be used. As a non-limiting example, cross-linked chitosan microspheres can be prepared from an emulsion system using liquid paraffin as the external phase and a solution of chitosan in acetic acid as the disperse phase. The multiparticulate system is prepared by coating cross-linked chitosan microspheres exploiting Eudragit® L-100 and S-100 as pH-sensitive polymers. Furthermore, cellulose acetate butyrate (CAB) can be used to enhance colonic delivery.

In some embodiments, a composition of the present invention comprises an active agent (e.g., a miR-22 inhibitor and/or a metabolism-enhancing agent) in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by weight. In some embodiments, the active agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%-100% by weight.

In some embodiments, a composition of the present invention comprises an active agent (e.g., a miR-22 inhibitor and/or a metabolism-enhancing agent) in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by volume. In some embodiments, the active agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%-100% by volume.

In some embodiments, a composition of the present invention comprises an inactive agent (i.e., not a miR-22 inhibitor or a metabolism-enhancing agent) in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by weight. In some embodiments, the inactive agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%-100% by weight.

In some embodiments, a composition of the present invention comprises an inactive agent (i.e., an agent or compound present in the composition that is not a miR-22 inhibitor or a metabolism-enhancing agent) in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by volume. In some embodiments, the inactive agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%-100% by volume.

In pharmaceutical compositions that comprise a delivery enhancing agent, in some embodiments, the delivery-enhancing agent is present in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by weight. In some embodiments, the delivery-enhancing agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%-100% by weight.

In some embodiments, the delivery-enhancing agent is present in an amount that is about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% by volume. In some embodiments, the delivery-enhancing agent is between about 1%-10%, 1%-20%, 1%-30%, 1%-40%, 1%-50%, 1%-60%, 1%-70%, 1%-80%, 1%-90%, 1%-100%, 10%-20%, 10%-30%, 10%-40%, 10%-50%, 10%-60%, 10%-70, 10%-80, 10%-90, 10%-100%, 20%-30%, 20%-40%, 20%-50%, 20%-60%, 20%-70%, 20%-80%, 20%-90%, 20%-100%, 30%-40%, 30%-50%, 30%-60%, 30%-70%, 30%-80%, 30%-90%, 30%-100%, 40%-50%, 40%-60%, 40%-70%, 40%-80%, 40%-90%, 40%-100%, 50%-60%, 50%-70%, 50%-80%, 50%-90%, 50%-100%, 60%-70%, 60%-80%, 60%-90%, 60%-100%, 70%-80%, 70%-90%, 70%-100%, 80%-90%, 80%-100%, or 90%-100% by volume

a. Routes of Administration

Exemplary formulations and routes of administration for the delivery of miR-22 inhibitors and/or metabolism-enhancing agents are described herein.

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral or rectal administration is also contemplated.

Suitable formulations for transdermal application include an effective amount of one or more compositions or compounds described herein, optionally with a carrier. Preferred carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used.

For oral administration, a pharmaceutical formulation or a medicament can take the form of, for example, a tablet or a capsule prepared by conventional means with a pharmaceutically acceptable excipient. The present invention provides tablets and gelatin capsules comprising: (1) a miR-22 inhibitor and/or a metabolism-enhancing agent, alone or in combination with other compounds, or a dried solid powder of these drugs, together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, talcum, stearic acid, magnesium or calcium salt, metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulphate, and/or (f) absorbents, colorants, flavors and sweeteners. In some embodiments, an amorphous solid dispersion of an active agent (e.g., a miR-22 inhibitor and/or a metabolism-enhancing agent) is prepared that is suitable for oral delivery.

Tablets may be either film coated or enteric coated according to methods known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound(s).

The compositions and formulations set forth herein can be formulated for parenteral administration by injection, for example by bolus injection or continuous infusion. Formulations for injection can be presented in unit dosage form, for example, in ampules or in multi-dose containers, with an added preservative. Injectable compositions are preferably aqueous isotonic solutions or suspensions, and suppositories are preferably prepared from fatty emulsions or suspensions. The compositions may be sterilized and/or contain adjuvants, such as preserving, stabilizing, wetting or emulsifying agents, solution promoters, salts for regulating the osmotic pressure and/or buffers. Alternatively, the active ingredient(s) can be in powder form for constitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the active ingredient(s).

For administration by inhalation, the compositions of the present invention may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compositions set forth herein can also be formulated in rectal compositions, for example, suppositories or retention enemas, for example, containing conventional suppository bases, for example, cocoa butter or other glycerides.

Furthermore, the active ingredient(s) can be formulated as a depot preparation. Such long-acting formulations can be administered by implantation (for example, subcutaneously or intramuscularly) or by intramuscular injection. Thus, for example, one or more of the compounds described herein can be formulated with suitable polymeric or hydrophobic materials (for example as an emulsion in an acceptable oil) or ion exchange resins, or as sparingly soluble derivatives, for example, as a sparingly soluble salt.

The therapeutic agent(s) may be used individually, sequentially, or in combination with one or more other such therapeutic agents (e.g., a first therapeutic agent, a second therapeutic agent, a compound of the present invention, etc.). In some embodiments, the miR-22 inhibitor and the metabolism-enhancing agent are co-administered. In some embodiments, the miR-22 inhibitor and the metabolism-enhancing agent are administered sequentially. In some embodiments, the miR-22 inhibitor is administered about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more minutes before the metabolism-enhancing agent is administered, or vice versa. In some embodiments, the miR-22 inhibitor is administered about 1, 2, 3, 4, 5, 6, 7, 8, 9, 10, 11, 12, 13, 14, 15, 16, 17, 18, 19, 20, 21, 22, 23, 24, or more hours before the metabolism-enhancing agent is administered, or vice versa. In some embodiments, the miR-22 inhibitor is administered about 1, 2, 3, 4, 5, 6, 7, or more days before the metabolism-enhancing agent is administered, or vice versa. Administration may be by the same or different route of administration or together in the same pharmaceutical formulation.

b. Dosage

Pharmaceutical compositions or medicaments can be administered to a subject at a therapeutically effective dose to prevent, treat, or control a metabolic disease (e.g., NASH, NAFLD, diabetes (e.g., type 2 diabetes), dyslipidemia, obesity, or a combination thereof), as described herein. The pharmaceutical composition or medicament is administered to a subject in an amount sufficient to elicit an effective therapeutic response in the subject.

The dosage of active agents administered may be dependent on the subject's body weight, age, individual condition, surface area or volume of the area to be treated and on the form of administration. The size of the dose also will be determined by the existence, nature, and extent of any adverse effects that accompany the administration of a particular formulation in a particular subject. A unit dosage for oral administration to a mammal of about 50 to about 70 kg may contain between about 5 and about 500 mg, about 25-200 mg, about 100 and about 1000 mg, about 200 and about 2000 mg, about 500 and about 5000 mg, or between about 1000 and about 2000 mg of an active ingredient. A unit dosage for oral administration to a mammal of about 50 to about 70 kg may contain about 10 mg, 20 mg, 25 mg, 50 mg, 75 mg, 100 mg, 200 mg, 300 mg, 400 mg, 500 mg, 600 mg, 700 mg, 800 mg, 900 mg, 1,000 mg, 1,250 mg, 1,500 mg, 2,000 mg, 2,500 mg, 3,000 mg, or more of an active ingredient. Typically, a dosage of the active compound(s) of the present invention is a dosage that is sufficient to achieve the desired effect. Optimal dosing schedules can be calculated from measurements of active agent accumulation in the body of a subject. In general, dosage may be given once or more of daily, weekly, or monthly. Persons of ordinary skill in the art can easily determine optimum dosages, dosing methodologies and repetition rates.

Optimum dosages, toxicity, and therapeutic efficacy of the compositions of the present invention may vary depending on the relative potency of the administered composition and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Agents that exhibit large therapeutic indices are preferred. While agents that exhibit toxic side effects can be used, care should be taken to design a delivery system that targets such agents to the site of affected tissue to minimize potential damage to normal cells and, thereby, reduce side effects.

Optimal dosing schedules can be calculated from measurements of active ingredient accumulation in the body of a subject. In general, dosage is from about 1 ng to about 1,000 mg per kg of body weight and may be given once or more daily, weekly, monthly, or yearly. Persons of ordinary skill in the art can easily determine optimum dosages, dosing methodologies and repetition rates. One of skill in the art will be able to determine optimal dosing for administration of miR-22 inhibitors and/or metabolism-enhancing agents to a human being following established protocols known in the art and the disclosure herein.

The data obtained from, for example, animal studies (e.g. rodents and monkeys) can be used to formulate a dosage range for use in humans. The dosage of compounds of the present invention lies preferably within a range of circulating concentrations that include the ED₅₀ with little or no toxicity. The dosage can vary within this range depending upon the dosage form employed and the route of administration. For any composition for use in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC). In general, the dose equivalent of a chimeric protein, preferably a composition is from about 1 ng/kg to about 100 mg/kg for a typical subject.

A typical composition of the present invention for oral or intravenous administration can be about 0.1 to about 10 mg of active ingredient per patient per day; about 1 to about 100 mg per patient per day; about 25 to about 200 mg per patient per day; about 50 to about 500 mg per patient per day; about 100 to about 1000 mg per patient per day; or about 1000 to about 2000 mg per patient per day. Exemplary dosages include, but are not limited to, about 10 mg, 20 mg, 25 mg, 50 mg, 75 mg, 100 mg, 200 mg, 300 mg, 400 mg, 500 mg, 600 mg, 700 mg, 800 mg, 900 mg, 1,000 mg, 1,250 mg, 1,500 mg, 2,000 mg, 2,500 mg, 3,000 mg, or more of the active ingredient per patient per day. Methods for preparing administrable compositions will be known or apparent to those skilled in the art and are described in more detail in such publications as Remington: The Science and Practice of Pharmacy, 21^{st} Ed., University of the Sciences in Philadelphia, Lippencott Williams & Wilkins (2005).

Exemplary doses of the compositions described herein include milligram or microgram amounts of the composition per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a composition depend upon the potency of the composition with respect to the desired effect to be achieved. When one or more of these compositions is to be administered to a mammal, a physician, veterinarian, or researcher may, for example, prescribe a relatively low dose at first, subsequently increasing the dose until an appropriate response is obtained. In addition, it is understood that the specific dose level for any particular mammal subject will depend upon a variety of factors including the activity of the specific composition employed, the age, body weight, general health, gender, and diet of the subject, the time of administration, the route of administration, the rate of excretion, any drug combination, and the degree of expression or activity to be modulated.

To achieve the desired therapeutic effect, compounds or agents described herein may be administered for multiple days at the therapeutically effective daily dose. Thus, therapeutically effective administration of compounds to treat a metabolic disease (e.g., obesity, diabetes (e.g., type 2 diabetes), dyslipidemia, NASH, NAFLD, or a combination thereof) in a subject may require periodic (e.g., daily) administration that continues for a period ranging from three days to two weeks or longer. Compositions set forth herein may be administered for at least three consecutive days, often for at least five consecutive days, more often for at least ten, and sometimes for 20, 30, 40 or more consecutive days. While consecutive daily doses are a preferred route to achieve a therapeutically effective dose, a therapeutically beneficial effect can be achieved even if the agents are not administered daily, so long as the administration is repeated frequently enough to maintain a therapeutically effective concentration of the agents in the subject. For example, one can administer the agents every other day, every third day, or, if higher dose ranges are employed and tolerated by the subject, once a week, once every two weeks, once every three weeks, once every four weeks, or even less frequently.

In some cases, the recitation of a dose “per day” refers to the amount of drug administered each day. In other cases, the “per day” dose refers to the average amount per day of drug administered over a period of time. Thus, if a drug is administered once a week at 100 mg, then the “per day” dose would be approximately equal to (100 mg/7 days=) 14.3 mg per day.

Following successful treatment, it may be desirable to have the subject undergo maintenance therapy to prevent the recurrence of the metabolic disease (e.g., obesity, diabetes (e.g., type 2 diabetes), dyslipidemia, NASH, NAFLD, or a combination thereof).

Determination of an effective amount is well within the capability of those skilled in the art, especially in light of the detailed disclosure provided herein. Generally, an efficacious or effective amount of an composition is determined by first administering a low dose or small amount of the composition, and then incrementally increasing the administered dose or dosages, adding a second or third medication as needed, until a desired effect of is observed in the treated subject with minimal or no toxic side effects.

Single or multiple administrations of the compositions are administered depending on the dosage and frequency as required and tolerated by the patient. In any event, the composition should provide a sufficient quantity of the compositions of this invention to effectively treat the patient. Generally, the dose is sufficient to treat, improve, or ameliorate symptoms or signs of disease without producing unacceptable toxicity to the patient.

### V. Kits, Containers, Devices, and Systems

A wide variety of kits, systems, and compositions can be prepared according to the present invention, depending upon the intended user of the kit and system and the particular needs of the user. In some embodiments, the present invention provides a kit that includes a miR-22 inhibitor or a combination of a miR-22 inhibitor and a metabolism-enhancing agent, alone or in combination with other compounds. In some embodiments, the kit contains a pharmaceutical composition (e.g., comprising a miR-22 inhibitor and a metabolism-enhancing agent) of the present invention.

In some embodiments, the present invention provides a kit that includes a container containing a miR-22 inhibitor and a container containing a metabolism-enhancing agent.

The compositions of the present invention, including but not limited to compositions containing a miR-22 inhibitor and a metabolism-enhancing agent, may, if desired, be presented in a bottle, jar, vial, ampoule, tube, or other container-closure system approved by the Food and Drug Administration (FDA) or other regulatory body, which may provide one or more dosages containing the active ingredient. The package or dispenser may also be accompanied by a notice associated with the container in a form prescribed by a governmental agency regulating the manufacture, use, or sale of pharmaceuticals, or the notice indicating approval by the agency. In certain aspects, the kit may include a formulation or composition as taught herein, a container closure system including the formulation or a dosage unit form including the formulation, and a notice or instructions describing a method of use as taught herein.

In some embodiments, the kit includes a container which is compartmentalized for holding the various elements of a formulation (e.g., the dry ingredients and the liquid ingredients) or composition, instructions for making the formulation or composition, and instructions for preventing, treating, or controlling a metabolic disease (e.g., diabetes (e.g., type 2 diabetes), dyslipidemia, obesity, NASH, NAFLD, or a combination thereof) in a subject. In certain embodiments, the kit may include the pharmaceutical preparation in dehydrated or dry form, with instructions for its rehydration (or reconstitution) and administration.

Kits with unit doses of the active composition, e.g. in oral, rectal, transdermal, or injectable doses (e.g., for intramuscular, intravenous, or subcutaneous injection), are provided. In such kits, in addition to the containers containing the unit doses will be an informational package insert describing the use and attendant benefits of the composition in preventing, treating, or controlling a metabolic disease. Suitable active compositions and unit doses are those described herein.

While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

### VI. Examples

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Example 1. miR-22 Diagnostic Assays and miR-22 Inhibitors for the Treatment of Metabolic Conditions

Being overweight and obesity predispose millions of Americans to metabolic diseases including non-alcoholic steatosis (NAFL), non-alcoholic steatohepatitis (NASH), type 2 diabetes mellitus (T2DM), and liver cancer. Population-based studies showed that NAFLD and NASH are more prevalent in men than women. The prevalence of T2DM, as well as liver cancer, is also gender different. The current application aims to develop diagnostic blood test to quantify miR-22 for metabolic disease diagnosis and NASH treatment based on gender.

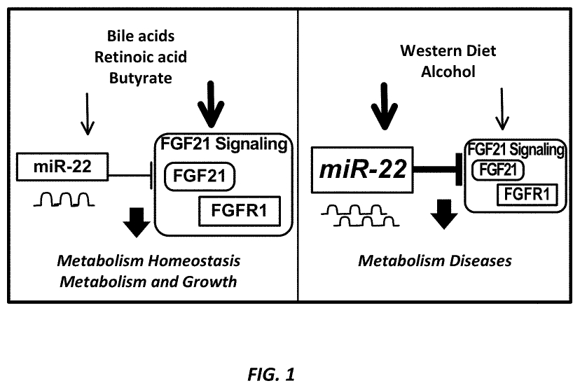

Fibroblast growth factor 21 (FGF21) is a master metabolic regulator for AMPK activation that reverses diabetes and obesity. Although FGF21 can be produced by the liver, adipose tissues, and pancreas, circulating FGF21 is primarily liver-produced. Our data revealed that hepatic and serum FGF21 levels are inversely correlated. In humans, the level of blood FGF21 increases progressively with the advancement of hyperglycemia from prediabetes to diabetes. FGF21 can serve as a biomarker for T2DM risk. In addition, FGF21 signaling pathway is a therapeutic target for T2DM treatment. Although clinical trials of FGF21 mimetic generated promising results with improvement of dyslipidemia, FGF21 resistances, as well as its potential side effects, are of concerns. In effort to improve FGF21 efficacy and identification of additional biomarkers for metabolic conditions, we uncovered that miR-22 reduces FGF21 as well as its receptor FGFR1. Consequently, miR-22 decreases PGC1α and inactivates AMPK. Thus, miR-22 inhibitors should be able to increase FGF21 signaling leading to improved metabolism and reduced inflammation. Although miR-22 is a metabolic inhibitor, our novel data showed that chemicals having metabolic beneficial function such as bile acids, retinoic acid, and butyrate can simultaneously induce both FGF21 as well as miR-22. Thus, we hypothesize that miR-22 is a metabolic restrictor, which prohibits sustained FGF21 activation to maintain metabolism homeostasis (FIG. 1). Moreover, our data revealed that the expression level of miR-22 and FGF21 is gender different, which may explain gender difference in metabolism as well as susceptibility to metabolic disease. Due to the significant role of miR-22 in regulating metabolism, this project establishes a clinical test that quantifies blood miR-22 for NAFL and NASH diagnosis.

Aim 1: Development of miR-22 Assay for Diagnose of Metabolic Conditions in Humans

Our novel data showed that hepatic miR-22 is increased in the Western diet and alcohol-induced hepatic steatosis and its hepatic levels are positively correlated with the amount of hepatic fat content (FIG. 1). Additionally, similarly to FGF21, hepatic and blood miR-22 levels are inversely correlated. It is likely that blood miR-22 concentration can be used to predict hepatic fat and other metabolic conditions. Thus, we propose to establish a clinical assay that quantifies blood miR-22. We will establish the relationship between blood miR-22 copy number and liver pathology (the degree of steatosis, steatosis vs. steatohepatitis), BMI, serum lipid, as well as liver function, C-reactive protein, hemoglobin A1C, and Homeostasis Model Assessment score in both genders. The benchmark for success is evaluated by whether blood miR-22 can reliably predict any of those metabolic conditions making miR-22 as a treatment target.

Aim 2: Study miR-22 Inhibitors for Steatohepatitis Treatment in Mice

Our exciting preliminary data revealed the promising effect of miR-22 inhibitors in reducing hepatic fat content in mouse models. Our data also showed the effect of miR-22 mimics in inducing hepatic lymphocyte infiltration as well as hepatic fat. This aim studies the effect of miR-22 inhibitors for NASH treatment in mice. Additionally, because synthetic bile acid obeticholic acid (OCA) induces both FGF21 and miR-22 and is in a clinical trial for NASH treatment, we propose to study the combination effect of OCA and miR-22 inhibitors for NASH treatment. Positive findings of this aim will lead to use miR-22 inhibitors or a combination of miR-22 inhibitors and any medications that target FGF21-AMPK pathway, such as metformin or FGF21 itself in NASH treatment.

### Significance

Obesity has already become an epidemic disease, with nearly one-third of the global population classified as overweight or obese, and there is no doubt that the incidence of obesity-related digestive diseases will continue to rise (1,2). There is an urgent need to develop novel diagnostic and treatment means for metabolic diseases. This project develops and evaluates whether miR-22 can be used for diagnosis of metabolic diseases and treatment of non-alcoholic steatohepatitis (NASH).

Fibroblast growth factor 21 (FGF21) is a master metabolic regulator that can reverse diabetes and obesity (3-5). FGF21 regulates the metabolism of glucose, fatty acids, and ketone bodies, the energy-providing fuels for human body (6-8). Blood FGF21 is liver-derived although FGF21 can be produced by the liver, adipose tissues, and pancreas (9-11). Our data presented below revealed that hepatic and serum FGF21 levels are inversely correlated. In humans, the level of circulating FGF21 increases progressively with the advancement of hyperglycemia from prediabetes to diabetes (12). An elevated baseline FGF21, along with waist circumference and fasting plasma glucose levels, is a strong predictor of Type 2 Diabetes Mellitus (T2DM) development (12). These observations suggest that FGF21 can serve as a biomarker for T2DM risk, especially in individuals with central obesity. In addition, FGF21 signaling pathway is a therapeutic target for T2DM treatment (13,14). A clinical trial of FGF21 mimetic also generated promising results with dyslipidemia improvement (15). For patients with FGF21 resistance, pharmacologic activation of FGF21 receptor (FGFR1) and FGF21 binding protein (βKlotho) has been considered. Using an agonistic antibody specific for FGFR1 to stimulate the receptor resulted in reduced body weight and improved insulin sensitivity (16,17). Despite growing evidence for the therapeutic potential of FGF21, FGF21 resistance and its potential side effects, such as bone loss, are of concern (18). Thus, it is essential to develop novel intervention that can improve the efficacy of FGF21. The induction of FGF21 increases PGC1α, thereby activating AMPK (7). Activation of AMPK has been viewed as an important strategy for the treatment of insulin resistance. Thus, FGF21 may partially mediate the anti-diabetic effect of metformin (19,20). Importantly, activation of AMPK also reduces JAK1 signaling and has an anti-inflammatory effect (21). Together, induction of hepatic FGF21 improves metabolism, increases insulin sensitivity, and reduces inflammation.

In an effort to improve FGF21 efficacy, we uncovered that miR-22 reduces FGF21 as well as FGFR1 (data presented below). Consequently, miR-22 mimics reduce PGC1α and deactivate AMPK. Thus, miR-22 inhibitors should be able to increase FGF21 signaling, leading to improved metabolism and reduced inflammation. One aim of this proposed project is to study the effect of miR-22 inhibitors for NASH treatment. Our novel findings also revealed that hepatic miR-22 is consistently increased in fatty liver using both mouse models as well as human specimens. In addition, our data showed that the induction of hepatic miR-22 positively correlated with hepatic fat content. Moreover, just like FGF21, the hepatic and serum level of miR-22 are inversely correlated. Thus, we propose to establish a clinical test that quantifies serum miR-22 for metabolic disease diagnosis.

Being overweight and obesity predispose millions of Americans to metabolic diseases including fatty liver induced by alcohol or diet, NASH, T2DM, and liver cancer. In the U.S., minorities are at particular risk for fatty liver and tend to have a more aggressive disease course (22-25). Furthermore, there is an obvious gender difference in metabolic diseases. Population-based studies showed that NAFLD and NASH are more prevalent in men than women (23,26). The prevalence of T2DM is higher in men than women in people younger than 60 years old (27). Moreover, liver cancer is also a disease predominantly affecting men. Thus, it is crucial to develop diagnostic tools and treatment regimens that are gender-specific. For the first time, our findings revealed that the expression level of hepatic miR-22, as well as FGF21, is gender different. We aim to develop diagnostic tool that can precisely predict metabolic diseases in both genders.

### Innovation

The presented data, as well as the idea, are novel. For the first time, we showed that miR-22 is induced in the Western diet as well as alcohol-induced fatty liver. The specific effect of miR-22 in reducing hepatic FGF21, FGFR1, PGC1α, and AMPK all together has never been published before. The idea that miR-22 inhibitors may able to boost the effect of FGF21 and any drugs that target FGF21-AMPK pathway, such as metformin or obeticholic acid (explained below) are novel. We have generated preliminary data to support the idea that blood miR-22 can be a diagnostic marker for steatosis and that miR-22 inhibitors can be used to treat NASH. Moreover, we propose an innovative concept of metabolism homeostasis where it is maintained by coordinated induction of FGF21 and miR-22 that exerts opposing metabolic effect because natural chemicals found in human body including bile acids, retinoic acid, and butyrate can coordinately induce both FGF21 as well as miR-22.

### Research Strategy

Aim 1: Development of Blood miR-22 Assay for Diagnose of Metabolic Conditions in Human

### Rationale and Overall Strategy

Hepatic miR-22 is Increased in Fatty Livers in Mouse Models and Human Specimens

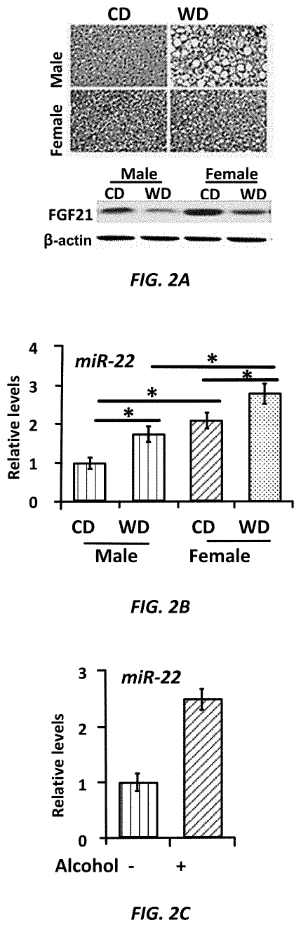

By screening the diagnostic markers for steatosis, we found the miR-22 is consistently induced in mouse and human fatty livers regardless of the etiology. After weaning, male and female C57BL/6 mice (3 weeks) were fed a control diet (CD, 5.2% fat, 12% sucrose, and 0.01% cholesterol) or Western diet (WD, 21.2% fat, 34% sucrose, and 0.2% cholesterol). The high fat and sucrose content present in the WD is like a modern diet consumed in our society (Teklad Diets). This WD increased serum cholesterol and triglyceride levels as well as fasting blood glucose levels when mice were 5 months old. In addition, WD-induced fatty liver was more severe in males than females (FIG. 2A). The protein level of hepatic FGF21 was reduced by WD. However, WD increased hepatic miR-22 in both genders. Females consistently had higher hepatic FGF21 and miR-22 than the males regardless the diet used (FIGS. 2A and 2B).

Our data revealed that hepatic miR-22 level was also increased in alcohol-induced fatty liver. We used the NIAAA model to feed mice with ethanol (28). C57BL/6 male mice were fed with a liquid diet for 5 days. Then, mice were randomly divided into pair-fed and ethanol-fed (5% ethanol) groups using the same liquid diet. Ten days after ethanol feeding, alcohol-fed mice had one dose of alcohol (5 g/kg), which is considered as binge, on day 11. All the mice were euthanized on day 12 after the initiation of ethanol feeding. Our data showed that hepatic miR-22 was induced in ethanol-induced fatty livers (FIG. 2C).

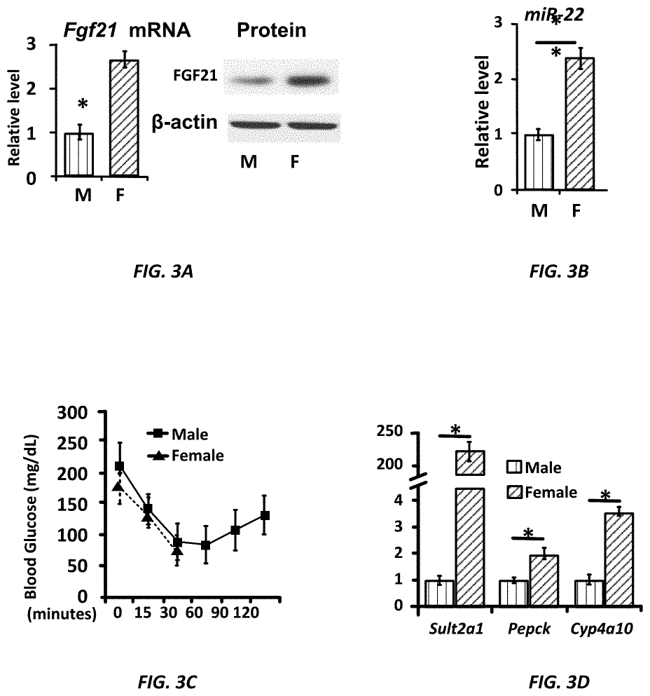

In another independent experiment, our data revealed that gender difference in metabolism is closely associated with gender difference in hepatic FGF21 and miR-22 levels. Both hepatic FGF21 and miR-22 levels were higher in female than male mice (FIGS. 3A and 3B). Consistently, age-matched female mice were highly sensitive to insulin, and female mice became hypoglycemic after 60 minutes of insulin injection and required glucose injection for rescue, whereas age-matched male mice did not have this issue (FIG. 3C). Additionally, the expressions of hepatic metabolic genes including Sult2a1 (bile salt sulfotransferase), Pepck (gluconeogenesis), and Cyp4a10 (lipid oxidation) were higher in female than male mice (FIG. 3D). Thus, coordinated induction of FGF21 or miR-22 is associated with superior metabolic condition in females. In contrast, reduced hepatic FGF21 and increased miR-22 is associated with fatty liver development.

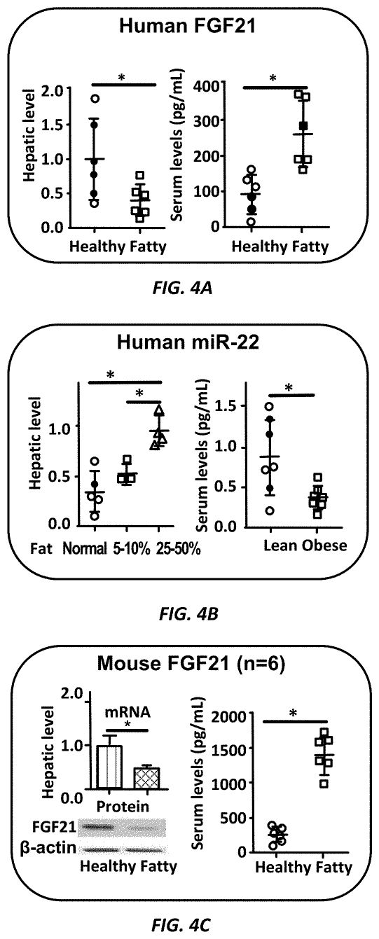

Human fatty livers also have elevated miR-22. We studied FGF21 and miR-22 levels in fatty livers obtained from bariatric surgery patients who had BMIs greater than 35. Healthy livers were obtained. Our data showed that hepatic FGF21 transcript levels were reduced in patients with hepatic steatosis (FIG. 4A). In addition, serum FGF21 mRNA levels were inversely correlated (r=−0.31) with hepatic FGF21 protein levels (FIG. 4A). Consistent with the data generated from the animal study, hepatic miR-22 levels were increased in fatty livers. In addition, the levels were positively associated with hepatic fat content evaluated by histology. Moreover, there was an inverse relationship between the hepatic and serum level of miR-22 (FIG. 4B). Together, the level of FGF21 and miR-22 is inversely correlated when patients have metabolic issues, which is consistent with the animal study.

Chemicals that Facilitate Metabolism Induce miR-22

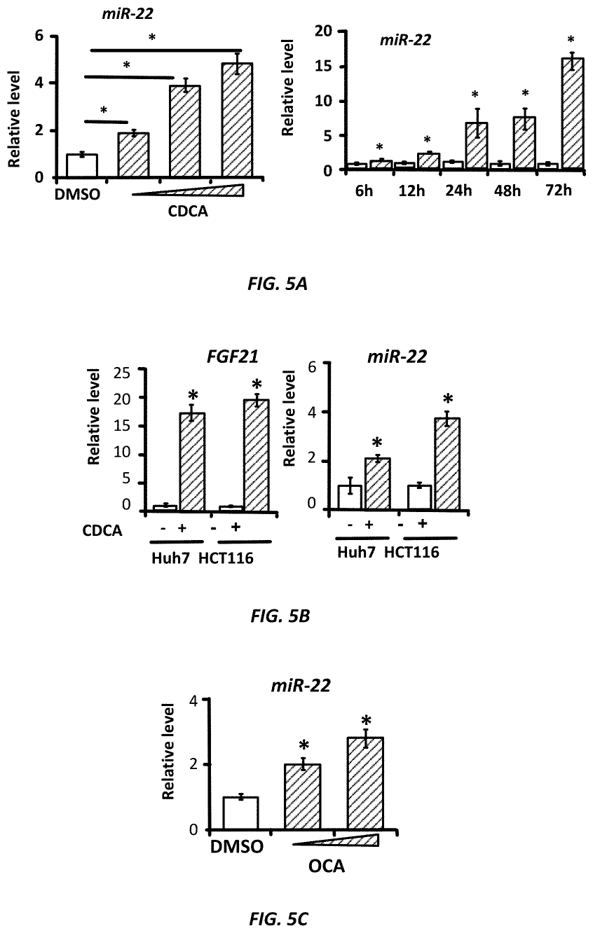

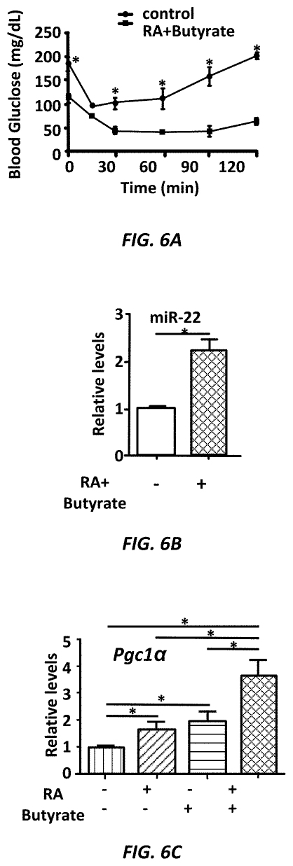

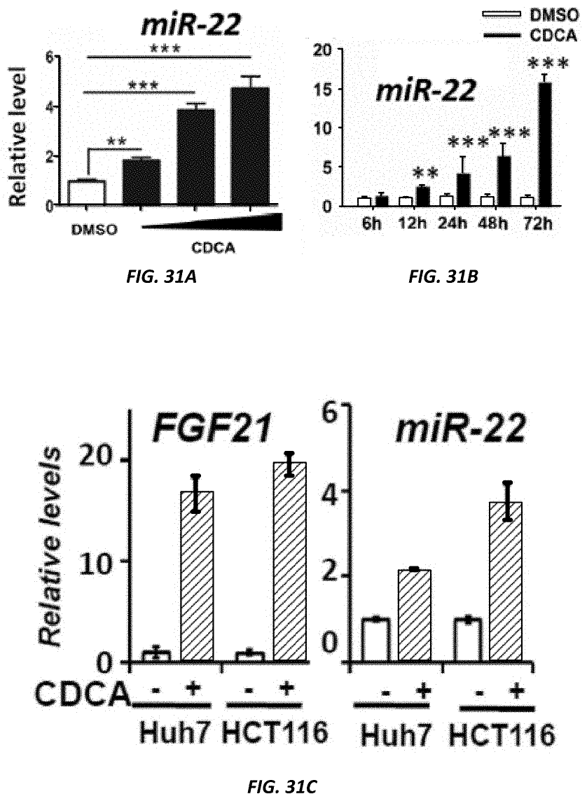

To understand the biological and pathological effects of miR-22, we explored the types of chemicals that can regulate the level of miR-22. We studied whether the chemicals that induce FGF21 can also regulate miR-22. Surprisingly, our exciting data showed that bile acids, retinoic acid, and butyrate, which have known property to induce FGF21 and facilitate metabolism, also induced miR-22 (29) (FIGS. 5 and 6). Dose response and time course studies revealed that primary bile acid, i.e., chenodeoxycholic acid (CDCA), an agonist for farnesoid x receptor (FXR, bile acid receptor), induced miR-22 (FIG. 5A). Another independent experiment showed that CDCA induced FGF21 and miR-22 levels in human liver (Huh7) as well as colon (HCT116) cells (FIGS. 5B and 31).

It is exciting that obeticholic acid (OCA, also known as INT-747) also induced miR-22 (FIG. 5C). OCA is a semi-synthetic bile acid, which has the structure 6a-ethyl-chenodeoxycholic acid. It is known that the action of OCA is to activate FXR and improve lipid and carbohydrate metabolism. Activation of FXR also improves insulin sensitivity, reduces inflammation, and has an anti-cancer effect (30-34). Currently, OCA is in the phase III trial for NASH and phase IV studies for primary biliary cirrhosis (35, 36). For the first time, our novel finding shows that OCA is a miR-22 inducer.

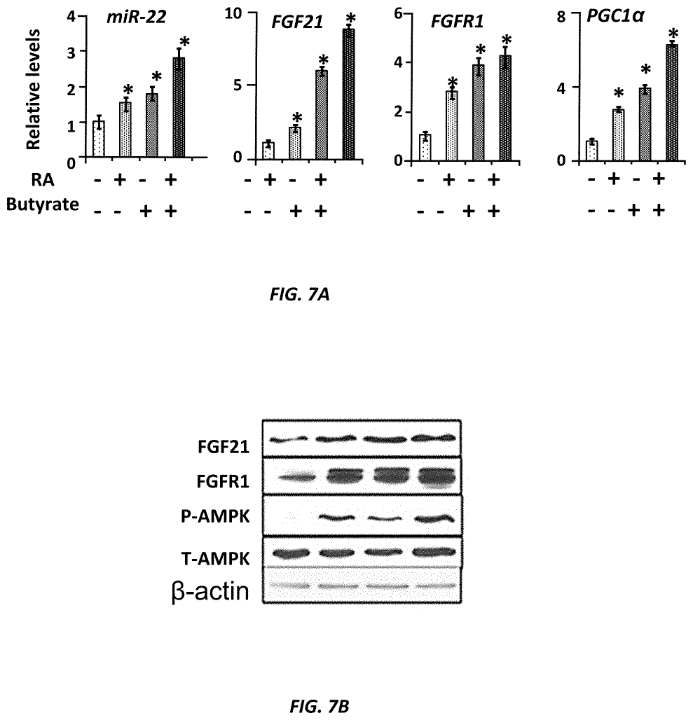

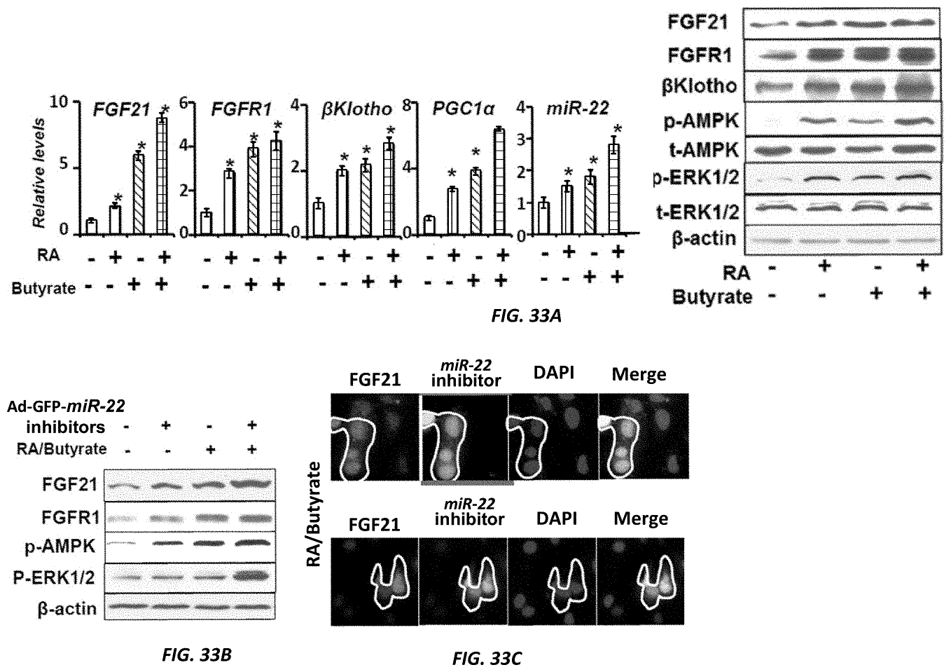

Other chemicals that can induce miR-22 are retinoic acid (RA) and butyrate. RA is naturally generated in the gut and liver by oxidation of retinol, i.e. vitamin A, and butyrate is a gut bacteria-generated short chain fatty acid (37,38). Our published data showed that RA has a profound effect in lipid metabolism (39-41). Butyrate has known effect in improving insulin sensitivity and metabolism (42-44). T2DM patients have a reduction in butyrate-producing bacterial species, and lower levels of butyrate biosynthesis (45-47). Our data generated from animal study showed that a combination of RA (15 μg/g) plus butyrate (3.2 mg/g body weight) (oral gavage, once/week for 3 weeks) substantially improved insulin sensitivity, increased hepatic miR-22, and induced hepatic Pgc1α that controls mitochondria biogenesis, a downstream effect of FGF21 (7) (FIG. 6). Consistently, cell culture experiment revealed the effect of RA and butyrate in inducing miR-22. Within 6 hours of treatment, RA and butyrate induced miR-22, FGF21, FGFR1, and PGC1α in Huh7 cells (FIGS. 7A and 33A). Consistently, their protein levels were also induced, which was accompanied by increased p-AMPK unequivocally indicating metabolic improvement (FIGS. 7B and 33B). Together, both in vivo and cell culture data revealed that coordinated induction of miR-22 and FGF21 is associated with increased metabolism in normal healthy condition.

miR-22 as a Metabolic Restrictor to Maintain Metabolism Homeostasis

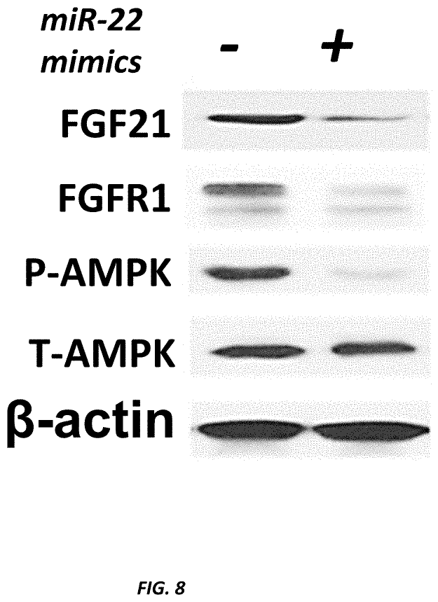

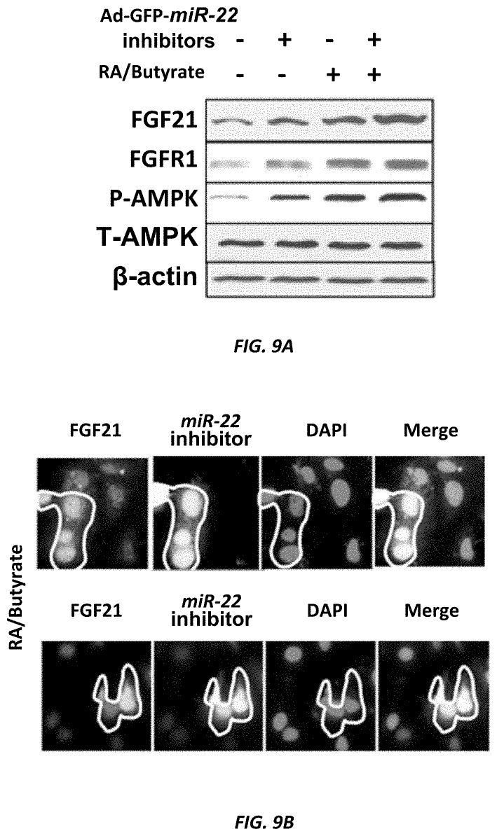

Although chemicals that facilitate metabolism induced miR-22, our data showed that miR-22 mimics reduced the protein level of FGF21, FGFR1 and p-AMPK, but had no effect on total AMPK (FIG. 8). Thus, we hypothesize that the coordinated induction of miR-22 and FGF21, as well as FGFR1, is for miR-22 to restrict consistent elevated metabolism (FIG. 1). Higher level of FGF21 requires more miR-22 to restrict its effect. Thus, females have higher FGF21 as well as miR-22 than males (FIG. 2). To prove this scenario, we used adenoviral-delivery of miR-22 inhibitors (i.e., SEQ ID NO:1) tagged with green fluorescence (adeno-miR-22 inhibitor-3P-GFP) to infect Huh7 cells. Our data showed that miR-22 inhibitors boosted the effect of RA and butyrate in inducing FGF21 and FGFR1 as and as well as AMPK activation (FIG. 9A). Moreover, immunostaining data revealed that increased FGF21 and FGFR1 levels (red in FIG. 9B) were only found in cells that had miR-22 inhibitors labeled by green fluoresce (FIG. 9B).

Together, our data support that uncoordinated expression of FGF21 and miR-22 disrupts metabolic homeostasis (FIG. 1). Overexpression of miR-22 likely led to the reduced hepatic FGF21 and FGFR1 found in fatty livers. The data generated from human specimens indicated that blood levels of miR-22 can be a diagnostic marker for steatosis or other metabolism compromised conditions. Based on the significant role of miR-22 in regulating metabolism, we propose to quantify miR-22 in human blood to establish the relationship between blood miR-22 concentration, BMI, hepatic fat content, inflammation, and Homeostasis Model Assessment (HOMA) score.

### Methodology and Analyses

Control blood samples are obtained from individuals undergoing routine colonoscopy and have normal BMI (<25) and normal colon. Associated clinical data will be obtained that includes liver pathology, BMI, age range, gender, lipid profile (total cholesterol, HDL, LDL, and triglycerides), liver function (AST, ALT, ALP, bilirubin, albumin), C-reactive protein (an inflammatory marker), and hemoglobin A1C as well as HOMA-IR. Study subjects should not receive any hypolipidemic drugs or medications for diabetic treatment that can affect metabolism. We will use blood samples from gender and age-matched people. Table 1 summarizes the specimens required for this project.

TABLE 1
Summary of required specimens.

| | Quantification of blood miR-22 |
|---|---|
| | (n = 50, 25 per gender) |
| Healthy controls | BMI <25 (50) |
| T2DM | BMI <25 with T2DM (50) |
| Obese Patients | BMI = 25-35 with (50) and without T2DM (50) |
| Obese Patients | BMI >35 with (50) and without T2DM (50) |

The following describe the quantification method:
1) Serum miRNA will be extracted by miRNeasy Serum/Plasma Kit (Qiagen). Because variability in protein and lipid content may affect RNA extraction and introduce potential inhibitors for PCR, we will include a spike-in elegans synthetic miRNAs after the initial denaturation of serum for normalization of technical variations between samples.
2) Reverse transcription will be performed using the Taq-Man MiRNA Reverse Transcription Kit and miRNA-specific stem-loop primers (Applied BioSystems).
3) Absolute quantification of miR-22 will be performed using synthetic miR-22 standards. qRT-PCR of synthetic miR-22 serial dilutions will be run in parallel with experimental samples, beginning from the reverse transcription step onwards. Plotting Ct values versus copy number of the synthetic miR-22 in a standard curve allows us to calculate miR-22 copy number based on Ct value.

All analyses will be performed for each gender separately. We will use analysis of variance (ANOVA) to compare miR-22 levels by group (BMI<25±T2DM, BMI 25-35±T2DM, BMI>35±T2DM) followed by Tukey's HSD (Honestly Significant Difference) test; if necessary, miR-22 copy number will be log transformed to achieve approximate normality and homogeneity of variance. In addition, we will compute Spearman or Pearson correlation coefficients with 95% confidence intervals to assess the relationship between miR-22 level and numeric clinical variables related to obesity, inflammation, insulin resistance, and liver function, as specified below; miR-22 levels of patients with and without liver pathology will be compared using a t-test. In addition, we will perform age-adjusted analyses using analysis of covariance (ANCOVA) to compare groups with respect to miR-22 levels, and multiple regression models to assess relationships between miR-22 and clinical variables. Statistical significance will be assessed at the 0.05 level, 2-sided. A sample size of 150 per gender provides 80% power to detect Cohen's effect size for ANOVA=0.3 (medium) with 6 groups, or a correlation coefficient of 0.25, similar to correlations between serum FGF21 and cardio-metabolic risk factors found by Zhang et al. (3).

### Expected Results and Alternative Approaches:

We anticipate that blood miR-22 concentration will be much lower in obese than lean people. Similarly, the level of blood miR-22 is expected to be lower in T2DM patients than healthy individuals. Obese patients who have T2DM may have the lowest level of blood miR-22. We will analyze the relationship between blood miR-22 copy number with liver pathology, BMI, total cholesterol, HDL, LDL, and triglycerides, as well as liver function, C-reactive protein, hemoglobin A1C, and HOMA score in each gender. The reduced blood miR-22 concentration reflects increased hepatic miR-22 and other tissues including muscle and fat. As miR-22 is ubiquitously expressed, we anticipate that increased tissue miR-22 has a systemic effect. Thus, in addition to steatosis, blood miR-22 copy number is likely to be a predictor for HOMA score and serum lipid level. This scenario is supported by our data that miR-22 affects AMPK activation. Due to the anti-inflammatory role of AMPK, we anticipate that blood miR-22 levels will be inversely correlated with C-reactive protein concentration. This expectation is also supported by the data presented below, which show tail vein injection of miR-22 mimics induced hepatic lymphocyte infiltration in mice. We expect that men and women have different normal range of blood miR-22 concentration. Thus, the data need to be analyzed based on gender to reveal a clear relationship. If necessary, we will study miR-22 levels in the livers and adipose tissues obtained from bariatric surgery patients and determine whether there is a relationship between the level of tissue miR-22 and clinical data.

Aim 2: Study miR-22 Inhibitors for Steatohepatitis Treatment in Mice

### Rationale and Overall Strategy

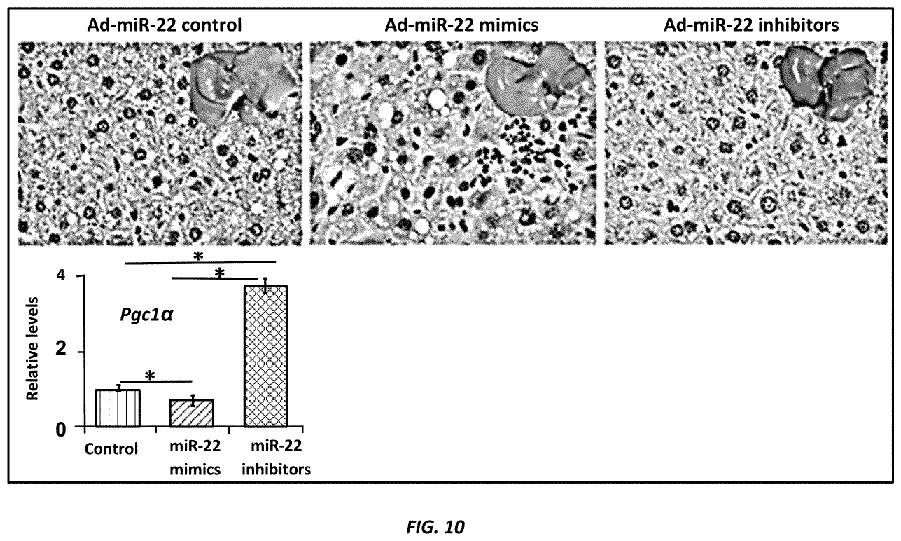

A pilot study revealed the promising effect of miR-22 inhibitors in steatohepatitis treatment. Wild type C57BL/6 mice were fed with a high fat and high sucrose (21.2% fat, 34% sucrose, and 0.2% cholesterol) Western diet (WD) since weaning (3 weeks). When mice were 2.5 months old, they received adenoviral-delivery of miR-22 mimics or miR-22 inhibitors (i.e., SEQ ID NO:1) via tail vein injection (1×10⁹ PFU/100 μl saline, 3 doses in 10 days). Gross, as well as histological data, revealed that miR-22 mimics increased the severity of steatosis and induced hepatic lymphocyte infiltration, whereas miR-22 inhibitors reduced hepatic fat content. In addition, hepatic Pgc1α mRNA level was reduced by miR-22 mimics, but increased by miR-22 inhibitors (FIG. 10). Because miR-22 mimics not only increased hepatic steatosis, but also induced hepatic lymphocyte infiltration, the data suggested that miR-22 inhibitors are likely to have a similar effect as OCA and can be used to treat NASH.

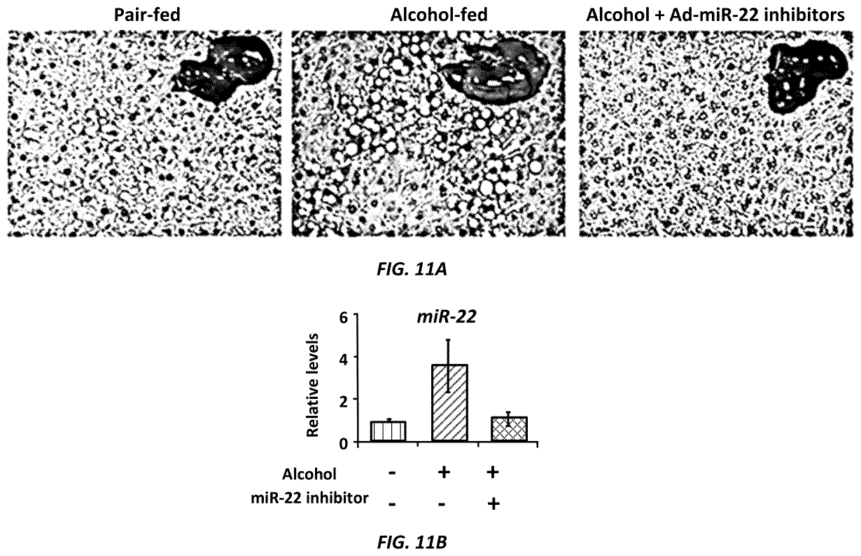

We performed another pilot experiment to test the effect of miR-22 inhibitors in reducing alcohol-induced steatosis. The same method described above for FIG. 2C was used to induce steatosis followed by miR-22 inhibitor treatment through tail vein injection. Liver histology shown in FIG. 11 revealed that miR-22 inhibitors reversed alcohol-induced fatty liver. Together, these two pilot studies provided convincing data to support the effect of miR-22 inhibitors in steatosis treatment.

Because alcohol, as well as the diet we used (high fat and sucrose), only induced steatosis without severe inflammation, we propose to use a fructose-enriched “fast food diet” to induce NASH and study the effect of miR-22 inhibitor in single or in combination with OCA in NASH treatment. This fast food diet contains fructose, saturated fats and cholesterol, providing 40% of energy as fat with 2% cholesterol (AIN-76 Western Diet, Test Diet). In addition, high-fructose corn syrup (42 g/L) will be included in the drinking water (48,49). This diet effectively induces steatosis, inflammation, and fibrosis and is used for NASH intervention study (49).

### Methodology and Analyses:

A fast food diet will be provided to male and female C57BL/6 mice after weaning. When mice are 5 months old, they will be randomly divided into 3 groups and receive control, miR-22 mimics, or miR-22 inhibitors via tail vein injection (1×10⁹ PFU/100 μl saline, twice per week).

Another animal experiment will be performed to study the combination effect of OCA and miR-22 inhibitors. Fast food-fed mice (5 months old) will be given OCA (10 mg/kg by gavage, three times a week for 2 months) with and without miR-22 inhibitors. The dose and frequency of treatment was based on the published data in rodent model (50,51). All mice in both experiments will be euthanized at 7 months of age. The experimental groups are summarized in Table 2 below.

TABLE 2
Summary of experimental groups.
Fast food-fed C57BL/6 male and female mice

| | |
|---|---|
| — | Adeno-miRNA-GFP scramble control (control) |
| — | Adeno-miR-22-3P-GFP (mimic) |
| — | Adeno-miR-22-3P inhibitor-GFP (inhibitor) |
| OCA | Adeno-miRNA-inhibitor-GFP scramble control (control) |
| OCA | Adeno-miR-22-3P inhibitor-GFP (inhibitor) |

For mouse phenotype characterization, we will record body weight, food intake, as well as liver and fat pad weight. Liver morphological analysis will be done using light microscopy, Oil Red 0 for fat content, and Masson's trichrome for fibrosis. Liver pathology will be evaluated by a liver pathologist. We will evaluate liver function and injury based on serum ALT, AST, and ALP. We will perform blood glucose tolerance test as well as insulin tolerance test once a month (3-7 months). Serum and hepatic triglyceride and cholesterol levels will be quantified.

We propose to study the expression of key metabolism genes including hepatic Pgc1α (mitochondria biogenesis), Pepck, G6Pase, Pkm (pyruvate kinase) (enzymes for gluconeogenesis or glycolysis), Srebp-1c, Fan, Cd36, and Cyp4a10 that are responsible for fatty acid synthesis, translocation, and oxidation. We will also measure hepatic miR-22 level by real-time PCR and GFP staining as well as perform Western blot to quantify hepatic FGF21, FGFR1, and p-AMPK level. Additionally, blood miR-22 will be quantified.

To monitor hepatic inflammation and fibrosis, hepatic IL-1β, IL-6, IL-10, and TNFα as well as α-actin and collagen IV mRNA levels will be quantified. Additionally, serum lipopolysaccharide, an endotoxin, level will be quantified.

### Expected Results and Alternative Approaches:

We expect that miR-22 inhibitors will reduce hepatic fat and inflammation, and therefore have an effect in NASH treatment. In contrast, miR-22 mimics are likely to aggravate the pathology accompanied by increased hepatic fat, inflammation, and fibrosis. We also expect that miR-22 inhibitors can further improve the effect of OCA in NASH treatment. Thus, a low dose OCA in combination with miR-22 inhibitors can achieve the same effect as OCA by itself (i.e., at a higher dose). This will be studied by additional dose-response experiments once the combination effect is confirmed. Using blood and hepatic miR-22 levels, we anticipate that blood miR-22 level can be a predictor for hepatic miR-22 that reflects the severity of NASH in mice. Through tail vein injection, majority (90%) miR-22 inhibitors and mimics will be in the liver. However, we will also study whether exogenous miR-22 inhibitors or mimics have an impact on body and fat weight. If a systemic effect is indicated, we can test the effect of miR-22 through intradermal or intraperitoneal injection to target other organs. We also expect that female mice will be protected from fast food-induced NASH. Thus, we may consider treating female mice with miR-22 inhibitors less frequently or use a lower dose.

### REFERENCES

1. Flegal K M, Carroll M D, Kit B K, Ogden C L: Prevalence of obesity and trends in the distribution of body mass index among U S adults, 1999-2010. Jama 2012, 307(5):491-497.
2. Ogden C L, Carroll M D, Kit B K, Flegal K M: Prevalence of obesity and trends in body mass index among U S children and adolescents, 1999-2010. Jama 2012, 307(5):483-490.
3. Zhang X, Yeung D C, Karpisek M, Stejskal D, Zhou Z G, Liu F, Wong R L, Chow W S, Tso A W, Lam K S et al: Serum FGF21 levels are increased in obesity and are independently associated with the metabolic syndrome in humans. Diabetes 2008, 57(5):1246-1253.
4. Kim K H, Lee M S: FGF21 as a mediator of adaptive responses to stress and metabolic benefits of anti-diabetic drugs. The Journal of endocrinology 2015, 226(1):R1-16.
5. Woo Y C, Xu A, Wang Y, Lam K S: Fibroblast growth factor 21 as an emerging metabolic regulator: clinical perspectives. Clinical endocrinology 2013, 78(4):489-496.
6. Potthoff M J, Inagaki T, Satapati S, Ding X, He T, Goetz R, Mohammadi M, Finck B N, Mangelsdorf D J, Kliewer S A et al: FGF21 induces PGC-1alpha and regulates carbohydrate and fatty acid metabolism during the adaptive starvation response. Proceedings of the National Academy of Sciences of the U.S. Pat. No. 2,009,106(26):10853-10858.
7. Chau M D, Gao J, Yang Q, Wu Z, Gromada J: Fibroblast growth factor 21 regulates energy metabolism by activating the AMPK-SIRT1-PGC-1alpha pathway. Proceedings of the National Academy of Sciences of the U.S. Pat. No. 2,010,107(28):12553-12558.
8. Badman M K, Pissios P, Kennedy A R, Koukos G, Flier J S, Maratos-Flier E: Hepatic fibroblast growth factor 21 is regulated by PPARalpha and is a key mediator of hepatic lipid metabolism in ketotic states. Cell metabolism 2007, 5(6):426-437.
9. Kharitonenkov A, Shiyanova T L, Koester A, Ford A M, Micanovic R, Galbreath E J, Sandusky G E, Hammond L J, Moyers J S, Owens R A et al: FGF-21 as a novel metabolic regulator. The Journal of clinical investigation 2005, 115(6):1627-1635.
10. Markan K R, Naber M C, Ameka M K, Anderegg M D, Mangelsdorf D J, Kliewer S A, Mohammadi M, Potthoff Mk Circulating FGF21 is liver derived and enhances glucose uptake during refeeding and overfeeding. Diabetes 2014, 63(12):4057-4063.
11. Liu W Y, Huang S, Shi K Q, Zhao C C, Chen L L, Braddock M, Chen Y P, Feng W K, Zheng M H: The role of fibroblast growth factor 21 in the pathogenesis of liver disease: a novel predictor and therapeutic target. Expert opinion on therapeutic targets 2014, 18(11):1305-1313.
12. Chen C, Cheung B M, Tso A W, Wang Y, Law L S, Ong K L, Wat N M, Xu A, Lam K S: High plasma level of fibroblast growth factor 21 is an Independent predictor of type 2 diabetes: a 5.4-year population-based prospective study in Chinese subjects. Diabetes care 2011, 34(9):2113-2115.
13. Suzuki M, Uehara Y, Motomura-Matsuzaka K, Oki J, Koyama Y, Kimura M, Asada M, Komi-Kuramochi A, Oka S, Imamura T: betaKlotho is required for fibroblast growth factor (FGF) 21 signaling through FGF receptor (FGFR) 1c and FGFR3c. Molecular endocrinology 2008, 22(4):1006-1014.
14. Zhao Y, Dunbar J D, Kharitonenkov A: FGF21 as a therapeutic reagent. Advances in experimental medicine and biology 2012, 728:214-228.
15. Gaich G, Chien J Y, Fu H, Glass L C, Deeg M A, Holland W L, Kharitonenkov A, Bumol T, Schilske H K, Moller D E: The effects of LY2405319, an FGF21 analog, in obese human subjects with type 2 diabetes. Cell metabolism 2013, 18(3):333-340.
16. Foltz I N, Hu S, King C, Wu X, Yang C, Wang W, Weiszmann J, Stevens J, Chen J S, Nuanmanee N et al: Treating diabetes and obesity with an FGF21-mimetic antibody activating the betaKlotho/FGFR1c receptor complex. Science translational medicine 2012, 4(162):162ra153.
17. Wu A L, Kolumam G, Stawicki S, Chen Y, Li J, Zavala-Solorio J, Phamluong K, Feng B, Li L, Marsters S et al: Amelioration of type 2 diabetes by antibody-mediated activation of fibroblast growth factor receptor 1. Science translational medicine 2011, 3(113):113ra126.
18. Wei W, Dutchak P A, Wang X, Ding X, Wang X, Bookout A L, Goetz R, Mohammadi M, Gerard R D, Dechow P C et al: Fibroblast growth factor 21 promotes bone loss by potentiating the effects of peroxisome proliferator-activated receptor gamma. Proceedings of the National Academy of Sciences of the U.S. Pat. No. 2,012,109(8):3143-3148.
19. Nygaard E B, Vienberg S G, Orskov C, Hansen H S, Andersen B: Metformin stimulates FGF21 expression in primary hepatocytes. Experimental diabetes research 2012, 2012:465282.
20. Kim K H, Jeong Y T, Kim S H, Jung H S, Park K S, Lee H Y, Lee M S: Metformin-induced inhibition of the mitochondrial respiratory chain increases FGF21 expression via ATF4 activation. Biochemical and biophysical research communications 2013, 440(1):76-81.
21. Rutherford C, Speirs C, Williams J J, Ewart M A, Mancini S J, Hawley S A, Delles C, Viollet B, Costa-Pereira A P, Baillie G S et al: Phosphorylation of Janus kinase 1 (JAK1) by AMP-activated protein kinase (AMPK) links energy sensing to anti-inflammatory signaling. Science signaling 2016, 9(453):ra109.
22. Davila J A, Morgan R O, Shaib Y, McGlynn K A, El-Serag H B: Diabetes increases the risk of hepatocellular carcinoma in the United States: a population based case control study. Gut 2005, 54(4):533-539.
23. Pan J J, Fallon M B: Gender and racial differences in nonalcoholic fatty liver disease. World journal of hepatology 2014, 6(5):274-283.
24. Kalia H S, Gaglio P J: The Prevalence and Pathobiology of Nonalcoholic Fatty Liver Disease in Patients of Different Races or Ethnicities. Clinics in liver disease 2016, 20(2):215-224.
25. Lazo M, Bilal U, Perez-Escamilla R: Epidemiology of NAFLD and Type 2 Diabetes: Health Disparities Among Persons of Hispanic Origin. Current diabetes reports 2015, 15(12):116.
26. Arun J, Clements R H, Lazenby A J, Leeth R R, Abrams G A: The prevalence of nonalcoholic steatohepatitis is greater in morbidly obese men compared to women. Obesity surgery 2006, 16(10):1351-1358.
27. Wild S, Roglic G, Green A, Sicree R, King H: Global prevalence of diabetes: estimates for the year 2000 and projections for 2030. Diabetes care 2004, 27(5):1047-1053.
28. Bertola A, Mathews S, Ki S H, Wang H, Gao B: Mouse model of chronic and binge ethanol feeding (the NIAAA model). Nature protocols 2013, 8(3):627-637.
29. Yang F, Hu Y, Liu H X, Wan Y J: MiR-22-silenced cyclin A expression in colon and liver cancer cells is regulated by bile acid receptor. The Journal of biological chemistry 2015, 290(10):6507-6515.
30. Ma K, Saha P K, Chan L, Moore D D: Farnesoid X receptor is essential for normal glucose homeostasis. The Journal of clinical investigation 2006, 116(4):1102-1109.
31. Wagner M, Zollner G, Trauner M: Nuclear bile acid receptor farnesoid X receptor meets nuclear factor-kappaB: new insights into hepatic inflammation. Hepatology 2008, 48(5):1383-1386.
32. Ratziu V, Goodman Z, Sanyal A: Current efforts and trends in the treatment of NASH. Journal of hepatology 2015, 62(1 Suppl): S65-75.
33. Giordano C, Barone I, Vircillo V, Panza S, Malivindi R, Gelsomino L, Pellegrino M, Rago V, Mauro L, Lanzino M et al: Activated FXR Inhibits Leptin Signaling and Counteracts Tumor-promoting Activities of Cancer-Associated Fibroblasts in Breast Malignancy. Scientific reports 2016, 6:21782.
34. Liu N, Zhao J, Wang J, Teng H, Fu Y, Yuan H: Farnesoid X receptor ligand CDCA suppresses human prostate cancer cells growth by inhibiting lipid metabolism via targeting sterol response element binding protein 1. American journal of translational research 2016, 8(11):5118-5124.
35. Ali A H, Carey E J, Lindor K D: Recent advances in the development of farnesoid X receptor agonists. Annals of translational medicine 2015, 3(1):5.
36. Bowlus C L: Obeticholic acid for the treatment of primary biliary cholangitis in adult patients: clinical utility and patient selection. Hepatic medicine: evidence and research 2016, 8:89-95.
37. Bushue N, Wan Y J: Retinoid pathway and cancer therapeutics. Advanced drug delivery reviews 2010, 62(13):1285-1298.
38. Louis P, Flint H J: Formation of propionate and butyrate by the human colonic microbiota. Environmental microbiology 2016.
39. Yang F, He Y, Liu H X, Tsuei J, Jiang X, Yang L, Wang Z T, Wan Y J: All-trans retinoic acid regulates hepatic bile acid homeostasis. Biochemical pharmacology 2014, 91(4):483-489.
40. He Y, Gong L, Fang Y, Zhan Q, Liu H X, Lu Y, Guo G L, Lehman-McKeeman L, Fang J, Wan Y J: The role of retinoic acid in hepatic lipid homeostasis defined by genomic binding and transcriptome profiling. BMC genomics 2013, 14:575.
41. Liu H X, Hu Y, Wan Y J: Microbiota and bile acid profiles in retinoic acid-primed mice that exhibit accelerated liver regeneration. Oncotarget 2016, 7(2):1096-1106.
42. Gao Z, Yin J, Zhang J, Ward R E, Martin R J, Lefevre M, Cefalu W T, Ye J: Butyrate improves insulin sensitivity and increases energy expenditure in mice. Diabetes 2009, 58(7):1509-1517.
43. den Besten G, van Eunen K, Groen A K, Venema K, Reijngoud D J, Bakker B M: The role of short-chain fatty acids in the interplay between diet, gut microbiota, and host energy metabolism. Journal of lipid research 2013, 54(9):2325-2340.
44. Donohoe D R, Garge N, Zhang X, Sun W, O'Connell™, Bunger M K, Bultman S J: The microbiome and butyrate regulate energy metabolism and autophagy in the mammalian colon. Cell metabolism 2011, 13(5):517-526.
45. Karlsson F H, Tremaroli V, Nookaew I, Bergstrom G, Behre C J, Fagerberg B, Nielsen J, Backhed F: Gut metagenome in European women with normal, impaired and diabetic glucose control. Nature 2013, 498(7452):99-103.
46. Qin J, Li Y, Cai Z, Li S, Zhu J, Zhang F, Liang S, Zhang W, Guan Y, Shen D et al: A metagenome-wide association study of gut microbiota in type 2 diabetes. Nature 2012, 490(7418):55-60.
47. Benedict C, Vogel H, Jonas W, Woting A, Blaut M, Schurmann A, Cedernaes J: Gut microbiota and glucometabolic alterations in response to recurrent partial sleep deprivation in normal-weight young individuals. Molecular metabolism 2016, 5(12):1175-1186.
48. Charlton M, Krishnan A, Viker K, Sanderson S, Cazanave S, McConico A, Masuoko H, Gores G: Fast food diet mouse: novel small animal model of NASH with ballooning, progressive fibrosis, and high physiological fidelity to the human condition. American journal of physiology Gastrointestinal and liver physiology 2011, 301(5):G825-834.
49. Bettaieb A, Jiang J X, Sasaki Y, Chao T I, Kiss Z, Chen X, Tian J, Katsuyama M, Yabe-Nishimura C, Xi Y et al: Hepatocyte Nicotinamide Adenine Dinucleotide Phosphate Reduced Oxidase 4 Regulates Stress Signaling, Fibrosis, and Insulin Sensitivity During Development of Steatohepatitis in Mice. Gastroenterology 2015, 149(2):468-480 e410.
50. Vavassori P, Mencarelli A, Renga B, Distrutti E, Fiorucci S: The bile acid receptor FXR is a modulator of intestinal innate immunity. Journal of immunology 2009, 183(10):6251-6261.
51. Fiorucci S, Antonelli E, Rizzo G, Renga B, Mencarelli A, Riccardi L, Orlandi S, Pellicciari R, Morelli A: The nuclear receptor SHP mediates inhibition of hepatic stellate cells by FXR and protects against liver fibrosis. Gastroenterology 2004, 127(5):1497-1512.

### Example 2. Epigenetic Approach for Metabolic Disease Treatment

The project described in this example studies the effect of microRNA-22 (miR-22) in diet-induced metabolic heath issues, including fatty liver and insulin resistance.

### Project Abstract

Fibroblast growth factor 21 (FGF21) and Sirtuin 1 (SIRT1) have a remarkable ability to reverse diabetes and obesity. Given their effect in AMPK activation, it is important to understand their regulatory mechanism. The action of FGF21 requires the presence of its receptor FGFR1. Our novel data revealed chemicals that have beneficial metabolic effects in improving insulin sensitivity and treating steatohepatitis simultaneously increase FGF21, FGFR1, and SIRT1, which leads to AMPK and ERK1/2 activation. However, those chemicals also concomitantly increased miR-22, which reduced FGF21, FGFR1, and SIRT1. Consistent with the undesirable metabolic effect of miR-22, increased miR-22 level was found in human fatty livers as well as diet and alcohol-induced fatty livers in mice. Additionally, exogenous miR-22 exacerbated diet-induced steatosis in mice revealing its metabolic inhibitory effect in vivo. In contrast, miR-22 inhibitors prevented diet and alcohol-induced steatosis. Based on these exciting data, we hypothesize that reducing miR-22 in a metabolically compromised condition treats non-alcoholic steatohepatitis (NASH) and improves insulin sensitivity. We also propose that inhibiting miR-22 expression enhances the therapeutic effects of chemicals that target AMPK signaling. Together, this project examines the effect and regulatory mechanism of miR-22 in metabolic disease development and treatment. Two aims are proposed to reach those specific goals.

Aim 1 studies the effect of miR-22 in metabolic disease treatment using diet-induced animal models. We will analyze the metabolic beneficial effect of miR-22 inhibitors alone or in combination with FGF21, INT-747, a bile acid receptor FXR (farnesoid x receptor) agonist, and butyrate in NASH treatment and improving insulin sensitivity. The role of miR-22 in metabolic disease development will be analyzed as well by introducing exogenous miR-22 to the liver.

Aim 2 studies the mechanism by which miR-22 is regulated that in turn affects metabolism. Given the opposing effects of miR-22 and FGF21 in activating AMPK and ERK1/2, it is fascinating that their inductions are coordinated in response to active metabolism. We will study the mechanisms for this orchestrated regulatory process. We propose to test a hypothesis that FXR and acetylation are the common factors that concomitantly regulate the expression of miR-22 and FGF21 signaling. Additionally, the differential regulatory mechanism for miR-22 induction in metabolically active and compromised conditions will be studied in mice used in Aim 1. The downstream effect of miR-22 in regulating acetylation as well as metabolism will be analyzed. The generated mechanistic information will lead to additional innovative approaches by targeting specific deacetylases to treat metabolic diseases. The proposed animal trials will lead to immediate clinical trials of using combined miR-22 inhibitors and AMPK activators such as INT-747, butyrate, or metformin.

### Specific Aims

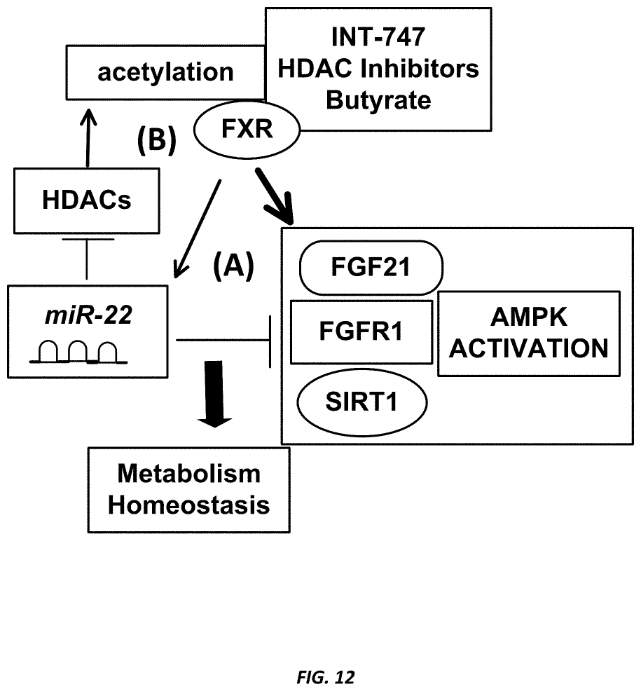

FGF21 and SIRT1 are master metabolic regulators and treatment targets for steatohepatitis and type 2 diabetes mellitus (T2DM). The action of FGF21 requires the presence of its receptor, FGFR1. Our novel data showed that bile acid receptor FXR (farnesoid x receptor) agonist and butyrate, which can improve insulin sensitivity and treat steatohepatitis, increased FGF21, FGFR1, and SIRT1 simultaneously, leading to AMPK and ERK1/2 activation. However, those chemicals also increased miR-22, which reduced FGF21, FGFR1, and SIRT1. It is likely that the metabolic inhibitory effect of miR-22 is to restrict AMPK and ERK1/2 activation avoiding persistent metabolism-driven over growth thereby maintaining homeostasis (FIG. 12A). Consistent with the undesirable metabolic effect of miR-22, increased miR-22 was found in human fatty livers as well as diet and alcohol-induced fatty livers in mice. Additionally, exogenous miR-22 exacerbated diet-induced steatosis in mice revealing its detrimental metabolic effect in vivo. In contrast, miR-22 inhibitors prevented diet and alcohol-induced steatosis in mice. Based on these exciting findings, we hypothesize that reducing miR-22 in a metabolic compromised condition treats non-alcoholic steatohepatitis (NASH) and improves insulin sensitivity. We also hypothesize that reducing miR-22 enhances the therapeutic efficacy of chemicals that target AMPK. Thus, this project studies the effect (Aim 1) and regulatory mechanism (Aim 2) of miR-22 in metabolic disease development and treatment.

Aim 1: Study the Effect of miR-22 in Metabolic Disease Development and miR-22 Inhibitors in Metabolic Disease Treatment.

Using newly generated, hepatocyte-specific FGF21 KO mice and adenoviral-delivered FGF21, our data revealed the significance of hepatic FGF21 in regulating glucose homeostasis and liver fat deposition. We propose using an epigenetic approach to increase hepatic FGF21, FGFR1, and SIRT1 leading to AMPK activation. This will be done using adenoviral delivery of a miR-22 inhibitor (e.g., SEQ ID NO:1). We will also deliver exogenous miR-22 to diet-induced obese animal models to facilitate the development of NASH and insulin resistance to understand the role of miR-22 in metabolic disease development. To study the effect of miR-22 inhibitor in enhancing the drugs used to treat NASH and T2DM, the combined effect of miR-22 inhibitors plus adenoviral-delivered FGF21, INT-747, and butyrate will be tested in diet-induced obese mice. INT-747 is a synthetic FXR agonist that has promising effects and is in clinical trials for NASH and T2DM treatment. Our novel data showed that INT-747 induced FGF21 and FGFR1 as well as their silencer, miR-22. Thus, blocking INT-747-induced miR-22 is expected to increase the efficacy of INT-747. Butyrate is naturally produced in the gut that has histone deacetylase (HDAC) inhibitory property. Like INT-747, our data showed that butyrate induced FGF21, FGFR1, and their inhibitor miR-22. Additionally, the effects of butyrate in NASH treatment and improving insulin sensitivity have been revealed by others and us. Therefore, we anticipate that inhibiting butyrate-increased miR-22 expression can improve the metabolic effects of butyrate. New clinical trials using combined miR-22 inhibitor and INT-747, butyrate, or other AMPK activators such as metformin can be implemented when the anticipated results are validated by the proposed animal experiments.

Aim 2: Study the Mechanism by which miR-22 is Regulated that in Turn Affects Metabolism.

Given the opposing effects of miR-22 and FGF21 in activating AMPK, it is fascinating that their inductions are coordinated in response to INT-747 and butyrate treatment. We will study the mechanisms for this orchestrated regulatory process in response to metabolic activators. We propose to test a hypothesis that FXR and histone acetylation are the common factors that concomitantly regulate the expression of miR-22 and FGF21 signaling. This hypothesis is based on the findings that FXR agonists and chemicals that have HDAC inhibitory property could simultaneously induce miR-22, FGF21, and FGFR1. Additionally, our novel data showed that miR-22 not only reduced deacetylase SIRT1, but also HDAC1, HDAC4, and potentially other deacetylases. Thus, acetylation may be both a downstream effect of miR-22 as well as upstream regulatory mechanisms for FXR transcriptional activation and miR-22 expression, forming self-regulatory loops (FIG. 12B). Disruption of those self-regulatory loops may lead to miR-22 induction, but FXR inactivation, thus metabolic disease development. The differential regulatory mechanism for miR-22 induction in metabolically active and compromised conditions will be studied in mice used in Aim 1 and cell culture models. Methods used include uncovering specific HDACs silenced by miR-22 followed by target validation. We will analyze the recruitment of HDACs in the regulatory region of miR-22 and the FXR gene. Additionally, the mechanism by which miR-22 reduces FGF21 and FGFR1 will also be studied as well. The generated mechanistic information will lead to additional innovative approaches by targeting specific deacetylases to treat metabolic diseases.

### Significance

### The Significance of FGF21 and SIRT1

The principal energy-providing fuels of the human body are glucose, fatty acids, and ketone bodies, and FGF21 regulates each associated pathway. Administration of FGF21 to rodents and rhesus monkeys in diet- or genetically-induced obesity as well as diabetes models causes anti-hyperglycemic and triglyceride-lowering effects and reduction of body weight (1-5). Mechanistically, FGF21 activates hepatic PGC1α (PPAR-activated receptor-γ coactivator-1α), a transcriptional co-activator required for fatty acid oxidation and gluconeogenic pathways, and activates AMPK to enhance insulin sensitivity (6). AMPK activation has a key role in the therapeutic benefits of metformin, and is a treatment target of T2DM and metabolic syndrome (7-9). Additionally, FGF21 inhibits fatty acid synthesis, stimulates β-oxidation by increasing CPT1α and CYP4A, and promotes glucose uptake by inducing GLUT-1 (10-12). Moreover, FGF21 activates the ERK1/2 survival pathway. Our published data reveal that FGF21 repairs liver injury and facilitates regeneration in vivo (13,14). Active metabolism induced by FGF21 is also implicated in longevity evidenced by the transgenic FGF21 mice have an extended lifespan (15). Moreover, compromised metabolism due to aging promotes diabetes and obesity (16). Together, AMPK and ERK1/2 activation is used to monitor the metabolic and proliferative downstream effects of FGF21 linking metabolism and growth (17,18).

SIRT1 is a NAD⁺-dependent deacetylase. Like the effect of FGF21, SIRT1 has a key role in regulating cellular processes that control metabolism, autophagy, survival (19-23), and it also plays a role in longevity (24). SIRT1 activation results in nutrient utilization and enhanced mitochondrial oxidative function to regulate energy balance. Together, both AMPK and SIRT1 act in concert with the master regulator of mitochondrial biogenesis (i.e., PGC1α) to regulate energy homeostasis (17,25). Moreover, SIRT1 and FGF21 cross talk (10). Together, an understanding of FGF21 and SIRT1 signaling has a significant impact on the prevention and treatment of metabolic disorders.

### Regulation of FGF21 and SIRT1

Nuclear receptors such as FXR and PPARα regulate the expression of FGF21 (26,27). Our novel data demonstrated that butyrate generated by Gram-positive bacterial fermentation of fibers can simultaneously induce FGF21, FGF21 receptor FGFR1, and FGF21 binding protein βKlotho, which together constitute FGF21 signaling. Because butyrate has histone deacetylase (HDAC) inhibitory property, the ability of butyrate in increasing FGF21 signaling suggests the significance of epigenetic regulatory mechanism, which is the focus of this application. Regarding SIRT1, epigenetic mechanisms in regulating the expression of SIRT1 have been comprehensively reviewed (28).

FGF21 is expressed in the liver, pancreas, adipose tissues, brain, and thyroid (29-33). Our preliminary data presented below showed the significance of hepatic FGF21 in regulating blood glucose and hepatic lipid metabolism. In addition, our exciting data showed that activation of FXR, which is abundantly found in the liver, also simultaneously increases FGF21, FGFR1, and βKlotho leading to AMPK and ERK1/2 activation. In contrast, FXR KO mice develop steatohepatitis and liver cancer spontaneously (34-39). Additionally, intestinal FXR KO mice are protected from diet-induced obesity (40,41). Based on the importance of liver FGF21 and FXR in regulating glucose and lipid metabolism, this project uses epigenetic approaches to understand the expression of hepatic FGF21 regulated by FXR.

The Significance of miR-22 in Regulating FGF21 and SIRT1 Signaling

miR-22 is highly conserved across many vertebrate species, including chimp, mouse, rat, dog, and horse, suggesting it has functional importance. The cancer protection effect of miR-22 has been demonstrated (42,43). We have also uncovered for the first time that the level of miR-22 can be induced by natural chemicals such as bile acids (BAs) that activates FXR and short chain fatty acids (SCFAs) that have HDAC inhibitory properties (43) (preliminary data described below). Those chemicals have known cancer protective and beneficial metabolic effects. However, our novel data also revealed that miR-22 reduces the protein level of FGF21, FGFR1, SIRT1 leading to AMPK and ERK1/2 deactivation thereby restricting metabolism-driven growth. Thus, the concomitant induction of FGF21 and SIRT1 as well as their silencer miR-22 by those metabolism facilitators can be a mechanism to maintain metabolic homeostasis avoiding persistent AMPK and ERK1/2 activation. This negative regulatory pathway can be very important because SIRT1 overexpression is implicated in liver carcinogenesis and found in human liver cancer (44-47).

The metabolic inhibitory effect of miR-22 is also revealed in vivo; our preliminary data revealed that miR-22 overexpression induces fatty livers and miR-22 inhibitors prevent diet and alcohol-induced fatty livers. Moreover, the level of miR-22 is positively associated with hepatic fat content and negatively correlated with the level of PGC1α in human liver biopsy specimens shown in presented data below. These exciting findings clearly indicate that coordinated induction of FGF21 and SIRT1 as well as miR-22 maintains metabolic homeostasis. In contrast, an uncoordinated expression pattern with elevated miR-22, but lack of FGF21 and SIRT1 induction, leads to metabolic compromise. Due to the effects of miR-22 in deactivation of AMPK signaling, we propose to study the effect of miR-22 inhibitors alone or in combination with FGF21, FXR agonist, and butyrate in improving insulin sensitivity and NASH treatment.

The Significant Role of miR-22 in Regulating Acetylation

Our novel data revealed that chemicals that have HDAC inhibitory properties induced miR-22, which reduced protein deacetylases including HDAC1, HDAC4, and SIRT1. Additionally, based on sequence alignment data, miR-22 may inhibit many other HDACs. Because butyrate simultaneously induces miR-22, FXR, FGF21, FGFR1, and βKlotho, it is very likely that miR-22-silenced HDACs may orchestrate this concomitant regulation. This novel concept will be studied in the proposed project. Identification of the specific deacetylases targeted by miR-22 that can regulate FXR itself as well as FGF21 signaling has a huge therapeutic impact. Such information will lead to additional novel treatment strategies for metabolic disease treatment by using specific HDAC inhibitors. Thus, another goal of this project is to understand the underlying mechanism for miR-22 expression as well as miR-22 downstream effects. Together, the proposed experiments will define the clinical relevance of miR-22 in metabolic treatment and establish novel regulatory mechanisms.

### Innovation

The proposed studies are innovative in many aspects. This is the first time that the simultaneous induction of all three components of FGF21 signaling (i.e., FGF21 itself, its receptor FGFR1, and its binding protein βKlotho) is demonstrated by a FXR agonist and butyrate. Such orchestrated induction suggests the importance of this previously unstudied regulatory mechanism controlled by FXR and acetylation. Moreover, miR-22, which deactivates AMPK by simultaneously reducing FGF21, FGFR1, and SIRT1, was uncovered. Animal trials are proposed to examine the effectiveness of miR-22 inhibitors in metabolic disease treatment as well as improving the efficacy of drugs that are used to treat T2DM and NASH. Moreover, the exogenous miR-22 will be studied to understand its role in metabolic disease development. Since the tested drugs have been used in humans to treat disease or as food supplements, positive outcomes will lead to new clinical trials.

The proposed project tests a novel mechanism that miR-22 reduces FGF21, FGFR1, and SIRT1 functions as a negative metabolic regulator. Ironically, miR-22 through its HDAC inhibitory property may increase the transcriptional activity of FXR, which is an upstream regulator for FGF21 signaling. Those conflicting roles elucidate that metabolic homeostasis is controlled at both transcriptional and post-transcriptional levels. This scenario is also supported by our novel data that showed butyrate increased the expression level of FXR, FGF21, FGFR1, βKlotho, and miR-22 suggesting epigenetic regulation of all those key metabolic regulators. By dissecting those regulatory pathways, we anticipate to uncover novel underlying mechanisms to have intact metabolism. The generated data will also identify specific HDACs that participate in the regulatory processes leading to other innovative treatment strategies.

Regarding the approaches, we propose to study the most promising drug, INT-747. Furthermore, we will use both miR-22 mimics and miR-22 inhibitors, whose roles have been validated in cell lines, mouse models, and human steatosis, to firmly establish the role of miR-22 and miR-22 inhibitors in steatohepatitis development as well as treatment, respectively. Moreover, we anticipate that forced expression of miR-22 in Western diet-fed mice can be a novel animal model to study NASH evidenced by data presented below. Furthermore, our novel data revealed that the expression level of hepatic miR-22 and FGF21 are sex different. Moreover, our published data showed that sex-difference in the development of steatosis is FXR-dependent (48). The proposed approaches provide a unique opportunity to understand whether sex difference in FXR and miR-22-regulated signaling can account for the sex difference in susceptibility in metabolic disease development. Together, we are confident that the proposed approaches will generate robust and unbiased results, which aligns with the NIH mission to encompass genders and species.

### Research Strategy

Aim 1: Study the Effect of miR-22 in Metabolic Disease Development and miR-22 Inhibitors in Metabolic Disease Treatment.

### Rationale, Preliminary Data, and Overall Strategy

### Hepatic FGF21 Affects Blood Glucose Level, Insulin Sensitivity, and the Development of NASH

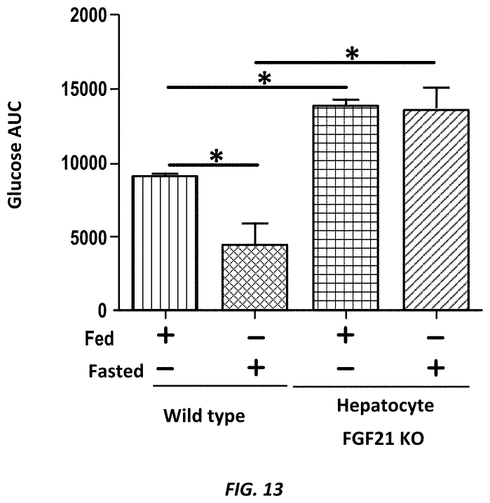

The significance of FGF21 has been revealed using whole body FGF21 KO mouse models (6). Whole body FGF21 KO mice are susceptible to metabolic disorders and NASH induced by streptozotocin or a high fat diet as revealed by a recent publication (49). FGF21 is expressed in the metabolically active tissues such as liver, muscle, adipose tissue, brain, and thyroid (29-33). To understand the significance of hepatic FGF21, we have generated novel liver-specific FGF21 KO mouse models by breeding mice harboring the Fgf21^{loxP} allele (Jackson Laboratory) (6) with mice that express albumin promoter-driven Cre recombinase (Jackson Laboratory) (50). Our initial observation showed that hepatocyte-specific FGF21 KO mice had elevated fasting blood glucose level and were insulin resistant in both fasted and fed states (FIG. 13). In addition, fasting did not increase their insulin sensitivity like what occurred in wild type mice (FIG. 13).

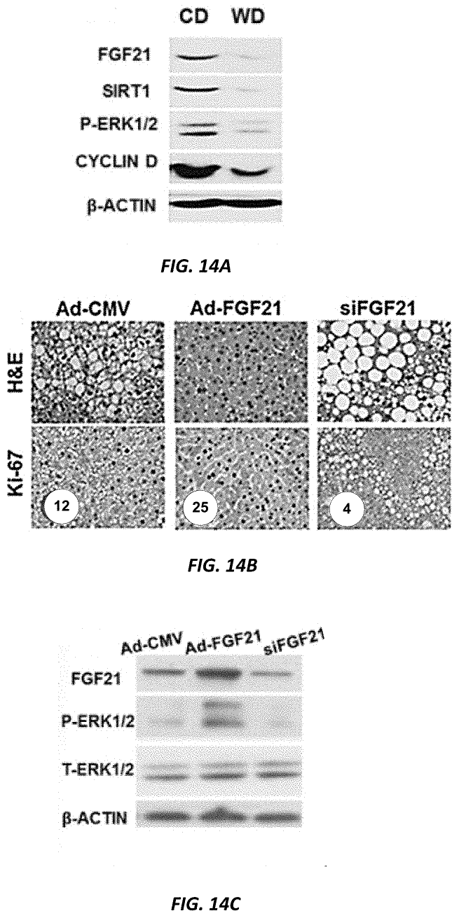

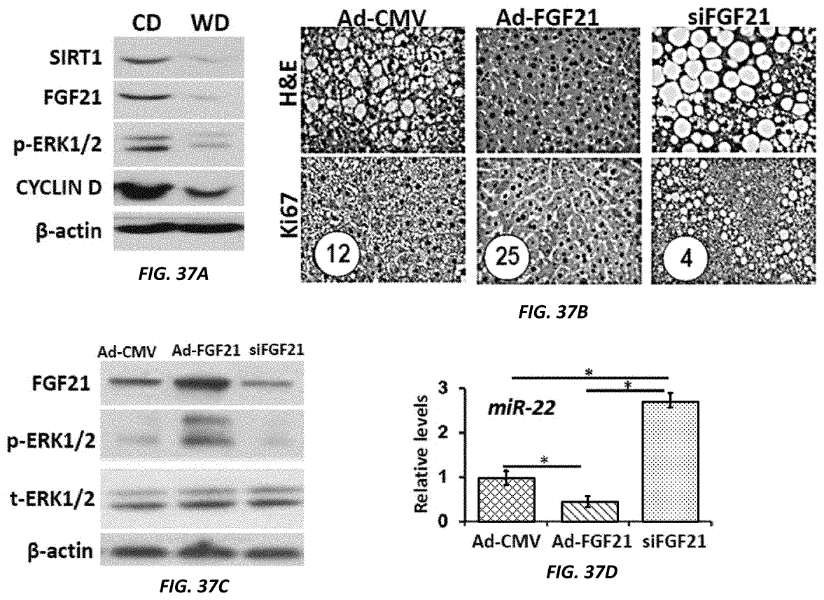

Besides AMPK activation, another important downstream effect of FGF21 is to activate ERK1/2 to support metabolism-driven growth (51). This is also supported by our published data showed that forced expression of FGF21 rescues liver regeneration impairment caused by dysregulated bile acid synthesis (14). Consistently, using 2/3 partial hepatectomy animal models, our data showed that Western diet (WD)-fed mouse livers had reduced liver regeneration capability that was accompanied by reduced FGF21 and SIRT1, deactivated ERK1/2 as well as decreased CYCLIN D (FIGS. 14A and 37A). When recombinant adenoviruses are introduced directly into blood circulation, transgene expression is almost completely restricted to the liver. Using this approach, we evaluated the significance of FGF21 in the liver. Adenoviral-delivery of FGF21 or FGF21 siRNA was used to overexpress or knock down hepatic FGF21 via tail vein injection. Our data showed that hepatic FGF21 overexpression was effective in treating WD-induced fatty liver and restored liver regeneration capability as revealed by the number of Ki-67 positive cells (FIGS. 14B and 37B). In contrast, reduced hepatic FGF21 exacerbated the severity of steatosis, reduced the number of Ki-67 positive cells, and deceased phospho-ERK1/2 (P-ERK1/2) without changing the level of total ERK1/2 (T-ERK1/2) (FIGS. 14C and 37C). Together, these findings emphasize the significance of hepatic FGF21 in regulating glucose level, insulin sensitivity, as well as hepatic metabolism and regeneration.

Hepatic miR-22 Reduces FGF21, FGFR1, and SIRT1

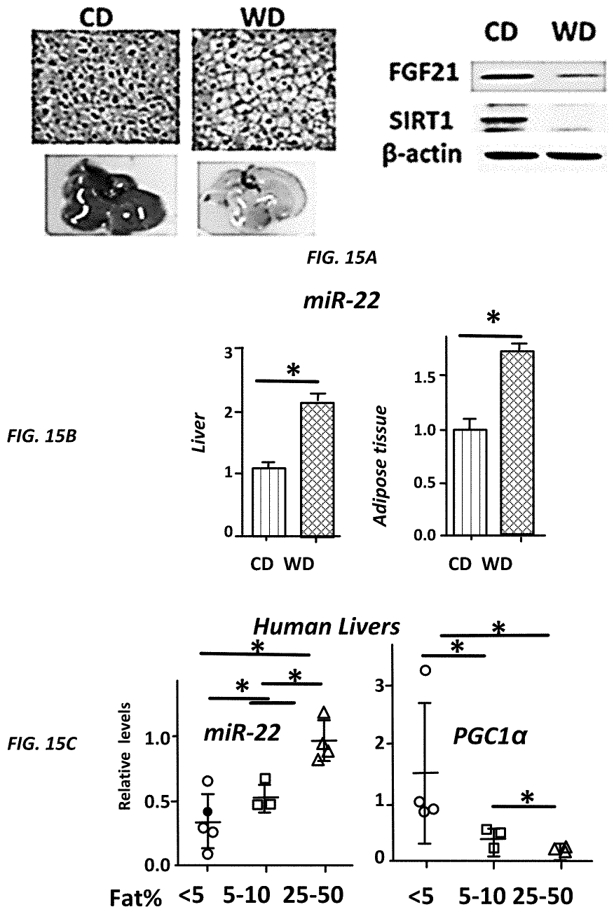

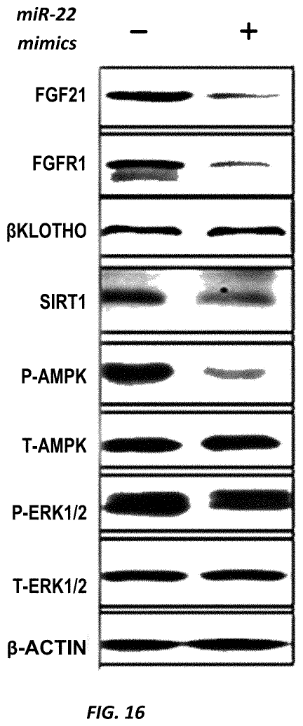

Our novel data revealed an inversed expression pattern between miR-22 and liver metabolic status. FIG. 15A shows that WD-fed mice (5-months old) had fatty livers with reduced hepatic FGF21 and SIRT1. However, miR-22 was induced in the livers and adipose tissues of WD-fed mice (FIG. 15B). Similarly, using human liver biopsy specimens, miR-22 levels were positively associated with hepatic fat content and inversely correlated with the mRNA levels of PGC-1α, a key regulator of mitochondrial biogenesis (FIG. 15C). Such positively correlated relationship between the level of hepatic miR-22 and steatosis severity suggests an undesirable effect of hepatic miR-22 on metabolism. To study the downstream effects of miR-22, miR-22 mimics (ID: MIMAT0000077, AAGCUGCCAGUUGAAGAACUGU (SEQ ID NO:2), Applied Biological Materials Inc.) were used to transfect human liver Huh7 cells. Western blot data showed that overexpression of miR-22 reduced the protein level of FGF21, FGFR1, and SIRT1 leading to AMPK and ERK1/2 deactivation (reduction in P-AMPK and P-ERK1/2) without changing the level of total AMPK and total ERK1/2 (FIG. 16). However, miR-22 did not affect the expression of βKlotho, a FGF21 binding protein (FIG. 16). Based on the ability of miR-22 to silence both FGF21 and its receptor, these findings strongly suggest the significance of hepatic miR-22 in regulating metabolism, which is one of the central focuses of this application.

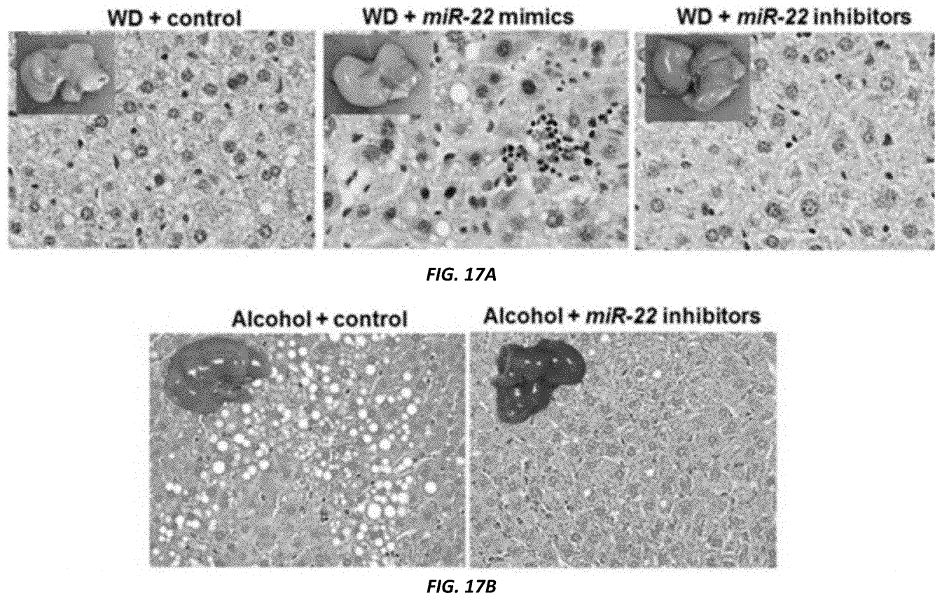

To further analyze the role miR-22 in regulating hepatic metabolism, we have done pilot studies to test the effect of miR-22 in non-alcoholic and alcoholic steatohepatitis. Steatosis was induced by feeding mice with a WD that has high fat and high sucrose content since weaning (3-weeks old). After 2 months of differential diet feeding, mild steatosis was found in WD-fed mice. Then, those WD-fed mice received adenoviral-control, adenoviral-delivered miR-22 mimics, or miR-22 inhibitors (i.e., SEQ ID NO:1) via tail vein injection (1×10⁹ pfu/200 μl, 3 doses, weekly) while continuing a WD. Histological data revealed that miR-22 mimics increased hepatic fat content and lymphocyte infiltration (FIG. 17A, middle). In contrast, miR-22 inhibitor-treated mice had normal livers grossly and histologically (FIG. 17A, right). These exciting findings indicate that forced miR-22 expression in WD-fed mice potentially can be a novel NASH model. Furthermore, FIG. 37D shows that hepatic miR-22 levels were positively associated with the severity of steatosis. These data indicate the usefulness miR-22 inhibitors in treating steatosis.

Additionally, alcohol was used to induce fatty liver. Mice were on Liber DeCarli liquid diet supplemented with 5% alcohol for 3 weeks followed by delivery of adenoviral-control and adenoviral-miR-22 inhibitors using the same methods described above. During the interventions, mice continued having alcohol-supplemented liquid diet. Similarly, miR-22 inhibitors effectively eliminated alcohol-induced fat deposition (FIG. 17B). Based on these promising data, the proposed experiments study the effect of miR-22 inhibitors as well as miR-22 inhibitors in combination with AMPK activators that include FGF21, semi-synthetic bile acid INT-747, and butyrate. Justification and preliminary data are provided below.

The Effect of INT-747 in Improving Metabolism and Ironically Inducing Metabolic Silencer miR-22

FGF21 resistance is a clinical problem and the underlying mechanism is unknown (52). It is important to improve FGF21 efficacy to avoid using increasing amount of FGF21. Thus, we propose to study the effect of miR-22 inhibitors through prohibiting the reduction of exogenous and endogenous FGF21 signaling thereby enhancing AMPK activation.

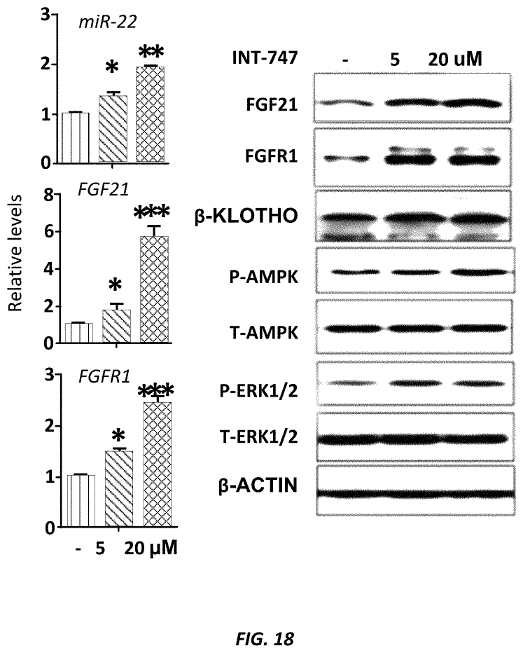

Bile acids (BAs) are not only just digestive surfactants, they regulate inflammation as well as lipid and sugar metabolism (53-57). This paradigm shift was spurred by identification of BA receptors FXR. As mentioned above that FXR KO mice develop spontaneous steatohepatitis and liver carcinogenesis (34-39); whereas FXR-selective agonist INT-747 also known as obeticholic acid (OCA) is in Phase 3 clinical trials for NASH. It has been shown that INT-747-activated FXR can increase cholesterol absorption (58). INT-747 is also a FDA-approved drug to treat biliary cholangitis (59-61). Ironically, the endogenous ligand of FXR, i.e., chenodeoxycholic acid (CDCA) as well as INT-747 induced miR-22, which is a metabolic inhibitor (43) (FIG. 18). Within 6 hours, INT-747 treatment markedly increased the mRNA and protein level of FGF21 and FGFR1 in Huh7 cells. Moreover, INT-747 also modestly increased βKlotho protein level (FIG. 18). Furthermore, INT-747 activated AMPK and ERK1/2 revealing its beneficial effect in metabolism as well as tissue repair (FIG. 18). Together, the simultaneous induction of all three components of FGF21 signaling by INT-747 indicates the significance of FGF21 pathway in mediating the metabolic beneficial effect of INT-747. Based on these promising data, we anticipate reducing miR-22 can increase the efficacy of INT-747 in NASH and T2DM treatment.

The Effect of Butyrate in Improving Metabolism and Ironically Inducing Metabolic Silencer miR-22

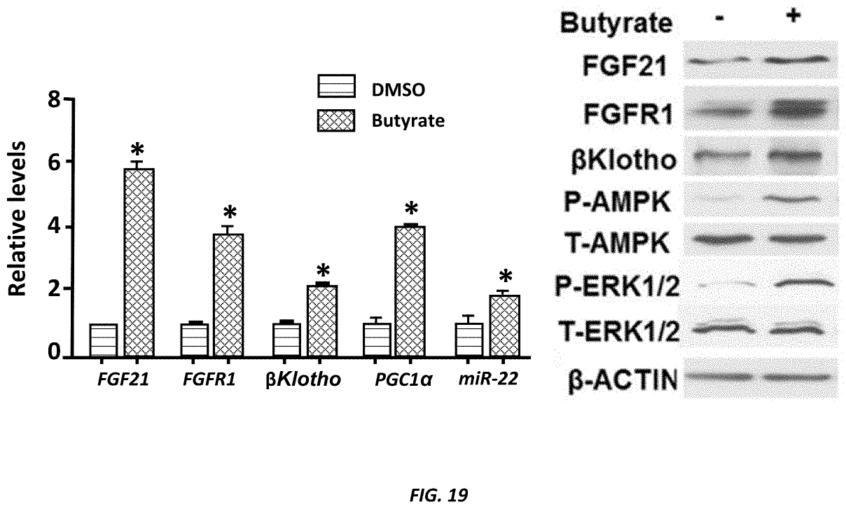

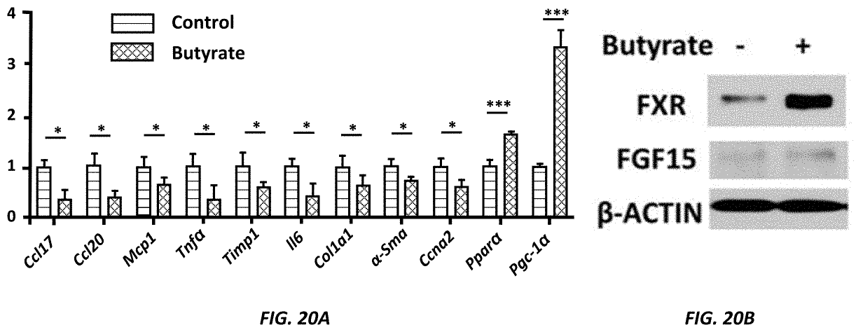

To avoid unwanted side effects of any drug, it is important to use chemicals normally present in the human body to treat disease. Our exciting data showed that food-derived product or bacteria-generated metabolites such as short chain fatty acid (SCFA) butyrate increased the mRNA and protein level of FGF21, FGFR1, and βKlotho in Huh7 cells (FIG. 19). Additionally, butyrate activated AMPK and ERK1/2 without changing the level of total AMPK and ERK1/2 as revealed by Western blot (FIG. 19). SCFAs are generated by microbial fermentation of indigestible dietary fiber. The most abundant SCFAs in the gut are acetate, propionate, and butyrate, which constitute 95% of the SCFAs (62). Of these 3 SCFAs, acetate causes hyperphagia and obesity, whereas butyrate protects against diet-induced obesity (92, 93). Butyrate is also present in dairy products such as cheese. Our recent publication revealed that butyrate supplementation is effective in treating NASH. In contrast, fecal transplantation of butyrate-deficient feces facilitates the development of NASH (39). Moreover, we showed that supplementation of butyrate in chow diet-fed healthy mice reduced the expression of many hepatic inflammation genes, fibrotic genes (Collagen 1a1 and α-smooth muscle actin), and proliferation gene (cyclin A2), and increased the expression of metabolism genes (Pparα and Pgc-1α) in mouse livers (FIG. 20A). Excitingly, butyrate supplementation also increased hepatic FXR as well as ileum FGF15 suggesting FXR activation (FIG. 20B). Moreover, in WD-fed obese mice, consistent with the published finding, butyrate improved insulin sensitivity (63).

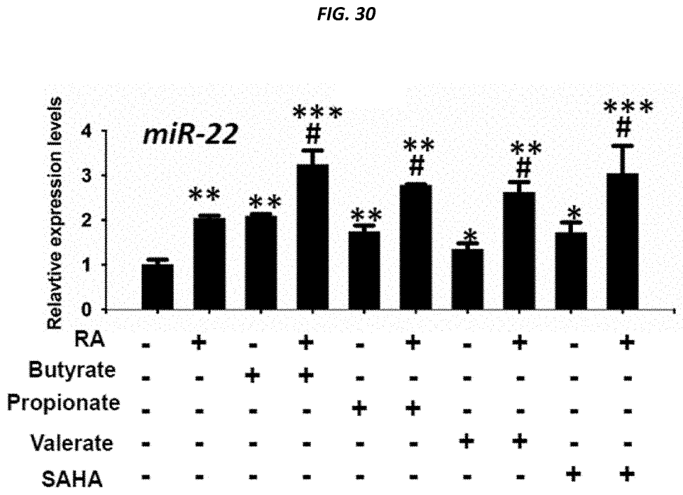

SAHA (suberanilohydroxamic acid), also known as Vorinostat, is a FDA-approved HDAC inhibitor used for cancer treatment. SCFAs including butyrate, propionate, and valerate are natural compounds that have HDAC inhibitory properties (64). Surprisingly, SAHA as well as all three SCFAs that have HDAC inhibitory effect were able to induce miR-22 in HCT116 cells, consistent with the finding obtained using Huh7 cells (FIGS. 19, 21, and 30). Those concentrations used for SCFAs were within physiological ranges. In contrast, SCFAs such as formate and acetate, which do not have HDAC inhibitory property, were not able to induce miR-22. These data revealed epigenetic effect in regulating the expression of miR-22, which will be studied in Aim 2.

### Overall Strategy

Based on the above presented data, we propose to study the beneficial metabolic effects of miR-22 inhibitors alone or in combination with FGF21, INT-747, and butyrate in lowering blood glucose level, improving insulin sensitivity as well as steatohepatitis treatment. Diet-induced obesity models will be used in both male and female mice. It is important to note that the WD used to generate preliminary data as well as in proposed experiments has a moderate amount of fat (21%) and high sucrose (34% of table sugar) resembling the diet commonly consumed by obese people. Our data showed that this WD reduced hepatic FGF21 as well as SIRT1 and generated fatty livers (FIG. 15A) (38, 39, 48). Our finding is consistent with published data that show simple sugar (sucrose) and saturated fat (palmitate) in a high-carbohydrate/moderate-fat diet induces lipogenesis and liver injury (65). The high fat diet frequently used to induce fatty liver usually has a larger amount of fat content (34%), but not elevated sucrose content compared with standard rodent chow diet. The ketogenic diet used for human weight loss reduces hepatic fat in human and increases hepatic FGF21 gene expression in rodents (66). Such ketogenic diet usually has 67% fat, but no sugar. Long-term consumption of all the above mentioned diets can induce fatty livers in rodents but may have different effects in regulating FGF21 signaling and glucose homeostasis. Thus, the proposed experiments will continue using the WD that has moderate amount of fat and high sucrose, which is most relevant to the WD that causes obesity in humans. To study the effect of miR-22, both mimics and inhibitors will be tested to ensure they have opposite metabolic outcomes in regulating diet-induced metabolic problems.

### Methodology and Analyses

### Animal Experiments

Male and female C57BL/6 wild type mice will be fed with differential control and Western diet (CD and WD, Harlan Teklad) after weaning (3-weeks old) (38,39,48). Based on our published data, when mice were 5-months old, WD-fed mice have elevated hepatic triglyceride and cholesterol with steatosis. In addition, WD-fed mice have increased fasting blood glucose and demonstrate glucose intolerance (48). Moreover, there is a gender difference in insulin sensitivity. CD-fed females are insulin sensitive compared with male counterparts. Because of this baseline difference, female mice were susceptible to the development of insulin resistance when they were on WD (48). When mice are 5-months old, they will be divided into subgroups listed in Table 3.

TABLE 3
Intervention subgroups.

| Male and Female | Intervention | miR-22 |
|---|---|---|
| CD and WD-fed | Control | control, miR-22 mimics, miR-22 |
| mice | | inhibitors |
| CD and WD-fed | FGF21 | control, miR-22 mimics, miR-22 |
| mice | | inhibitors |
| CD and WD-fed | INT-747 | control, miR-22 mimics, miR-22 |
| mice | | inhibitors |
| CD and WD-fed | Tributyrin | control, miR-22 mimics, miR-22 |
| mice | | inhibitors |

Together, there will be at least 48 groups and each group will have at least 12 mice. Mice will be treated by adenoviral-delivered FGF21, INT-747 (Abcam), and tributyrin (Sigma) or control in the presence or absence of adenoviral-delivered controls, miR-22 mimics, or miR-22 inhibitors.

The intervention methods are described below:

(1) Adenoviral-delivered FGF21 will be used alone or in combination with miR-22 inhibitors or mimics to treat mice on different diets. We use adenoviral-delivered FGF21 based on our promising data shown in FIG. 14. Recombinant FGF21 protein will not be used because miR-22 inhibitors work at post-transcriptional level to reduce FGF21. Inhibitors and mimics will be delivered when mice start receiving adenoviral-FGF21. We propose to use 2×10⁸ pfu/200 μl, once a week for 4 weeks. Such dose can effectively infect approximately 40% of cells in normal livers and fulminant hepatitis livers without affecting liver injury (67). This finding suggests that gene therapy with adenoviruses may be used efficiently and safely, even with severe liver disease. We will measure blood glucose level and insulin sensitivity to determine the effectiveness of treatments and adjust the dose and/or frequency of treatment as needed.
(2) INT-747 doses ranging from 5-30 mg/kg have been used for various disease models and are effective in improving insulin sensitivity, steatosis, obesity, gut permeability, and reducing inflammation (68-70). At 30 mg/kg/day, gavage, every 2 days for 4 weeks, INT-747 reduces hepatic inflammation and fibrosis (71). We will use this regimen for an initial trial. Dose and duration of treatment will be adjusted if there is a need.
(3) For butyrate, we will use tributyrin, which is a stable and rapidly absorbed prodrug of butyric acid. Such prodrug can be delivered to the distal digestive tract efficiently (72). Tributyrin will be delivered orally (1 g/kg/day) for 4 weeks. All the interventions will be done while mice continue their respective diet.

### Assays

Mice will be euthanized after one-month of interventions with and without miR-22 inhibitor or mimic treatment and subjected for the following assays.

(1) Morphology and liver function: Record body weight, food intake, and liver and fat pad weight. The degree of hepatic steatosis and liver histology will be monitored by Oil Red 0 staining as well as hematoxylin and eosin (H&E) staining. Liver histology will be evaluated. Liver function will be analyzed by serum ALT, AST, and ALP levels.
(2) Metabolism: Glucose and insulin tolerance tests will be conducted before the interventions and biweekly during the interventions as well as before euthanization. Serum and hepatic cholesterol, triglyceride, and FGF21 levels will be quantified. Hepatic RNA and protein will be extracted to quantify the level of FGF21, FGFR1, and βKlotho as well as SIRT1 and their downstream pathways for mitochondrial biogenesis (PGC1α), fatty acid uptake and oxidation (CPT1α), and ketogenesis (HMGCS2). The activation of energy sensor AMPK as well as ERK1/2 activation will be studied by Western blot. Moreover, the expression of genes regulating gluconeogenesis, i.e., Pepck, G6pase, and Fbp1 as well as fatty acid synthesis, uptake, and oxidation such as Fans, Screbp1, and Cyp4a11 in the liver will be studied by real-time PCR. Further, GLP1 secretion and serum PYY concentration will be quantified based on published methods to understand the mechanism for improved insulin sensitivity (61). Moreover, hepatic miR-22 level will be quantified by real-time PCR.
(3) Inflammatory and fibrotic signaling: Obesity is a chronic inflammatory status that impairs insulin sensitivity (73,74). The expression of genes regulating inflammatory signaling (IL-6, IL-10, IL-1β, IL-17, TNFα, Infγ, RORγ) as well as fibrosis (TGFβ, Col 1a1, Acta1) in the liver will be studied. Gut-derived signaling has an impact on hepatic inflammation (38). Thus, we will quantify serum lipopolysaccharide levels. We will also study epithelial integrity in the colon by immunostaining of Occludin as a measure of tight junction integrity. Moreover, we will also study the expression of tight junction protein ZO-1 and JAM-A, which affect gut permeability (75).
Statistical and Power analysis:

Hepatic fat content is the primary study end-point for the proposed animal study. Other outcomes are exploratory. A sample size of 12 mice per group will have a 95% power to detect at least 50% difference with standard deviation of 33% from the mean value, like the histological fat content data in our pilot study presented in FIG. 17. The primary analysis, separate for dietary effect, will be analysis of variance (ANOVA) for the 2×4×3 factorial design. The F test will use α=0.05 for overall Type I error. If the overall F test is significant, orthogonal contrasts will compare with and without interventions to validate the impact, and then test for miR-22 effects. Other outcome measures will use this same approach, with data transformed (e.g., logarithms) if necessary to satisfy assumptions.

### Expected Results and Alternative Approaches

We anticipate that miR-22 inhibitors will enhance the beneficial effects of FGF21, INT-747, and tributyrin in reducing blood glucose level, improving insulin sensitivity, as well as reducing hepatic fat and inflammation; whereas miR-22 mimics will dampen the intervention outcomes. miR-22 inhibitors are expected to increase exogenous FGF21 as well as endogenous FGF21, FGFR1, and SIRT1 thereby having added benefits. For INT-747, clinical trials that assess its usage in patients with NASH and T2DM have revealed promising effects. The results showed that 57% of INT-747-treated patients reached the primary endpoint of a ≥2-point improvement in NAFLD activity score without worsening fibrosis in comparison with 21% in the placebo group (p<0.01). Among patients with fibrosis, 41% of INT-747-treated patients had ≥1 stage of fibrosis improvement compared to 19% in the placebo group (p<0.05). Moreover, INT-747 improves insulin sensitivity, and reduced markers of liver inflammation and fibrosis (76). Our novel data showed that INT-747 increases hepatic FGF21, FGFR1, and βKlotho simultaneously indicating the significance of this pathway in contributing to the beneficial effects of INT-747. Reducing hepatic miR-22 should enhance the efficacy of INT-747.

It is fascinating that like INT-747, butyrate also increased hepatic FGF21, FGFR1, and βKlotho simultaneously. Moreover, butyrate increases hepatic FXR and ileal FGF15 indicating hepatic FXR activation. Whether butyrate can modify the acetylation of the FXR gene or protein will be studied in Aim 2. Because butyrate also induces hepatic miR-22, we are confident that miR-22 inhibitors can improve the effects of tributyrin as well. Although it is unlikely, if the data obtained are not as we expected, we will monitor the doses and the treatment frequency. Alternatively, based on the regulatory mechanisms of FGF21, FGFR1, and SIRT1 uncovered under Aim 2, we can design other novel approaches to enhance FGF21 and SIRT1 signaling in metabolic promised conditions.

The model we used, i.e., WD consumption, is relevant to humans, but is not expected to generate severe hepatic inflammation that has massive lymphocyte infiltration or advanced fibrosis. However, this model allows us to study the effect of those interventions in regulating inflammatory and fibrosis genes. Additionally, our data already showed that miR-22 mimic-treated mice had hepatic lymphocyte infiltration in addition to increased hepatic fat content (FIG. 17A). Thus, miR-22 mimics plus WD should produce advanced liver pathology, which can be a novel NASH model. Such new models allow us to study the undesirable metabolic effect of miR-22, which is found in human NASH (FIG. 15C). Increased lymphocyte infiltration found in miR-22-overexpressed livers may due to increased metabolic stress.

Alternatively, we will consider using other methods to generate advanced pathology to study the combined beneficial effects of miR-22 inhibitors plus INT-747 and other interventions. Those options include extending the WD feeding period (38,48), providing fructose to generate fibrosis (77), using stable isogenic cross breed of B6 and S129 in combination with diet (78), or using streptozotocin-nicotinamide to induce T2DM (79). Although methionine choline-deficient diet can produce advanced liver pathology, the clinical significance of this diet is questionable and will not be considered (80). Together, the model we proposed is able to give us an opportunity to access the detrimental metabolic effects of miR-22 as well as beneficial metabolic effect of miR-22 inhibitors. This large animal experiment can be labor intensive and time consuming. However, we are experienced in handling large animal trials as well as managing large datasets (38,39,48). We are confident that the generate data will lead to clinical trials because the studied compounds are either in clinical use, clinical trials, or natural food supplements.

Aim 2: Study the Mechanism by which miR-22 is Regulated that in Turn Affects Metabolism

### Rationale, Preliminary Data, and Overall Strategy

miR-22 is a metabolic inhibitor that reduces AMPK and deactivates ERK1/2. Thus, it is not surprising that miR-22 is also a tumor suppressor (43). Metabolic homeostasis can be maintained by coordinated induction of FGF21 and SIRT1 signaling as well as their negative regulator miR-22. This is important because for example, persistent SIRT1 activation can be tumorigenic. Most importantly, carcinogenic SIRT1 overexpression occurs at a post-transcriptional level since the mRNA level of SIRT1 is similar between normal liver and liver cancer tissue (81). Therefore, the expression of miR-22 needs to be tightly controlled to maintain homeostasis, and it is important to understand its underlying regulatory mechanism.

miR-22 is a Deacetylase Inhibitor

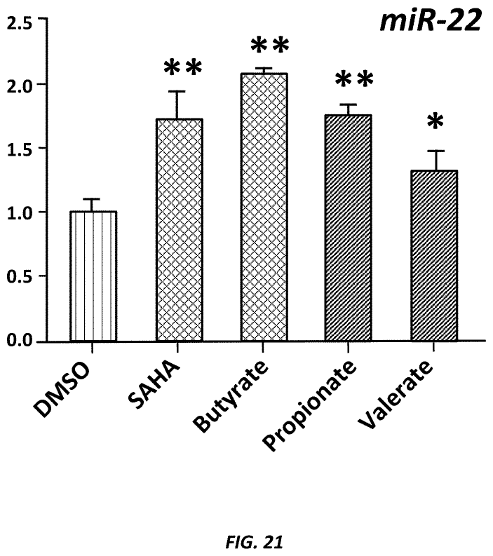

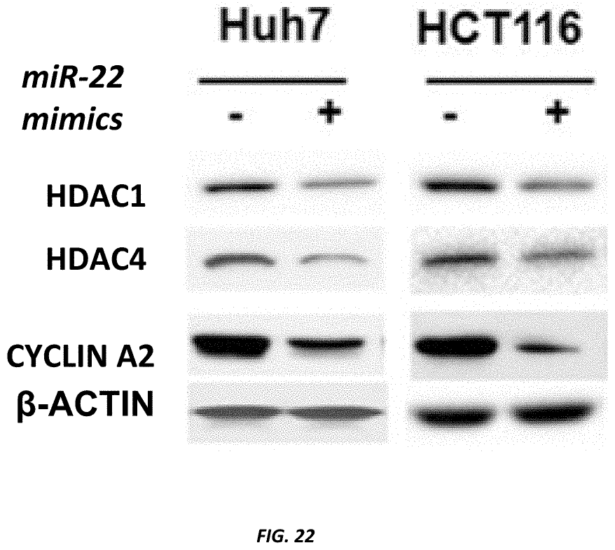

Our published data showed that miR-22 is transcriptionally regulated by FXR through direct binding to an invert repeat 1 (IR1) motif located at −1012 to −1025 base pair upstream from miR-22 (43). In contrast to FXR agonists, agonists for other nuclear receptors inducing PPARα, PXR, RARβ, and CAR were not able to induce miR-22 with the exception of vitamin D3. In addition to transcriptional regulation controlled by FXR, we propose to study epigenetic regulation of miR-22 because our novel data showed that miR-22 level was inducible by HDAC inhibitors including SAHA (FIG. 21). It is fascinating to uncover that miR-22 itself is a HDAC inhibitor as miR-22 reduces deacetylases such as HDAC4 and SIRT1 in different cell types (42,82,83). Additionally, our data showed for the first time that miR-22 also reduced HDAC1. In human Huh7 and human colon HCT116 cells, miR-22 reduced the protein level of HDAC1 and HDAC4 as well as CYCLIN A2. CYCLIN A2 is a known miR-22 target, which was included as a positive control (FIG. 22). Furthermore, by sequence alignment, we have found that miR-22 also pairs with the 3′UTR of HDAC6, HDAC8, and HDAC11. There are four classes of HDACs in mammalian cells. SAHA and butyrate commonly inhibit Class I (HDAC1, 2, 3, and 8) and Class IIa (HDAC4, 5, 7, and 9). Class III includes SIRT1-7. Our experimental data showed that miR-22 reduces HDAC1, HDAC4, and SIRT1 (FIGS. 16 and 22). Together, miR-22 likely inhibits all Classes of HDACs indicating its substantial role in reducing deacetylases.

### Possible Consequences of Deacetylase Reduction

Increased histone acetylation plays a pivotal role in transcriptional activation. Thus, by silencing deacetylases, miR-22 induction may lead to increased transcriptional machinery of miR-22 itself. This is supported by the finding that SAHA and butyrate were able to induce miR-22. Additionally, miR-22-silenced HDACs may also contribute to the increased FXR expression evidenced by the effect of butyrate in inducing FXR (FIG. 20B). Moreover, miR-22-silenced HDACs may have a role in transcriptional activation of FXR leading to increased expression of FXR-target genes like Fgf21, Fgfr1, and βKlotho (FIGS. 18 and 19). These expectations are also supported by the effect of FXR agonist and butyrate concomitantly increased the mRNA and protein level of all these genes.

In addition to histone modification, miR-22-reduced deacetylases may induce protein acetylation. It has been shown that FXR is a target of SIRT1. Lysine 217 of FXR is the major acetylation site targeted by SIRT1. Acetylation of FXR increases its stability but inhibits heterodimerization with RXRα and DNA binding thereby leading to reduced transactivation activity (84). We hypothesize that in a metabolically compromised condition, increased miR-22 via protein acetylation may inactivate FXR transcriptional activity leading to steatosis. Thus, acetylation is both an upstream regulatory mechanism for miR-22 expression as well as a downstream effect of miR-22 forming a self-regulatory loop. These hypotheses will be studied in cell culture models as well as mice used in Aim 1.

Other Downstream Effect of miR-22

Another goal of this Aim is to study the underlying mechanism by which miR-22 reduces FGF21 and FGFR1, which has never been documented before. Because SIRT1 is a known miR-22 direct target (85-87), this experiment does not need to be repeated. By sequence alignment, our initial observation revealed that miR-22 pairs with the 3′UTR of the FGF21, but not the FGFR1. Therefore, it is possible that miR-22 directly targets FGF21. Alternatively, miR-22-silenced HDACs or other targets may indirectly have an impact on their expression by regulating the transcriptional activity of FXR. It is critically important to gain more understanding of how FGF21, FGFR1, and βKlotho are coordinately regulated in a metabolically active state driven by FXR activation and butyrate treatment. Such information will lead to novel strategies to activate AMPK. Thus, we will study the expression and regulation of miR-22 and FGF21 signaling in cells and mice.

### Overall Strategy:

To have a global understanding of the effect of miR-22 on HDAC activity, we will knockdown as well as overexpress miR-22 in human liver Huh7 and Hep3B cell lines followed by HDAC activity assays using ELISA. Using two lines will generate reproducible results. The HDACs that have altered activity due to differential miR-22 expression will be further validated by Western blot. Moreover, we will study whether those HDACs are miR-22 direct targets. To determine whether miR-22-silenced HDACs are also involved in regulating miR-22, the occupancy of those HDACs in the regulatory region of the miR-22 will be studied using ChIP-qPCR in cells treated with and without miR-22 inducers or with and without miR-22 overexpression. Additionally, we will study the occupancy of those HDACs in the miR-22 regulatory region in metabolically active livers as well as fatty livers of mice used in Aim 1. Such approaches will help us understand the mechanism by which miR-22 is regulated in a normal state, in response to metabolic stimulators, as well as in metabolic compromised conditions.

To understand the impact of miR-22-reduced deacetylases in FXR gene expression and transcriptional activation, we propose to study histone modification of the FXR gene in butyrate and SAHA-treated Huh7 and Hep3B cells as well as miR-22 overexpressed or knockdown cells. The occupancy of the HDACs targeted by miR-22 will be studied in the regulatory region of the FXR genes by ChIP-qPCR. Moreover, we will study whether the effect of butyrate in inducing FGF21, FGFR1, and βKlotho is FXR-dependent by knockdown and overexpressing FXR in butyrate-treated cells. Furthermore, FXR protein acetylation will be analyzed in cell and animal models. Such approaches determine if miR-22 activation is involved in both transcriptional activation of FXR to increase AMPK activity, and acetylation of FXR protein leading to FXR inactivation thereby lacking FGF21, FGFR1, and βKlotho induction in metabolic active and compromised state, respectively.

To study other downstream effects of miR-22, we will determine whether miR-22 can directly target FGF21 and FGFR1. Together, the proposed comprehensive approaches will allow us to uncover novel mechanism by which those master metabolic regulators are expressed in metabolic active and compromised states.

### Methodology and Analyses

Uncover miR-22-Silenced Deacetylases

All the proposed cell experiments will be done using at least two cell lines derived from human livers such as Huh7 and Hep3B. We will uncover HDACs that can be reduced by miR-22. Cells will be infected by adenoviral-scramble miRNA-GFP (green fluorescent protein), which will serve as the control, or adenoviral-miR-22-3P-GFP mimics and inhibitors. Thus, the delivery and expression can be monitored by the detection of green fluorescence. The HDAC activity in the cells that have differential miR-22 levels will be analyzed by HDAC ELISA assays (Reaction Biology Co.) and confirmed by Western blot. Additionally, we will treat the cells with INT-747, butyrate, and SAHA to determine whether the level of specific HDACs is reduced based on miR-22 induction. SAHA will be included in most cell culture experiments to compare with the HDAC inhibitory property of butyrate. Prodrug tributyrin will only be used in mice to effectively deliver butyrate to the distal digestive tract and liver. For the cell culture experiments, there is no need to use tributyrin. Furthermore, miR-22 inhibitors will be used to block the effect of INT-747 and butyrate in reducing HDACs thereby demonstrating miR-22 dependency. These approaches will establish the consequential relationship of metabolism activation induced by INT-747 and butyrate, miR-22 induction, and specific HDAC silencing.

Validation of miR-22-Targeted HDACs

We will clone 3′UTR of those HDACs that can be silenced by miR-22 into the psiCHECK2 plasmid for transfection with either miR-22 mimics or scramble controls followed by treatment with or without INT-747, butyrate, or SAHA in Huh7 and Hep3B cells. Then, luciferase assay will be performed. The reduced luciferase activity due to the presence of miR-22 mimics or its inducers suggests binding. Additionally, single nucleotide site-directed mutagenesis study will be performed to confirm binding specificity. The potential candidates that will be studied are HDAC1, HDAC6, HDAC8, and HDAC11 as justified above. Moreover, the expression level of miR-22 and its targeted HDACs will be studied in the mouse livers used in Aim 1 by real-time PCR and Western blot.

Epigenetic Regulation of miR-22

To study if miR-22-silenced HDACs regulate miR-22 expression, cells will be treated with and without miR-22 inducers or infected with and without adenoviral-miR-22 or miR-22 inhibitors. Chromatin prepared from those cells will be used to study the occupancy of HDACs in the regulatory region of miR-22 by ChIP-qPCR using antibodies specific for miR-22-targeted HDACs. Moreover, anti-histone H3 and H4 antibodies will be used for ChIP-qPCR to access how acetylation pattern of miR-22 changes in response to miR-22 inducer treatment or miR-22 over and under-expression. Furthermore, the same approaches will be used in mouse livers studied under Aim 1. Since miR-22 is induced by both metabolic stimulators and steatosis, it is likely that different mechanisms are involved for its induction.

### Epigenetic Impact on FXR Expression and Transcriptional Activation

Both FXR agonist and butyrate increased FGF21, FGFR1, and βKlotho at mRNA and protein level. By knockout and overexpression of FXR in Huh7 and Hep3B cells, we will analyze if the effect of butyrate in inducing FGF21, FGFR1, and βKlotho is in part FXR-dependent. The acetylation pattern of the FXR gene will be studied in cells treated with and without butyrate and SAHA or with miR-22 over and under expression. Anti-histone H3 and H4 antibodies will be used for ChIP-qPCR to access how acetylation pattern of the FXR gene changes. The potential recruitment of miR-22-silenced HDACs in the regulatory region of the FXR gene will be examined by ChIP-qPCR using anti-HDAC antibodies. Moreover, the potential interaction of those HDACs with FXR protein will be studied by immunoprecipitation (IP) using specific anti-HDAC antibodies followed by Western blot using anti-FXR antibody. If FXR and deacetylase interact, we will study the acetylation pattern using anti-acetyl antibody for IP followed by performing Western blot using anti-FXR antibody. Moreover, FXR transcriptional activity can be monitored by studying the expression level of its regulated genes as well as the recruitment of FXR to the regulatory region of its target genes such as FGF21, FGFR1, and βKlotho. The same experiments will be performed in mouse livers used in Aim 1.

Study the Mechanisms by which miR-22 Silences FGF21 and FGFR1

In vitro functional assays will be conducted essentially using the same methods described to study miR-22 silencing of HDACs. We will clone the 3′UTR of the FGF21 and FGFR1 into the psiCHECK2 plasmid for co-transfection with either miR-22 mimics or scramble controls in Hep3B and Huh7 cells followed by luciferase assays. The transfected cells will be treated with and without miR-22 inducers such as INT-747, butyrate, and SAHA as well followed by luciferase assays. miR-22 and miR-22 inducers should reduce luciferase reporter activity if miR-22 binds the 3′UTR of a given gene.

### Expected Results and Alternative Approaches

Our published data show that the level of miR-22 is reduced in FXR KO mice and induced by FXR agonists through a FXR-dependent manner (43). In addition, our preliminary data showed the ability of HDAC inhibitors in inducing miR-22. Thus, we anticipate that the expression of miR-22 is regulated transcriptionally by FXR and epigenetically through acetylation. Additionally, since butyrate can concomitantly increase FXR as well as miR-22, it is very likely that butyrate-induced miR-22 is in part FXR-dependent. We also anticipate that butyrate or miR-22-silenced HDACs are involved in transcriptional regulation the FXR gene as well as miR-22 itself. Thus, in response to INT-747 and butyrate treatment, the induced miR-22 may reduce the recruitment of HDACs in the regulatory region the FXR gene and miR-22 leading to increased transcription and expression. However, in a metabolically compromised condition, overexpressed miR-22 may silence specific protein deacetylase such as SIRT1 that potentially can increase the acetylation of FXR protein lead to FXR deactivation. This scenario is supported by the finding that resveratrol-activated SIRT1 reduces acetylated-FXR and lead to FXR transcriptional activation (84). The specific HDACs recruited to interact with the FXR gene and protein will be uncovered in cells and validated in mouse livers. If the effect of butyrate in inducing FGF21, FGFR1, and βKlotho is FXR independent, alternative approaches are to study acetylation pattern of the FGF21, FGFR1, and βKlotho genes. The effect of PPARα will be considered since PPARα activation induces FGF21. It is not clear whether PPARα agonist is able to induce the expression of all three genes. Moreover, we will consider studying the combination effect of INT-747 and butyrate or the combination effect of INT-747 plus SAHA. If combination treatment does not further increase gene expression in comparison with single treatment, it suggests these two chemicals regulate overlapping pathways. It is important to note that in addition to HDAC inhibition property, butyrate has many other biological effects. Those effects can be mediated through butyrate receptor GPR109, which is a membrane receptor. Since the focus of Aim 2 is to understand mechanism for gene and protein expression, we only discuss the HDAC inhibitory effect of butyrate.

miR-22 is also induced in the fatty livers, where FGF21 and SIRT1 signaling is reduced. Overexpressed miR-22 contributes to the development of steatosis as evidenced by the data presented in FIGS. 15 and 17. We anticipate that distinct mechanisms are involved in miR-22 induction in response to metabolic activation or inactivation because FXR is unlikely to be activated in the fatty liver. We anticipate that epigenetic regulation may play a key role for miR-22 induction in metabolic compromised conditions. The proposed experiments will delineate whether the same HDACs are involved in inducing miR-22 in metabolic active and compromised conditions in mice.

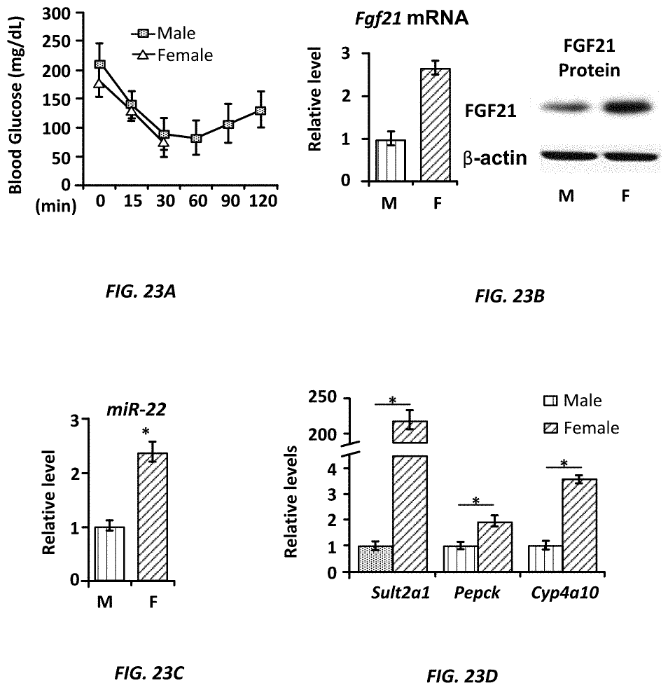

Gender difference in metabolism is well-known (88-90). Our data showed that age and diet-matched female mice were highly sensitive to insulin compared with their male counterparts (FIG. 23). Female mice became hypoglycemic 60 minutes after insulin injection and required glucose injection to rescue them (FIG. 23A), whereas age-matched male mice did not have this issue. Surprisingly, hepatic FGF21 mRNA and protein as well as hepatic miR-22 were also higher in female than male mice (FIG. 23B). Thus, it seems that elevated FGF21 found in female mouse livers requires more miR-22 to reduce it thereby metabolic homeostasis can be maintained in a gender-specific way. Together, coordinated elevation of hepatic FGF21 and miR-22 is associated with more optimal metabolic homeostasis in healthy condition. Furthermore, our published data showed that sex difference in steatosis is FXR-dependent (48). Moreover, bile acid synthesis and homeostasis are also sex different (48,91). Because FXR is a key FGF21 regulator and gender dimorphism in FGF21 homeostasis has not been thoroughly examined, the proposed approaches provide a rare opportunity to study whether sex difference in miR-22-regulated signaling can account for sex difference in metabolism or susceptibility in metabolic disease development. Based on the phenotype of mice, gene expression profiles, and perhaps differential recruitment levels of HDACs to the regulatory regions of the studied genes, we may elucidate whether the differential expression of miR-22 found in two sexes accounts for the sex difference in metabolism. If this is the case, we will consider performing castration and ovariectomy to determine whether such differences can be abolished.

The major challenge of Aim 2 is the large sample size with many experimental groups that will be subjected for various molecular biology analyses. We will focus on those mice that have extreme phenotypes based on the severity of steatosis, glucose level, and insulin sensitivity to understand the differential regulatory mechanisms.

### REFERENCES

1. Hinds, T. D., Jr., Sodhi, K., Meadows, C., Fedorova, L., Puri, N., Kim, D. H., Peterson, S. J., Shapiro, J., Abraham, N. G. & Kappas, A. (2014) Increased HO-1 levels ameliorate fatty liver development through a reduction of heme and recruitment of FGF21, Obesity (Silver Spring, Md.). 22, 705-12.
2. So, W. Y. & Leung, P. S. (2016) Fibroblast Growth Factor 21 As an Emerging Therapeutic Target for Type 2 Diabetes Mellitus, Medicinal research reviews. 36, 672-704.
3. Staiger, H., Keuper, M., Berti, L., Hrabe de Angelis, M. & Haring, H. U. (2017) Fibroblast Growth Factor 21-Metabolic Role in Mice and Men, Endocrine reviews. 38, 468-488.
4. Woo, Y. C., Xu, A., Wang, Y. & Lam, K. S. (2013) Fibroblast growth factor 21 as an emerging metabolic regulator: clinical perspectives, Clinical endocrinology. 78, 489-96.
5. Cuevas-Ramos, D., Almeda-Valdes, P., Aguilar-Salinas, C. A., Cuevas-Ramos, G., Cuevas-Sosa, A. A. & Gomez-Perez, F. J. (2009) The role of fibroblast growth factor 21 (FGF21) on energy balance, glucose and lipid metabolism, Current diabetes reviews. 5, 216-20.
6. Potthoff, M. J., Inagaki, T., Satapati, S., Ding, X., He, T., Goetz, R., Mohammadi, M., Finck, B. N., Mangelsdorf, D. J., Kliewer, S. A. & Burgess, S. C. (2009) FGF21 induces PGC-1alpha and regulates carbohydrate and fatty acid metabolism during the adaptive starvation response, Proc Natl Acad Sci USA. 106, 10853-8.
7. Zhou, G., Myers, R., Li, Y., Chen, Y., Shen, X., Fenyk-Melody, J., Wu, M., Ventre, J., Doebber, T., Fujii, N., Musi, N., Hirshman, M. F., Goodyear, L. J. & Moller, D. E. (2001) Role of AMP-activated protein kinase in mechanism of metformin action, J Clin Invest. 108, 1167-74.
8. Saha, A. K., Avilucea, P. R., Ye, J. M., Assifi, M. M., Kraegen, E. W. & Ruderman, N. B. (2004) Pioglitazone treatment activates AMP-activated protein kinase in rat liver and adipose tissue in vivo, Biochem Biophys Res Commun. 314, 580-5.
9. Ruderman, N. B., Carling, D., Prentki, M. & Cacicedo, J. M. (2013) AMPK, insulin resistance, and the metabolic syndrome, J Clin Invest. 123, 2764-72.
10. Li, Y., Wong, K., Giles, A., Jiang, J., Lee, J. W., Adams, A. C., Kharitonenkov, A., Yang, Q., Gao, B., Guarente, L. & Zang, M. (2014) Hepatic SIRT1 attenuates hepatic steatosis and controls energy balance in mice by inducing fibroblast growth factor 21, Gastroenterology. 146, 539-49.e7.
11. Ge, X., Chen, C., Hui, X., Wang, Y., Lam, K. S. & Xu, A. (2011) Fibroblast growth factor 21 induces glucose transporter-1 expression through activation of the serum response factor/Ets-like protein-1 in adipocytes, The Journal of biological chemistry. 286, 34533-41.
12. Domouzoglou, E. M., Fisher, F. M., Astapova, I., Fox, E. C., Kharitonenkov, A., Flier, J. S., Hollenberg, A. N. & Maratos-Flier, E. (2014) Fibroblast growth factor 21 and thyroid hormone show mutual regulatory dependency but have independent actions in vivo, Endocrinology. 155, 2031-40.
13. Liu, H. X., Hu, Y. & Wan, Y. J. (2016) Microbiota and bile acid profiles in retinoic acid-primed mice that exhibit accelerated liver regeneration, Oncotarget. 7, 1096-106.
14. Liu, H. X., Hu, Y., French, S. W., Gonzalez, F. J. & Wan, Y. J. (2015) Forced expression of fibroblast growth factor 21 reverses the sustained impairment of liver regeneration in hPPARalpha(PAC) mice due to dysregulated bile acid synthesis, Oncotarget. 6, 9686-700.
15. Zhang, Y., Xie, Y., Berglund, E. D., Coate, K. C., He, T. T., Katafuchi, T., Xiao, G., Potthoff, M. J., Wei, W., Wan, Y., Yu, R. T., Evans, R. M., Kliewer, S. A. & Mangelsdorf, D. J. (2012) The starvation hormone, fibroblast growth factor-21, extends lifespan in mice, eLife. 1, e00065.
16. Fontana, L., Zhao, E., Amir, M., Dong, H., Tanaka, K. & Czaja, M. J. (2013) Aging promotes the development of diet-induced murine steatohepatitis but not steatosis, Hepatology (Baltimore, Md.). 57, 995-1004.
17. Chau, M. D., Gao, J., Yang, Q., Wu, Z. & Gromada, J. (2010) Fibroblast growth factor 21 regulates energy metabolism by activating the AMPK-SIRT1-PGC-1alpha pathway, Proc Natl Acad Sci USA. 107, 12553-8.
18. Wente, W., Efanov, A. M., Brenner, M., Kharitonenkov, A., Koster, A., Sandusky, G. E., Sewing, S., Treinies, I., Zitzer, H. & Gromada, J. (2006) Fibroblast growth factor-21 improves pancreatic beta-cell function and survival by activation of extracellular signal-regulated kinase 1/2 and Akt signaling pathways, Diabetes. 55, 2470-8.
19. Escande, C., Chini, C. C., Nin, V., Dykhouse, K. M., Novak, C. M., Levine, J., van Deursen, J., Gores, G. J., Chen, J., Lou, Z. & Chini, E. N. (2010) Deleted in breast cancer-1 regulates SIRT1 activity and contributes to high-fat diet-induced liver steatosis in mice, J Clin Invest. 120, 545-58.
20. Kitada, M. & Koya, D. (2013) SIRT1 in Type 2 Diabetes: Mechanisms and Therapeutic Potential, Diabetes & metabolism journal. 37, 315-25.
21. Sooyeon, L., Go, K. L. & Kim, J. S. (2016) Deacetylation of mitofusin-2 by sirtuin-1: A critical event in cell survival after ischemia, Molecular & cellular oncology. 3, e1087452.
22. Yin, H., Hu, M., Liang, X., Ajmo, J. M., Li, X., Bataller, R., Odena, G., Stevens, S. M., Jr. & You, M. (2014) Deletion of SIRT1 from hepatocytes in mice disrupts lipin-1 signaling and aggravates alcoholic fatty liver, Gastroenterology. 146, 801-11.
23. Salminen, A. & Kaarniranta, K. (2009) SIRT1: regulation of longevity via autophagy, Cellular signalling. 21, 1356-60.
24. Boutant, M. & Canto, C. (2014) SIRT1 metabolic actions: Integrating recent advances from mouse models, Molecular metabolism. 3, 5-18.
25. Canto, C. & Auwerx, J. (2009) PGC-1alpha, SIRT1 and AMPK, an energy sensing network that controls energy expenditure, Current opinion in lipidology. 20, 98-105.
26. Cyphert, H. A., Ge, X., Kohan, A. B., Salati, L. M., Zhang, Y. & Hillgartner, F. B. (2012) Activation of the farnesoid X receptor induces hepatic expression and secretion of fibroblast growth factor 21, J Biol Chem. 287, 25123-38.
27. Galman, C., Lundasen, T., Kharitonenkov, A., Bina, H. A., Eriksson, M., Hafstrom, I., Dahlin, M., Amark, P., Angelin, B. & Rudling, M. (2008) The circulating metabolic regulator FGF21 is induced by prolonged fasting and PPARalpha activation in man, Cell Metab. 8, 169-74.
28. Revollo, J. R. & Li, X. (2013) The ways and means that fine tune Sirtl activity, Trends in biochemical sciences. 38, 160-7.
29. Sa-Nguanmoo, P., Chattipakorn, N. & Chattipakorn, S. C. (2016) Potential roles of fibroblast growth factor 21 in the brain, Metabolic brain disease. 31, 239-48.
30. Nishimura, T., Nakatake, Y., Konishi, M. & Itoh, N. (2000) Identification of a novel FGF, FGF-21, preferentially expressed in the liver, Biochim Biophys Acta. 1492, 203-6.
31. Kharitonenkov, A. & Shanafelt, A. B. (2009) FGF21: a novel prospect for the treatment of metabolic diseases, Current opinion in investigational drugs. 10, 359-64.
32. Fon Tacer, K., Bookout, A. L., Ding, X., Kurosu, H., John, G. B., Wang, L., Goetz, R., Mohammadi, M., Kuro-o, M., Mangelsdorf, D. J. & Kliewer, S. A. (2010) Research resource: Comprehensive expression atlas of the fibroblast growth factor system in adult mouse, Molecular endocrinology (Baltimore, Md.). 24, 2050-64.
33. Liu, S. Q., Roberts, D., Kharitonenkov, A., Zhang, B., Hanson, S. M., Li, Y. C., Zhang, L. Q. & Wu, Y. H. (2013) Endocrine protection of ischemic myocardium by FGF21 from the liver and adipose tissue, Scientific reports. 3, 2767.
34. Yang, F., Huang, X., Yi, T., Yen, Y., Moore, D. D. & Huang, W. (2007) Spontaneous development of liver tumors in the absence of the bile acid receptor farnesoid X receptor, Cancer Res. 67, 863-7.
35. Kim, I., Morimura, K., Shah, Y., Yang, Q., Ward, J. M. & Gonzalez, F. J. (2007) Spontaneous hepatocarcinogenesis in farnesoid X receptor-null mice, Carcinogenesis. 28, 940-6.
36. Wolfe, A., Thomas, A., Edwards, G., Jaseja, R., Guo, G. L. & Apte, U. (2011) Increased activation of the Wnt/beta-catenin pathway in spontaneous hepatocellular carcinoma observed in farnesoid X receptor knockout mice, The Journal of pharmacology and experimental therapeutics. 338, 12-21.
37. Li, G., Kong, B., Zhu, Y., Zhan, L., Williams, J. A., Tawfik, O., Kassel, K. M., Luyendyk, J. P., Wang, L. & Guo, G. L. (2013) Small heterodimer partner overexpression partially protects against liver tumor development in farnesoid X receptor knockout mice, Toxicology and applied pharmacology. 272, 299-305.
38. Jena, P. K., Sheng, L., Liu, H. X., Kalanetra, K. M., Mirsoian, A., Murphy, W. J., French, S. W., Krishnan, V. V., Mills, D. A. & Wan, Y. Y. (2017) Western Diet-Induced Dysbiosis in Farnesoid X Receptor Knockout Mice Causes Persistent Hepatic Inflammation after Antibiotic Treatment, The American journal of pathology. 187, 1800-1813.
39. Sheng, L., Jena, P. K., Hu, Y., Liu, H. X., Nagar, N., Kalanetra, K. M., French, S. W., French, S. W., Mills, D. A. & Wan, Y. Y. (2017) Hepatic inflammation caused by dysregulated bile acid synthesis is reversible by butyrate supplementation, J Pathol.
40. Jiang, C., Xie, C., Lv, Y., Li, J., Krausz, K. W., Shi, J., Brocker, C. N., Desai, D., Amin, S. G., Bisson, W. H., Liu, Y., Gavrilova, O., Patterson, A. D. & Gonzalez, F. J. (2015) Intestine-selective farnesoid X receptor inhibition improves obesity-related metabolic dysfunction, Nature communications. 6, 10166.
41. Jiang, C., Xie, C., Li, F., Zhang, L., Nichols, R. G., Krausz, K. W., Cai, J., Qi, Y., Fang, Z. Z., Takahashi, S., Tanaka, N., Desai, D., Amin, S. G., Albert, I., Patterson, A. D. & Gonzalez, F. J. (2015) Intestinal farnesoid X receptor signaling promotes nonalcoholic fatty liver disease, J Clin Invest. 125, 386-402.
42. Wang, J., Li, Y., Ding, M., Zhang, H., Xu, X. & Tang, J. (2017) Molecular mechanisms and clinical applications of miR-22 in regulating malignant progression in human cancer (Review), International journal of oncology. 50, 345-355.
43. Yang, F., Hu, Y., Liu, H. X. & Wan, Y. J. (2015) MiR-22-silenced cyclin A expression in colon and liver cancer cells is regulated by bile acid receptor, J Biol Chem. 290, 6507-15.
44. Chen, J., Zhang, B., Wong, N., Lo, A. W., To, K. F., Chan, A. W., Ng, M. H., Ho, C. Y., Cheng, S. H., Lai, P. B., Yu, J., Ng, H. K., Ling, M. T., Huang, A. L., Cai, X. F. & Ko, B. C. (2011) Sirtuin 1 is upregulated in a subset of hepatocellular carcinomas where it is essential for telomere maintenance and tumor cell growth, Cancer Res. 71, 4138-49.
45. Lin, Z. & Fang, D. (2013) The Roles of SIRT1 in Cancer, Genes & cancer. 4, 97-104.
46. Hao, C., Zhu, P. X., Yang, X., Han, Z. P., Jiang, J. H., Zong, C., Zhang, X. G., Liu, W. T., Zhao, Q. D., Fan, T. T., Zhang, L. & Wei, L. X. (2014) Overexpression of SIRT1 promotes metastasis through epithelial-mesenchymal transition in hepatocellular carcinoma, BMC cancer. 14, 978.
47. Jiang, H., Zhang, X., Tao, Y., Shan, L., Jiang, Q., Yu, Y., Cai, F. & Ma, L. (2017) Prognostic and clinicopathologic significance of SIRT1 expression in hepatocellular carcinoma, Oncotarget. 8, 52357-52365.
48. Sheng, L., Jena, P. K., Liu, H. X., Kalanetra, K. M., Gonzalez, F. J., French, S. W., Krishnan, V. V., Mills, D. A. & Wan, Y. Y. (2017) Gender Differences in Bile Acids and Microbiota in Relationship with Gender Dissimilarity in Steatosis Induced by Diet and FXR Inactivation, Scientific reports. 7, 1748.
49. Liu, X., Zhang, P., Martin, R. C., Cui, G., Wang, G., Tan, Y., Cai, L., Lv, G. & Li, Y. (2016) Lack of fibroblast growth factor 21 accelerates metabolic liver injury characterized by steatohepatities in mice, American journal of cancer research. 6, 1011-25.
50. Postic, C., Shiota, M., Niswender, K. D., Jetton, T. L., Chen, Y., Moates, J. M., Shelton, K. D., Lindner, J., Cherrington, A. D. & Magnuson, M. A. (1999) Dual roles for glucokinase in glucose homeostasis as determined by liver and pancreatic beta cell-specific gene knock-outs using Cre recombinase, J Biol Chem. 274, 305-15.
51. Kharitonenkov, A., Shiyanova, T. L., Koester, A., Ford, A. M., Micanovic, R., Galbreath, E. J., Sandusky, G. E., Hammond, L. J., Moyers, J. S., Owens, R. A., Gromada, J., Brozinick, J. T., Hawkins, E. D., Wroblewski, V. J., Li, D. S., Mehrbod, F., Jaskunas, S. R. & Shanafelt, A. B. (2005) FGF-21 as a novel metabolic regulator, J Clin Invest. 115, 1627-35.
52. Fisher, F. M., Chui, P. C., Antonellis, P. J., Bina, H. A., Kharitonenkov, A., Flier, J. S. & Maratos-Flier, E. (2010) Obesity is a fibroblast growth factor 21 (FGF21)-resistant state, Diabetes. 59, 2781-9.
53. Zhang, Y. (2010) Farnesoid X receptor-Acting through bile acids to treat metabolic disorders, Drugs of the future. 35, 635-642.
54. Li, T. & Chiang, J. Y. (2014) Bile acid signaling in metabolic disease and drug therapy, Pharmacological reviews. 66, 948-83.
55. Kim, D. H., Xiao, Z., Kwon, S., Sun, X., Ryerson, D., Tkac, D., Ma, P., Wu, S. Y., Chiang, C. M., Zhou, E., Xu, H. E., Palvimo, J. J., Chen, L. F., Kemper, B. & Kemper, J. K. (2015) A dysregulated acetyl/SUMO switch of FXR promotes hepatic inflammation in obesity, The EMBO journal. 34, 184-99.
56. Porez, G., Prawitt, J., Gross, B. & Staels, B. (2012) Bile acid receptors as targets for the treatment of dyslipidemia and cardiovascular disease, Journal of lipid research. 53, 1723-37.
57. Matsubara, T., Li, F. & Gonzalez, F. J. (2013) FXR signaling in the enterohepatic system, Molecular and cellular endocrinology. 368, 17-29.
58. Xu, Y., Li, F., Zalzala, M., Xu, J., Gonzalez, F. J., Adorini, L., Lee, Y. K., Yin, L. & Zhang, Y. (2016) Farnesoid X receptor activation increases reverse cholesterol transport by modulating bile acid composition and cholesterol absorption in mice, Hepatology (Baltimore, Md.). 64, 1072-85.
59. Bowlus, C. L. (2016) Obeticholic acid for the treatment of primary biliary cholangitis in adult patients: clinical utility and patient selection, Hepatic medicine: evidence and research. 8, 89-95.
60. Neuschwander-Tetri, B. A., Loomba, R., Sanyal, A. J., Lavine, J. E., Van Natta, M. L., Abdelmalek, M. F., Chalasani, N., Dasarathy, S., Diehl, A. M., Hameed, B., Kowdley, K. V., McCullough, A., Terrault, N., Clark, J. M., Tonascia, J., Brunt, E. M., Kleiner, D. E. & Doo, E. (2015) Farnesoid X nuclear receptor ligand obeticholic acid for non-cirrhotic, non-alcoholic steatohepatitis (FLINT): a multicentre, randomised, placebo-controlled trial, Lancet. 385, 956-65.
61. Pathak, P., Liu, H., Boehme, S., Xie, C., Krausz, K. W., Gonzalez, F. & Chiang, J. Y. L. (2017) Farnesoid X receptor induces Takeda G-protein receptor 5 cross-talk to regulate bile acid synthesis and hepatic metabolism, J Biol Chem. 292, 11055-11069.
62. Rios-Covian, D., Ruas-Madiedo, P., Margolles, A., Gueimonde, M., de Los Reyes-Gavilan, C. G. & Salazar, N. (2016) Intestinal Short Chain Fatty Acids and their Link with Diet and Human Health, Frontiers in microbiology. 7, 185.
63. Gao, Z., Yin, J., Zhang, J., Ward, R. E., Martin, R. J., Lefevre, M., Cefalu, W. T. & Ye, J. (2009) Butyrate improves insulin sensitivity and increases energy expenditure in mice, Diabetes. 58, 1509-17.
64. Vinolo, M. A., Rodrigues, H. G., Nachbar, R. T. & Curi, R. (2011) Regulation of inflammation by short chain fatty acids, Nutrients. 3, 858-76.
65. Pierce, A. A., Duwaerts, C. C., Soon, R. K., Siao, K., Grenert, J. P., Fitch, M., Hellerstein, M. K., Beysen, C., Turner, S. M. & Maher, J. J. (2016) Isocaloric manipulation of macronutrients within a high-carbohydrate/moderate-fat diet induces unique effects on hepatic lipogenesis, steatosis and liver injury, The Journal of nutritional biochemistry. 29, 12-20.
66. Douris, N., Desai, B. N., Fisher, F. M., Cisu, T., Fowler, A. J., Zarebidaki, E., Nguyen, N. L. T., Morgan, D. A., Bartness, T. J., Rahmouni, K., Flier, J. S. & Maratos-Flier, E. (2017) Beta-adrenergic receptors are critical for weight loss but not for other metabolic adaptations to the consumption of a ketogenic diet in male mice, Molecular metabolism. 6, 854-862.
67. Nakatani, T., Kuriyama, S., Tominaga, K., Tsujimoto, T., Mitoro, A., Yamazaki, M., Tsujinoue, H., Yoshiji, H., Nagao, S. & Fukui, H. (2000) Assessment of efficiency and safety of adenovirus mediated gene transfer into normal and damaged murine livers, Gut. 47, 563-70.
68. de Oliveira, M. C., Gilglioni, E. H., de Boer, B. A., Runge, J. H., de Waart, D. R., Salgueiro, C. L., Ishii-Iwamoto, E. L., Oude Elferink, R. P. & Gaemers, I. C. (2016) Bile acid receptor agonists INT747 and INT777 decrease oestrogen deficiency-related postmenopausal obesity and hepatic steatosis in mice, Biochim Biophys Acta. 1862, 2054-2062.
69. Ghebremariam, Y. T., Yamada, K., Lee, J. C., Johnson, C. L., Atzler, D., Anderssohn, M., Agrawal, R., Higgins, J. P., Patterson, A. J., Boger, R. H. & Cooke, J. P. (2013) FXR agonist INT-747 upregulates DDAH expression and enhances insulin sensitivity in high-salt fed Dahl rats, PloS one. 8, e60653.
70. Verbeke, L., Farre, R., Verbinnen, B., Covens, K., Vanuytsel, T., Verhaegen, J., Komuta, M., Roskams, T., Chatterjee, S., Annaert, P., Vander Elst, I., Windmolders, P., Trebicka, J., Nevens, F. & Laleman, W. (2015) The FXR agonist obeticholic acid prevents gut barrier dysfunction and bacterial translocation in cholestatic rats, The American journal of pathology. 185, 409-19.
71. Verbeke, L., Mannaerts, I., Schierwagen, R., Govaere, O., Klein, S., Vander Elst, I., Windmolders, P., Farre, R., Wenes, M., Mazzone, M., Nevens, F., van Grunsven, L. A., Trebicka, J. & Laleman, W. (2016) FXR agonist obeticholic acid reduces hepatic inflammation and fibrosis in a rat model of toxic cirrhosis, Scientific reports. 6, 33453.
72. Gaschott, T., Steinhilber, D., Milovic, V. & Stein, J. (2001) Tributyrin, a stable and rapidly absorbed prodrug of butyric acid, enhances antiproliferative effects of dihydroxycholecalciferol in human colon cancer cells, The Journal of nutrition. 131, 1839-43.
73. Chen, L., Chen, R., Wang, H. & Liang, F. (2015) Mechanisms Linking Inflammation to Insulin Resistance, International journal of endocrinology. 2015, 508409.
74. McArdle, M. A., Finucane, O. M., Connaughton, R. M., McMorrow, A. M. & Roche, H. M. (2013) Mechanisms of obesity-induced inflammation and insulin resistance: insights into the emerging role of nutritional strategies, Frontiers in endocrinology. 4, 52.
75. Kelly, J. R., Kennedy, P. J., Cryan, J. F., Dinan, T. G., Clarke, G. & Hyland, N. P. (2015) Breaking down the barriers: the gut microbiome, intestinal permeability and stress-related psychiatric disorders, Frontiers in cellular neuroscience. 9, 392.
76. Mudaliar, S., Henry, R. R., Sanyal, A. J., Morrow, L., Marschall, H. U., Kipnes, M., Adorini, L., Sciacca, C. I., Clopton, P., Castelloe, E., Dillon, P., Pruzanski, M. & Shapiro, D. (2013) Efficacy and safety of the farnesoid X receptor agonist obeticholic acid in patients with type 2 diabetes and nonalcoholic fatty liver disease, Gastroenterology. 145, 574-82.e1.
77. Kohli, R., Kirby, M., Xanthakos, S. A., Softic, S., Feldstein, A. E., Saxena, V., Tang, P. H., Miles, L., Miles, M. V., Balistreri, W. F., Woods, S. C. & Seeley, R. J. (2010) High-fructose, medium chain trans fat diet induces liver fibrosis and elevates plasma coenzyme Q9 in a novel murine model of obesity and nonalcoholic steatohepatitis, Hepatology (Baltimore, Md.). 52, 934-44.
78. Asgharpour, A., Cazanave, S. C., Pacana, T., Seneshaw, M., Vincent, R., Banini, B. A., Kumar, D. P., Daita, K., Min, H. K., Mirshahi, F., Bedossa, P., Sun, X., Hoshida, Y., Koduru, S. V., Contaifer, D., Jr., Warncke, U. O., Wijesinghe, D. S. & Sanyal, A. J. (2016) A diet-induced animal model of non-alcoholic fatty liver disease and hepatocellular cancer, Journal of hepatology. 65, 579-88.
79. Tahara, A., Matsuyama-Yokono, A. & Shibasaki, M. (2011) Effects of antidiabetic drugs in high-fat diet and streptozotocin-nicotinamide-induced type 2 diabetic mice, European journal of pharmacology. 655, 108-16.
80. Stephenson, K., Kennedy, L., Hargrove, L., Demieville, J., Thomson, J., Alpini, G. & Francis, H. (2017) Updates on Dietary Models of Non-Alcoholic Fatty Liver Disease: Current Studies and Insights, Gene expression.
81. Choi, H. N., Bae, J. S., Jamiyandorj, U., Noh, S. J., Park, H. S., Jang, K. Y., Chung, M. J., Kang, M. J., Lee, D. G. & Moon, W. S. (2011) Expression and role of SIRT1 in hepatocellular carcinoma, Oncology reports. 26, 503-10.
82. Zhang, X., Li, Y., Wang, D. & Wei, X. (2017) miR-22 suppresses tumorigenesis and improves radiosensitivity of breast cancer cells by targeting Sirtl, Biological research. 50, 27.
83. Lu, W., You, R., Yuan, X., Yang, T., Samuel, E. L., Marcano, D. C., Sikkema, W. K., Tour, J. M., Rodriguez, A., Kheradmand, F. & Corry, D. B. (2015) The microRNA miR-22 inhibits the histone deacetylase HDAC4 to promote T(H)17 cell-dependent emphysema, Nature immunology. 16, 1185-94.
84. Kemper, J. K., Xiao, Z., Ponugoti, B., Miao, J., Fang, S., Kanamaluru, D., Tsang, S., Wu, S. Y., Chiang, C. M. & Veenstra, T. D. (2009) FXR acetylation is normally dynamically regulated by p300 and SIRT1 but constitutively elevated in metabolic disease states, Cell Metab. 10, 392-404.
85. Xiong, F., Hu, L., Zhang, Y., Xiao, X. & Xiao, J. (2016) miR-22 inhibits mouse ovarian granulosa cell apoptosis by targeting SIRT1, Biology open. 5, 367-71.
86. Chen, H., Lu, Q., Fei, X., Shen, L., Jiang, D. & Dai, D. (2016) miR-22 inhibits the proliferation, motility, and invasion of human glioblastoma cells by directly targeting SIRT1, Tumour biology: the journal of the International Society for Oncodevelopmental Biology and Medicine. 37, 6761-8.
87. Zhang, S., Zhang, D., Yi, C., Wang, Y., Wang, H. & Wang, J. (2016) MicroRNA-22 functions as a tumor suppressor by targeting SIRT1 in renal cell carcinoma, Oncology reports. 35, 559-67.
88. Blaak, E. (2001) Gender differences in fat metabolism, Current opinion in clinical nutrition and metabolic care. 4, 499-502.
89. Geer, E. B. & Shen, W. (2009) Gender differences in insulin resistance, body composition, and energy balance, Gender medicine. 6 Suppl 1, 60-75.
90. Steinberger, J. & Daniels, S. R. (2003) Obesity, insulin resistance, diabetes, and cardiovascular risk in children: an American Heart Association scientific statement from the Atherosclerosis, Hypertension, and Obesity in the Young Committee (Council on Cardiovascular Disease in the Young) and the Diabetes Committee (Council on Nutrition, Physical Activity, and Metabolism), Circulation. 107, 1448-53.
91. Fu, Z. D., Csanaky, I. L. & Klaassen, C. D. (2012) Gender-divergent profile of bile acid homeostasis during aging of mice, PloS one. 7, e32551.
92. Zhang, L., Du, J., Yano, N., Wang, H., Zhao, Y. T., Dubielecka, P. M., Zhuang, S., Chin, Y. E., Qin, G. & Zhao, T. C. (2017) Sodium butyrate protects against high fat diet-induced cardiac dysfunction and metabolic disorders in type II diabetic mice, J. Cell Biochem. 118, 2395-2408.
93. Lin, H. V., et al. (2012) Butyrate and propionate protect against diet-induced obesity and regulate gut hormones via free fatty acid receptor 3-independent mechanisms, Plos one. 7, e35240.

### Example 3. Modulation of FGF21 Signaling by miR-22 Inhibitors and Metabolism-Enhancing Agents

This example describes a series of experiments that explored the modulation of FGF21 signaling by miR-22 inhibitors and various metabolism-enhancing agents such as retinoic acid, butyrate, and obeticholic acid (OCA).

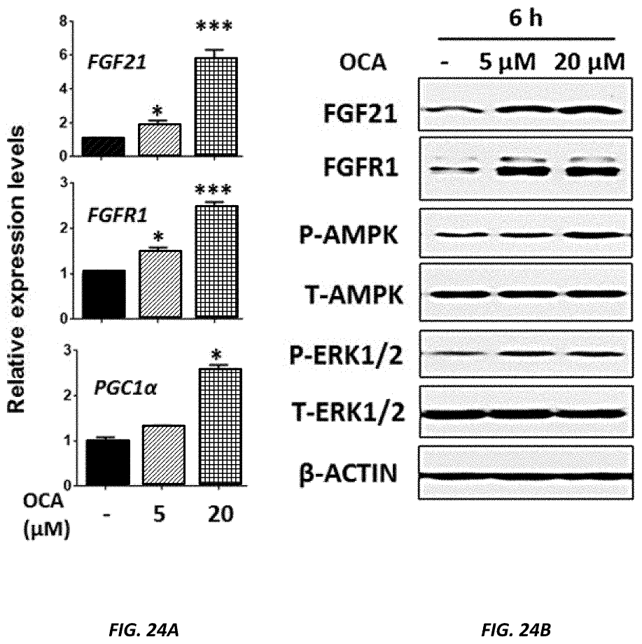

As shown in FIG. 24, OCA induced FGF21 signaling by increasing the expression of FGF21, FGF21 receptor (FGFR1), and PGC1α, which acts upstream of FGF21. mRNA levels of FGF21, FGFR1 and PGC1α (FIG. 24A) and protein levels of FGF21, FGFR1, phosphor-AMPK, total AMPK, phosphor-ERK, and total-ERK (FIG. 24B) are shown in Huh7 cells that were treated with OCA (5 μM or 20 μM) for 6 hours.

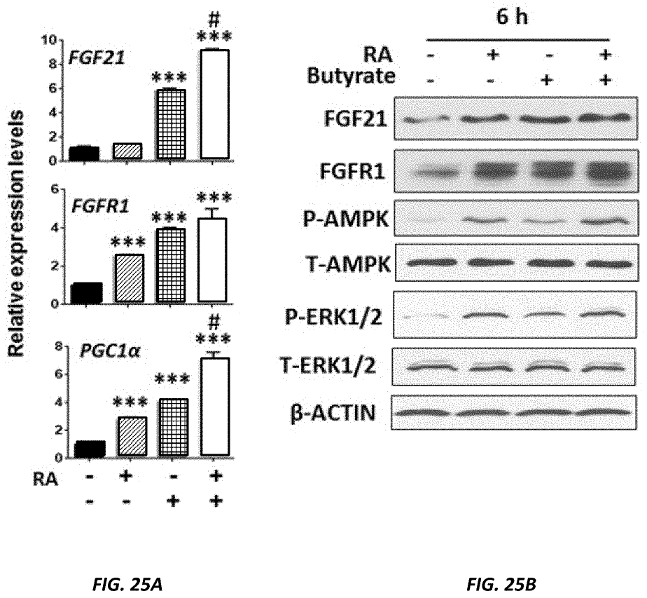

As shown in FIG. 25, retinoic acid (RA) plus butyrate induced FGF21 signaling by increasing the expression of FGF21, FGF21 receptor (FGFR1) and PGC1α, which acts upstream of FGF21. mRNA levels of FGF21, FGFR1 and PGC1α (FIG. 25A) and protein levels of FGF21, FGFR1, phosphor-AMPK, total AMPK, phosphor-ERK, and total-ERK (FIG. 25B) are shown in Huh7 cells that were treated with RA (5 butyrate (3 mM), or a combination of both for 6 hours.

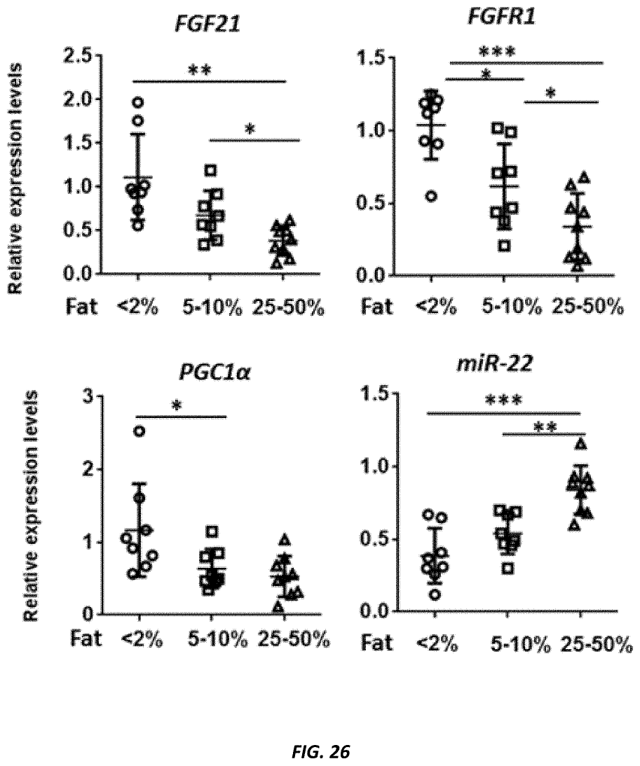

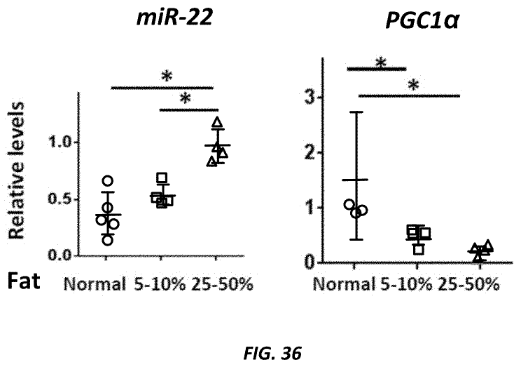

FIGS. 26 and 36 show the expression of FGF21, FGFR1, PGC1α, and miR-22 in human livers that have different fat content. Notably, FGF21 expression and miR-22 expression were inversely correlated.

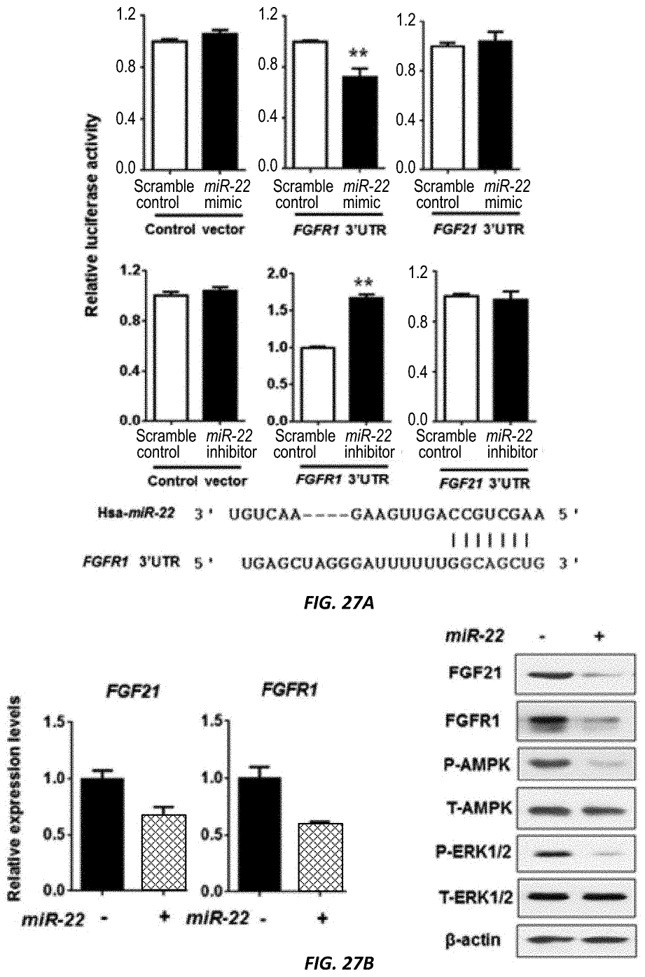

As shown in FIG. 27, FGFR1 was identified as a miR-22 direct target. In particular, miR-22 reduces both FGFR1 and FGF21. As shown in FIG. 27A, miR-22 (SEQ ID NO:2) partially pairs with the 3′UTR of the FGFR1 (SEQ ID NO:3). The miR-22 sequence is conserved between humans and mice. Adenoviral-delivery of miR-22 (adeno-miR-22-GFP) or miR-22 inhibitors (i.e., SEQ ID NO:1) tagged with green fluorescence (adeno-miR-22 inhibitor-GFP) were used to infect Huh7 cells. At 48 hours after adenoviral infection (infection efficiency >80%), psiCHECK2-FGF21 or psiCHECK2-FGFR1 constructs containing the 3′UTR of FGF21 and FGFR1 was transfected into infected Huh7 cells. Scramble constructs were used as negative controls. FIG. 27B shows the protein levels of FGF21, FGFR1, phosphor-AMPK, total AMPK, phosphor-ERK, and total-ERK in Huh7 cells that were infected with adeno-miR-22-GFP virus or empty adenovirus control after 48 hours infection.

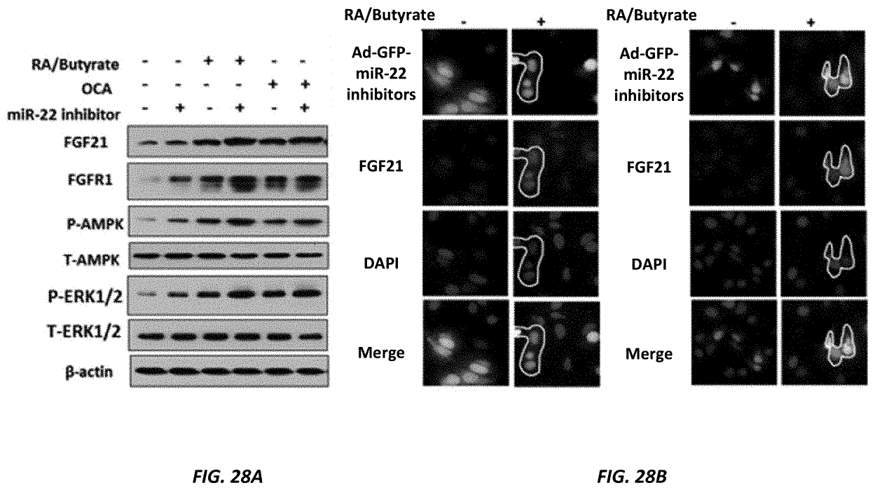

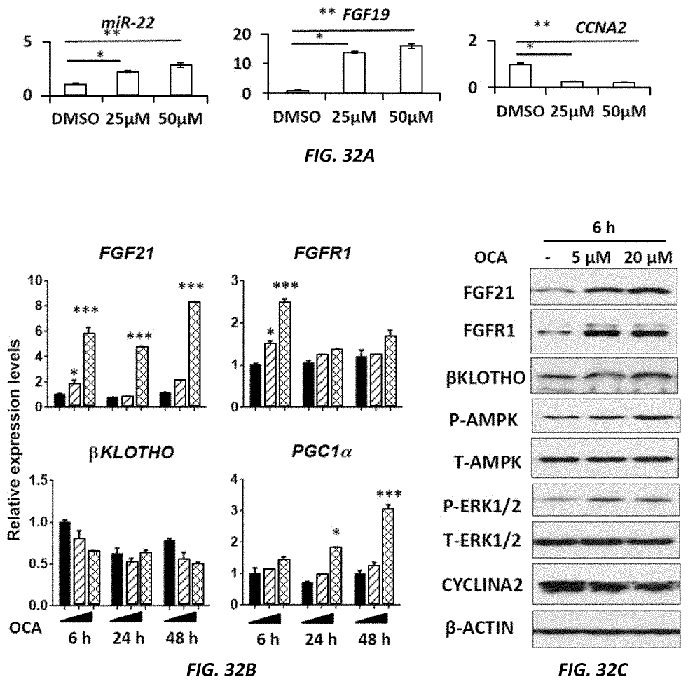

As shown in FIG. 28, miR-22 inhibition further enhances the effect of OCA and RA plus butyrate in inducing FGF21 signaling. FIG. 28A shows the protein levels of FGF21, FGFR1, phosphor-AMPK, total AMPK, phosphor-ERK, and total-ERK in Huh7 cells infected with adeno-GFP-miR-22 inhibitor or adeno-empty control followed by treatment with OCA (5 μM) or RA (5 μM) plus butyrate (3 mM) after 48 hours infection. At 48 hours post adenovirus infection, cells were treated with OCA or the combination of RA plus butyrate for 6 hours. FIG. 28B shows induction of FGF21 or FGFR1 in adeno-GFP-miR-22 infected cells followed by RA plus butyrate treatment, as indicated by immunostaining. At 48 hours post adenoviral infection, cells were treated with or without RA (5 μM) plus butyrate (3 mM) for 6 hours. Treated cells were immunostained with anti-FGF21 or anti-FGFR1 antibody followed by Alexa Fluor secondary antibody. Furthermore, OCA induced FXR target FGF19, as expected, and decreased CCNA2 expression (FIG. 32A).

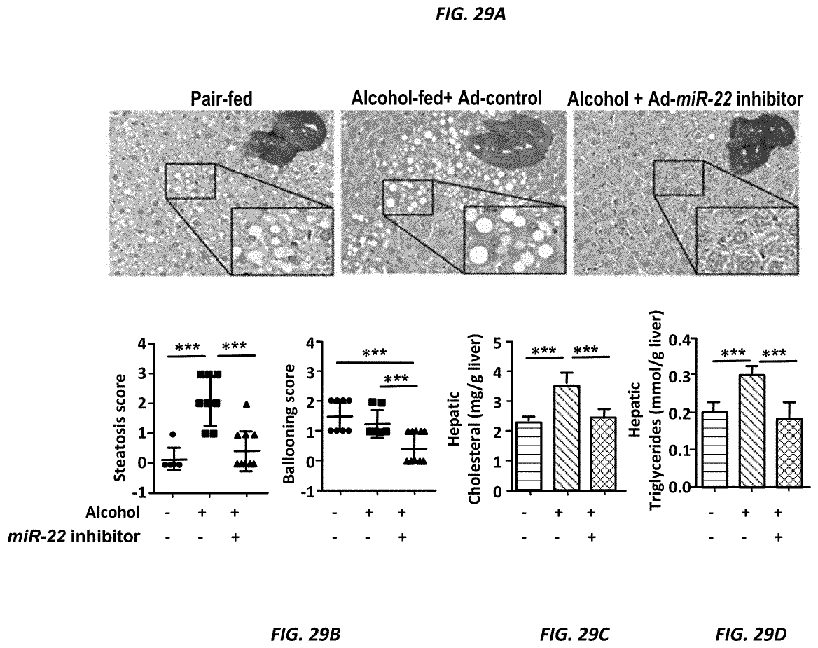

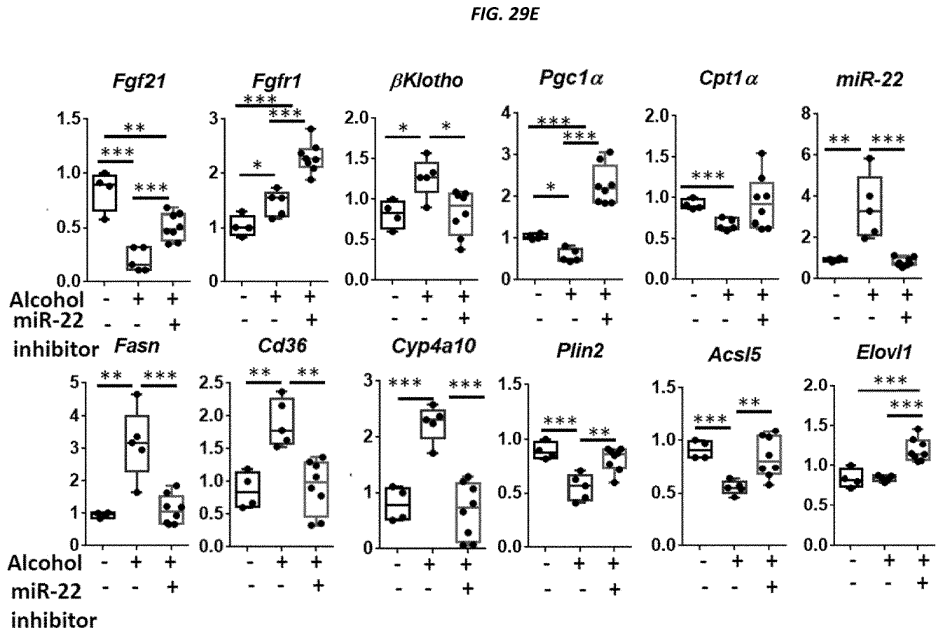

As shown in FIG. 29, adenovirus delivery of a miR-22 inhibitor rescued alcohol-induced steatosis in C57BL/6 mice. Representative H&E-stained liver sections are shown in FIG. 29A. Scores for steatosis and ballooning change are shown in FIG. 29B. Hepatic cholesterol (C) and triglycerides (D) are shown in FIGS. 29C, and 29D, respectively, as well as gene expression (FIG. 29E) in alcohol-fed mice with injection of adeno control or adeno-miR-22 inhibitor. 3-month old WT male mice were fed with Liber DeCarli diet with 5% alcohol supplementation for 3 weeks followed by adenovirus delivery of miR-22 inhibitor (1×10⁹PFU) through tail vein injection (3 times in 10 days). The empty adenovirus was used as a negative control. The mice were killed 1 day after the last injection. Steatosis scored was graded on a scale of a scale of 0 (<5%), 1 (5-33%), 2 (34-66%), and 3 (>66%). Ballooning score was graded on a scale of 0 (none), 1 (few ballooned cells), and 2 (many ballooned cells).

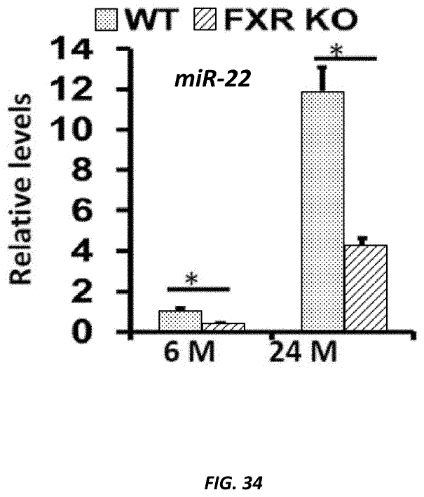

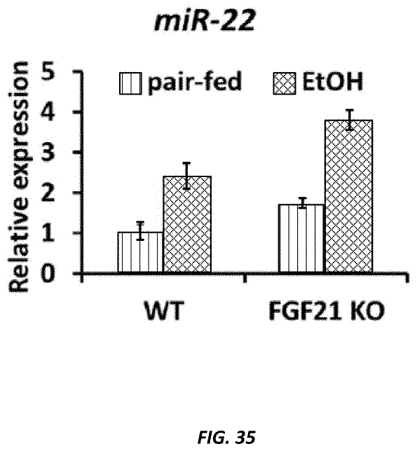

FIGS. 34 and 35 show miR-22 expression in wild-type and FGF21 KO mice. As shown in FIG. 34, whereas miR-22 is induced by bile acids, hepatic miR-22 is reduced in bile acid receptor FXR knockout mice. In addition, the expression of miR-22 was age-dependent. The aged livers (24 months old vs. 6 months old) had higher levels of miR-22, which may explain why aged livers exhibit reduced metabolism.

FIG. 35 shows miR-22 expression in C57BL/6 male wild-type mice and hepatic FGF21 KO mice that were fed with Lieber-DeCarli liquid diet for 5 days, then mice randomly divided into pair-fed and ethanol-fed (5% ethanol) groups. After 10 days, alcohol-fed mice had one dose of alcohol (5 g/kg), which was considered as a “binge.” All the mice were killed on day 12 after initiation of ethanol feeding. Using this treatment program, ethanol induced fatty livers in mice. The data showed that alcohol-induced fatty livers as well as FGF21 KO mouse livers had increased miR-22. Our data showed that hepatic FGF21 deficiency increased the susceptibility in developing fatty liver.

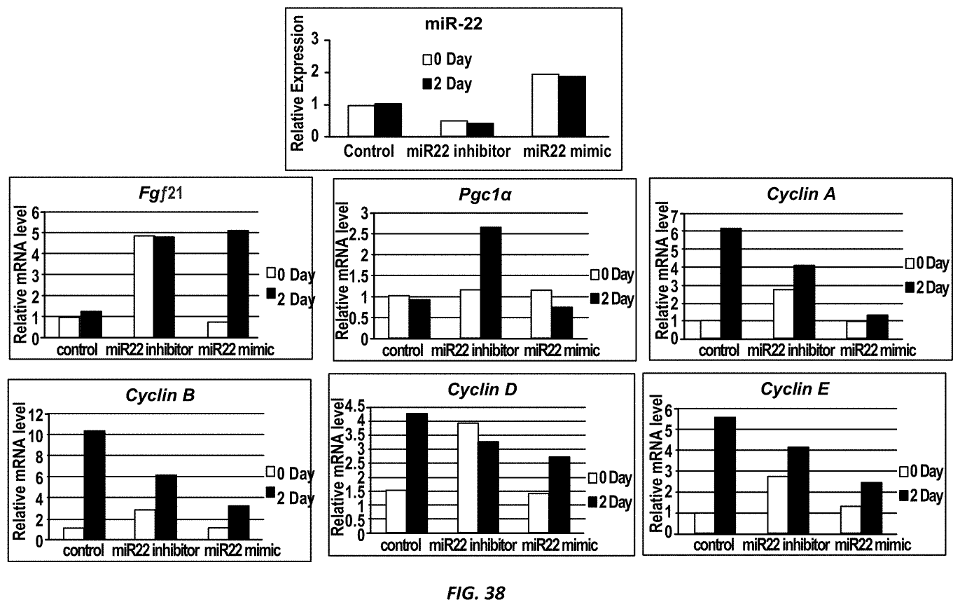

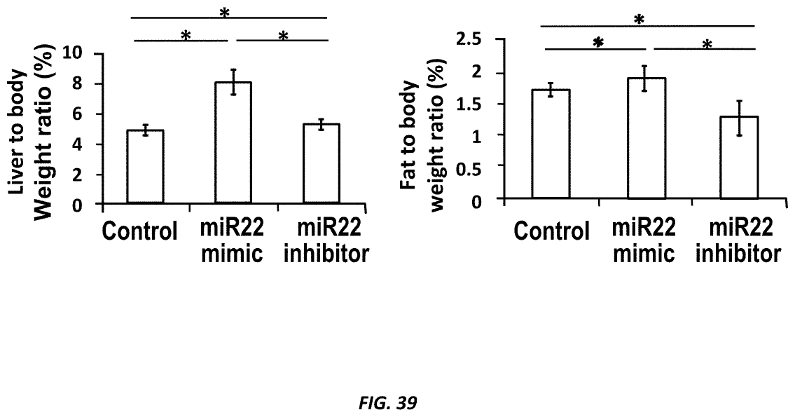

The data presented in FIGS. 38 and 39 further support the usefulness of miR-22 inhibition. For the experiments used to generate the data in FIG. 38, mice received miR-22 inhibitors or miR-22 mimics via tail vein injection 3 days prior to 2/3 liver resection (1×10⁹ pfu in 100 μL saline, one injection per day). Mice were killed on day 0 (immediately after liver resection) or 2 days later. Mice that received miR-22 inhibitors had either higher or earlier induction of FGF21, PGC1α, or Cyclin A, B, D, or E, thus indicating the beneficial effect of miR-22 inhibitors in improving metabolism-driven liver regeneration.

Furthermore, as shown in FIG. 39, the liver-to-body weight ratio and the fat-to-body weight ratio were substantially higher in miR-22 mimic-treated mice compared to other groups. Hepatic gene expression is shown in FIG. 29E. PGC1α is a master mitochondria biogenesis regulator. Carnitine palmitoyltransferase (CPT1) is a mitochondrial enzyme responsible for fatty acid metabolism. SREBP-1c regulates genes required for glucose metabolism. SREBP-1c along with fatty acid synthase (FASN) control lipid production. TNFα has a role in inflammation. miR-22 inhibitors improved metabolism by inducing PGC1α and CPT1, whereas miR-22 mimics reduced PGC1α and CPT1, but increased SREBP-1c, FASN, and TNFα.

### Example 4. Metabolic Disease Treatment by miR-22 Inhibitor-Mediated FGF21 and FGFR1 Induction

### Abstract

Background: miR-22 is a characterized tumor suppressor, but its role in regulating metabolism is unknown. Fibroblast growth factor 21 (FGF21) and its receptor FGFR1 by activating AMPK and ERK1/2 play a pivotal role in metabolism and proliferation. This example examines the linkage between miR-22 and FGF21 as well as FGFR1. The effect of miR-22 inhibitors in treating fatty liver was also studied.

Methods: Molecular biology approaches were used to elucidate the mechanism by which miR-22 silences hepatic FGF21 and FGFR1. Additionally, we studied the effect miR-22 inhibitors alone and in combination with obeticholic acid (OCA) in steatosis treatment via inducing FGF21 and FGFR1.

Results: In human and mouse fatty livers, the expression level of miR-22 and FGF21 as well as FGFR1 and PGC1α was inversely correlated. Mechanistically, miR-22 decreased FGFR1 level by direct targeting, and inhibited FGF21 expression by reduced recruitment of PPARα and PGC1α to the PPARα binding motifs located in its regulatory region. Thus, miR-22 is a metabolic silencer as well as a tumor suppressor. Adenoviral delivery of miR-22 inhibitors induced hepatic FGF21 and FGFR1 leading to AMPK and ERK1/2 activation, which was effective in treating alcoholic steatosis. In addition, miR-22 inhibitor treatment did not generate proliferative effect in the mouse liver. OCA is a semi-synthetic bile acid specific for FXR currently considered as the first drug for fatty liver. OCA was effective in inducing FGF21 and FGFR1, but it also simultaneously induced miR-22. miR-22 inhibitors, OCA, and a combination of both were all effective in treating diet-induced steatosis. Moreover, miR-22 inhibitors plus OCA generated the best effect in improving insulin sensitivity, releasing GLP-1, and reducing hepatic triglyceride in diet-induced obese mice generating metabolic disease treatment effects.

Conclusion: The induction of hepatic miR-22 likely contributes to the development of steatosis. Reducing hepatic miR-22 enhances hepatic FGF21 signaling and activates AMPK, which is effective to improve insulin sensitivity and treat steatosis. The simultaneous induction of miR-22 and FGF21 due to FXR activation may restrict FGF21-mediated ERK1/2 over-activation and thereby maintaining FGF21 homeostasis.

### Introduction

Fibroblast growth factor 21 (FGF21) is a master metabolic regulator and treatment target for metabolic diseases including type 2 diabetes mellitus (T2DM) (1-3). The action of FGF21 is mediated via its receptor FGFR1 (4-6). FGFR1 was identified as an obesity candidate gene that regulates metabolism and controls food intake (5, 7, 8). FGFR1 deficiency terminates the intracellular transduction of FGF21 signaling in adipocytes leading to reduced fatty acid oxidation and energy expenditure, indicating the critical role of FGFR1 in mediating the effect of FGF21 (5, 7). FGFR1 is reduced in both liver and white adipose tissue of obese mice, whereas FGFR1 antibody treatment ameliorated obesity and glucose intolerance in diet-induced obese mice (8-10). Mechanistically, FGF21 activates hepatic PGC1α (PPAR-activated receptor-γ coactivator-1α), a transcriptional co-activator required for fatty acid oxidation and gluconeogenic pathways, and AMPK to increase insulin sensitivity (11). ERK1/2 activation is also a downstream effect of FGF21 activation. Thus, FGF21 has a regenerative capability and can repair liver thereby linking metabolism and growth together (12, 13). Thus, it is critically important to understand the regulation FGF21 signaling in order to maintain FGF21 sensitivity and avoid over growth.

miR-22 is highly conserved across vertebrate species. We have previously characterized miR-22 as a tumor suppressor by silencing CYCLIN A2 and multiple protein deacetylases including histone deacetylase (HDAC) 1 and 4 as well as SIRT1 (14, 15). Given that miR-22 directly targets SIRT1, PGC1α, and PPARα (16-18), it would be interesting to study whether miR-22 is a metabolic silencer. Thus, the current study examined the role of miR-22 in steatosis as well as its impact on FGF21 signaling using alcohol and diet-induced steatosis mouse models.

Farnesoid X receptor (FXR) is predominantly expressed in the liver and intestine and plays an important role in regulating bile acid homeostasis, lipid and glucose metabolism, as well as inflammatory signaling (19-22). Thus, FXR agonist is a current focus of therapeutic development in metabolic disease. Obeticholic acid (OCA) is a semi-synthetic bile acid specific for FXR, which is in clinical trials for nonalcoholic steatohepatitis (NASH) (23, 24). However, OCA overdose caused deaths and liver injury. Because we previously showed that activation of FXR by bile acids induces miR-22 (14), we also studied whether silencing miR-22 has the same effect as OCA treatment.

Our data revealed for the first time that FGFR1 is a miR-22 target gene. Additionally, miR-22 inhibited FGF21 expression by reduced recruitment of PPARα and PGC1α to the PPARα binding motifs located in its regulatory region. Adenoviral delivery of miR-22 inhibitors induced hepatic FGF21 and FGFR1 leading to AMPK and ERK1/2 activation generating a treatment effect for alcoholic steatosis. In addition, miR-22 inhibitors were as effective as OCA in steatosis treatment. Moreover, miR-22 inhibitors plus OCA generated the best effect in improving insulin sensitivity and reducing hepatic triglyceride in diet-induced obese mice. Thus, miR-22 inhibitor itself is effective in treating steatosis. Moreover, miR-22 potentially can improve the efficacy of any AMPK activators.

### Materials and Methods

Cell culture: Huh7 cells were obtained from Japanese Collection of Research Bioresources and maintained in DMEM with 10% FBS. Cells were treated with DMSO, OCA (5 or 20 μM; Apexbio Technology LLC, Houston, Tex., USA) in serum-free media for indicated time. Cells were plated (1×10⁶ cells per 60-mm dish, 2×10⁵ cells per 6-well plates, and 1×10⁵ cells per 24-well plates) overnight prior to treatment or infection.

Gene expression quantification: Total RNA was extracted using TRIzol Reagent (Thermo Fisher Scientific, Waltham, Mass., USA), and cDNA was generated using High Capacity RNA-to-cDNA Kit (Applied Biosysterms, Carlsbad, Calif., USA). qRT-PCR was performed on ABI 7900HT Fast Real-time PCR system using Power SYBR® Green PCR Master Mix (Applied Biosysterms).

Constructs and luciferase reporter assay: The 3′ untranslated region (3′-UTR) of the FGFR1 gene containing the putative binding site for miR-22 and the entire 3′-UTR of the FGF21 gene (105 bp) was cloned into the psiCHECK2 vector (Promega, Madison, Wis., USA) using NotI and XhoI. For cell infection, adenovirus (negative control), adenoviral-delivered miR-22 (miR-22), or adenoviral-miR-22 inhibitors (SEQ ID NO:1; Applied Biological Materials Inc., Richmond, BC) were used. Forty-eight h post infection, cells were transfected with psiCHECK2-FGR1-3′UTR or psiCHECK2-FGF21 3′UTR using Lipofectamine 2000 (Thermo Fisher Scientific). Twenty-four h later, cells were harvested for firefly and Renilla luciferase assay using the Dual-luciferase Reporter system (Promega). Renilla luciferase activity was standardized to firefly luciferase activity.

Mice: C57BL/6 wild-type (WT) male mice (Charles River Laboratories, Inc., Wilmington, Mass., USA) were housed in regular filter-top cages at 22° C. with a 12 h/12 h light/dark cycle. For Western diet (WD)-induced obesity models, mice were fed a WD (21% fat, 34% sucrose, and 0.2% cholesterol, w/w) or control diet (CD 5% fat, 12% sucrose, and 0.01% cholesterol, w/w; Harlan Teklad, Indianapolis, Ind., USA) after weaning (3 weeks). When those mice were 7-months old, WD-fed mice were randomly assigned to 5 groups and received vehicle (0.5% carboxyl methyl cellulose sodium), OCA (10 mg/gram body weight, daily, oral gavage), miR-22 inhibitors (1×10⁹ PFU, tail vein injection, once a week), and the combination of OCA and miR-22 inhibitors for three weeks. For alcohol-induced steatosis models, 3-month-old C57BL/6 male mice were initially fed a control Lieber-DeCarli diet (F1259SP, Bio-Serv, Flemington, N.J., USA) ad libitum for 5 days followed by 5% alcohol supplementation for 21 days (F1258SP, Bio-Serv). Then, those alcohol-fed mice received adenovirus (negative control) or miR-22 inhibitors (1×10⁹ PFU, tail vein injection, 3 times in 10 days). All the mice were euthanized 1 day after the last viral injection. Animal experiments were conducted in accordance with the National Institutes of Health Guide for the Care and Use of Laboratory Animals under protocols approved by the Institutional Animal Care and Use Committee of the University of California, Davis.

Biochemical analysis: Hepatic triglyceride and cholesterol (BioAssay Systems, Hayward, Calif., USA) levels and serum lipopolysaccharide (LPS) (Thermo Fisher Scientific), alanine transaminase (ALT) (Pointe Scientific), and alkaline phosphatase (ALP) (Pointe Scientific) levels were quantified according to the manufacturer's instructions.

Insulin tolerance test: After 6 h food deprivation, tail vein blood was used to establish fasting blood glucose levels. For insulin tolerance testing, insulin (1 U/kg body weight, i.p.; MilliporeSigma, Burlington, Mass., USA) was given followed by measuring blood glucose level at various times with the OneTouch Ultra 2 (Johnson & Johnson Co., New Brunswick, N.J., USA). The area under the curve (AUC) of the blood glucose levels over time was calculated.

Serum FGF21 and glucagon-like peptide (GLP-1) secretion assay: Serum FGF21 levels were quantified by ELISA (Boster Biological Technology, Pleasanton, Calif., USA). For GLP-1 secretion assays, mice were unfed for 12 h followed by orally administration of dipeptidyl peptidase-4 inhibitor, sitagliptin (3 mg/g body weight; TSZ Chem, Framingham, Mass., USA) and a liquid diet (Ensure Plus, 10 ml/g body weight; Ross Laboratories, Columbus, Ohio, USA), which contains 15% protein, 57% carbohydrate, and 28% fat to stimulate GLP-1 secretion, as previously described (25). Blood samples were collected immediately (time 0) as well as 15, and 30 min after Ensure Plus feeding. Serum GLP-1 was quantified by a ELISA kit (RayBiotech Life, Norcross, Ga., USA).

Western blot: Western blot was performed as described previously (26). Antibodies used were anti-FGFR1, FGF21, FASN, phosphor (P)-AMPK, total (T)-AMPK, phosphor (P)-ERK1/2, and total (T)-ERK1/2 (Cell Signaling Technology, Beverly, Mass., USA), β-ACTIN (MilliporeSigma), CD36 (Bioss Antibodies Inc., Woburn, Mass., USA), CYCLIN A2, PGC1α (Novus Biologicals, LLC, Littleton, Colo., USA), and PPARα (Santa Cruz Biotechnology, Santa Cruz, Calif., USA).

Chromatin immunoprecipitation (ChIP)-qPCR: ChIP-qPCR was performed based on a previous publication (27). Briefly, chromatin lysate was precleared before incubation with anti-PGC1α (Novus Biologicals) and anti-PPARα (Santa Cruz Biotechnology). IgG (Santa Cruz Biotechnology) and RNA Polymerase II antibody (MilliporeSigma) was used as negative and positive control, respectively. Samples were incubated with Dynase beads at 4° C. overnight followed by de-crosslinking and purification. DNA fragments generated served as templates for qPCR using Power SYBR Green PCR Master Mix.

BudU immunohistochemistry: DNA synthesis was measured by immunohistochemistry for bromodeoxyuridine (BrdU) incorporation on paraffin embedded liver sections. Three-month-old male and female WT mice received adenovirus (negative control) or miR-22 inhibitors (1×10⁹PFU, tail vein injection, once a week) for 4 months. Age- and sex-matched WT mice without nay treatment were used as baseline controls. Mice were injected with 100 mg/kg BrdU (i.p., MilliporeSigma) 2 h prior to tissue harvesting. BrdU incorporation was detected by BrdU immunohistochemistry kit (Abcam, Cambridge, Mass., USA). The percentage of positive cells was determined by counting 200 nuclei in 5 fields (40X) per mouse liver.

Human livers: Hepatic fat content between 10% and 70% as well as normal livers with fat content <5% were obtained from the Gastrointestinal Biorepository at UC Davis. The tissue procurement process was approved by the UC Davis Institutional Review Board (No. 856092). Steatosis was graded by a pathologist from 0 to 3 based on fat content: grade 0 (normal)≤5%, grade 1 (mild)=5%˜33%, grade 2 (moderate)=34%˜66%, and grade 3 (severe)≥67% of hepatocytes had lipid (28).

Statistical Analysis: Data are presented as mean±SD. Statistical significance was evaluated using two-tailed Student's t-test or one-way ANOVA followed by Tukey's t-test using GraphPad Prism 6 software. A value of p<0.05 was considered statistically significant.

### Results

The Expression of miR-22 and FGF21, FGFR1, as Well as PGC1α is Inversely Correlated in the Fatty Livers

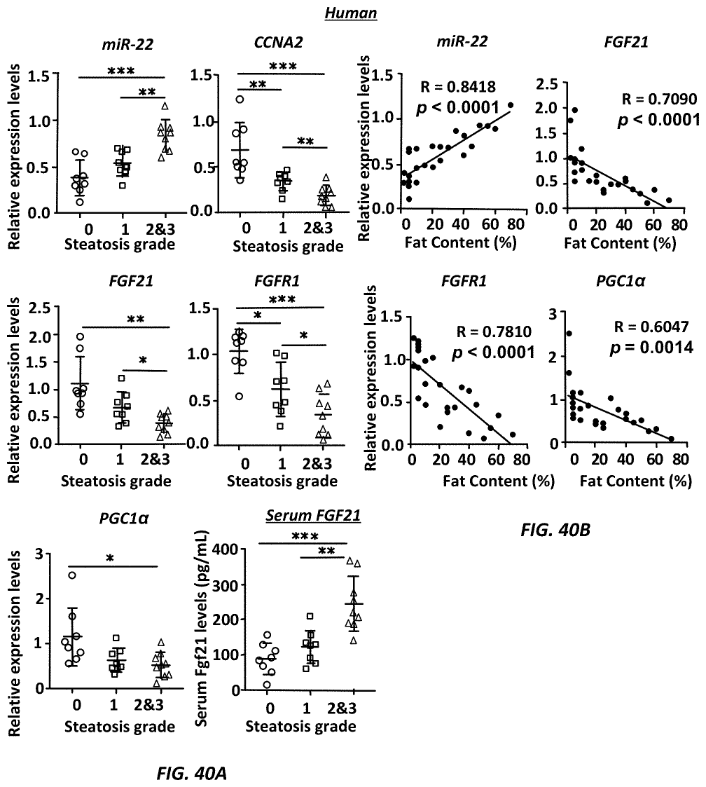

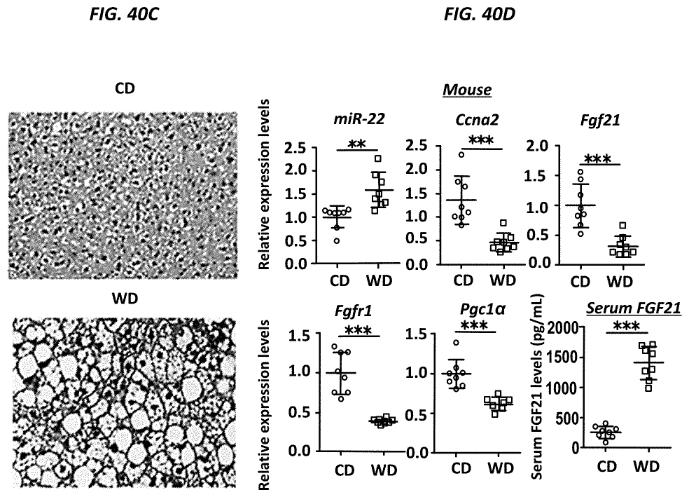

The expression levels of miR-22, FGF21, and FGFR1 as well as PGC1α were studied in human livers containing different amount of fat to reveal the potential role of miR-22 in hepatic metabolism. Hepatic miR-22 levels were significantly higher in human fatty livers in comparison with the normal livers, whereas CCNA2, a miR-22 target, was reduced in fatty livers (FIG. 40A). Furthermore, miR-22 levels were positively associated with steatosis severity (r=0.8418) (FIG. 40B). In contrast to elevated miR-22 levels, FGF21 and FGFR1 levels were progressively lower with increased hepatic fat content (FIG. 40A). Moreover, there was a significant negative relationship between the gene expression and fat content (r=0.7090 for FGF21, r=0.7810 for FGFR1) (FIG. 40B). Consistently, PGC1α, the downstream regulator of mitochondrial biogenesis, was also reduced in human fatty livers (FIGS. 40A and 40B). Similar to human data, WD-fed mice also had increased hepatic miR-22 levels but reduced Ccna2, Fgf21, Fgfr1, and Pgc1 mRNA (FIGS. 40C and 40D). In addition, serum FGF21 levels in fatty liver patients and WD-fed mice were studied since increased serum FGF21 level is considered as a biomarker for metabolic disorders and have been reported in obesity and patients with type 2 diabetes (29-31). As expected, elevated serum FGF21 level was found in both patients and obese mice, which had fatty livers (FIGS. 40A and 40C).

FGFR1 is a miR-22 Direct Target

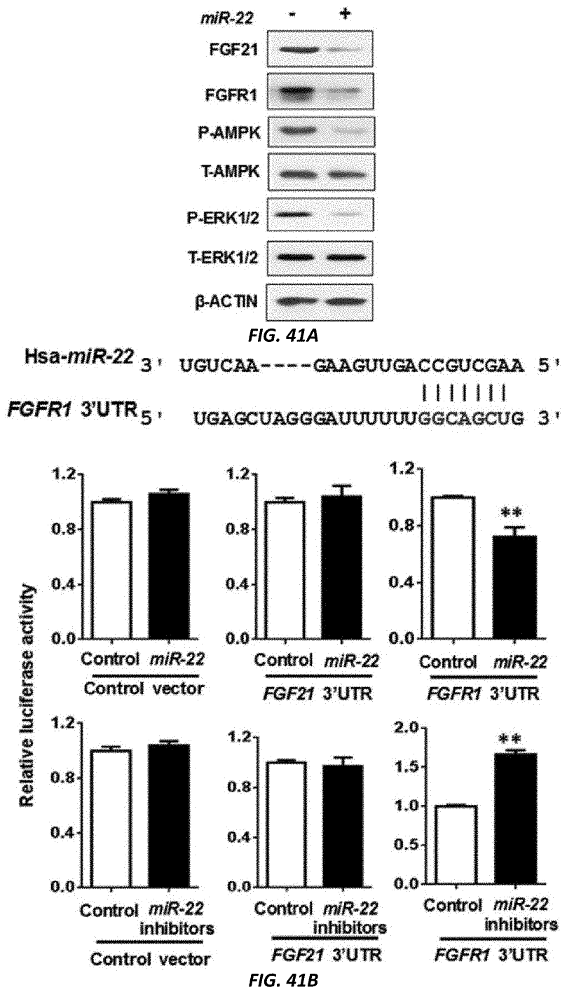

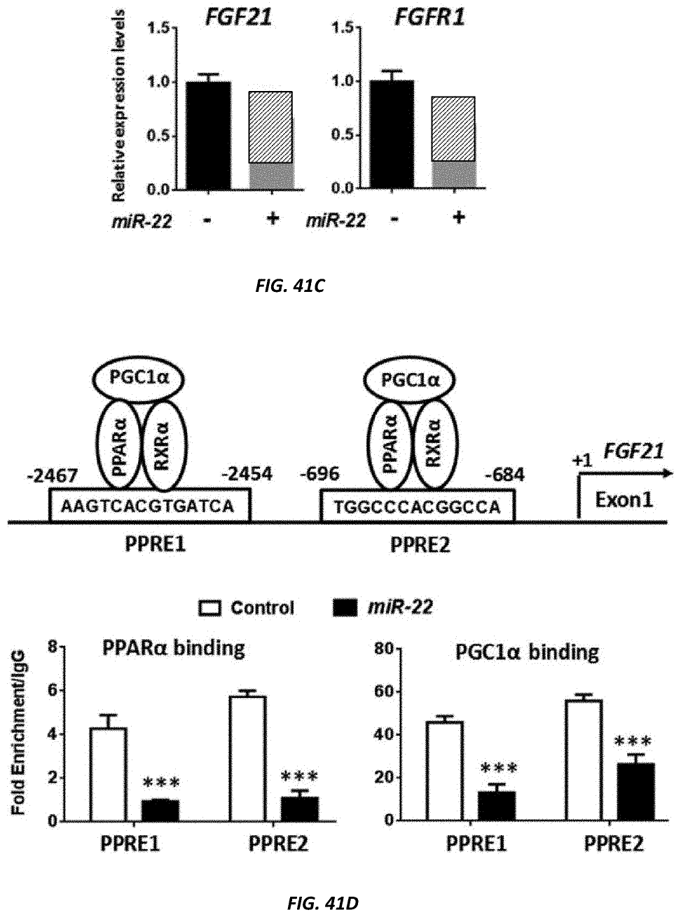

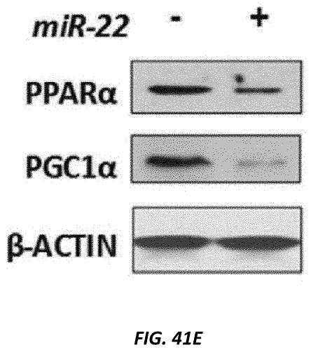

To establish the relationship between miR-22 and FGF21 signaling, Huh7 cells were infected with miR-22 for 48 h. The results showed that miR-22 infection reduced FGF21 and FGFR1 at both mRNA and protein levels, leading to AMPK and ERK1/2 deactivation without altering the level of total AMPK or ERK1/2 (FIG. 41A). Based on the ability of miR-22 to silence both FGF21 and its receptor, these findings strongly indicate that hepatic miR-22 is a metabolic silencer.

The Sanger miRBase database (http://microrna.sanger.ac.uk) predicts the presence of a highly conserved site for miR-22 to bind the 3′-UTR region of FGFR1 (FIG. 41B). In vitro function assays were performed using reporter constructs containing the miR-22 recognition sequence from the 3′UTR of FGFR1 inserted downstream of the luciferase gene. miR-22 infection decreased the luciferase activity; whereas miR-22 inhibitors increased it (FIG. 41B). To test whether miR-22 targets FGF21, the entire 3′ untranslated region (3′-UTR) of the FGF21 gene (105 bp) was cloned and assayed. The data showed that neither miR-22 nor miR-22 inhibitors changed the luciferase activity (FIG. 41B).

miR-22 Reduces FGF21 Via Decreased Recruitment of PPARα and PGC1α to the FGF21 Regulatory Region

miR-22 overexpression reduced PPARα and PGC1α in Huh7 cells, which further indicates its metabolic silencing effect (FIG. 41C). Because PPARα is a key transcriptional factor for hepatic FGF21, we tested whether inhibition of FGF21 expression by miR-22 might in part due to PPARα and PGC1α reduction. Indeed, ChIP-qPCR assay revealed that the occupancy of PPARα and PGC1α in the two peroxisome proliferative-response elements (PPREs) located in the regulatory region of the FGF21 was substantially reduced because of miR-22 overexpression in Huh7 cells (FIGS. 41D and 41E).

miR-22 Inhibitors Treat Alcoholic Steatosis

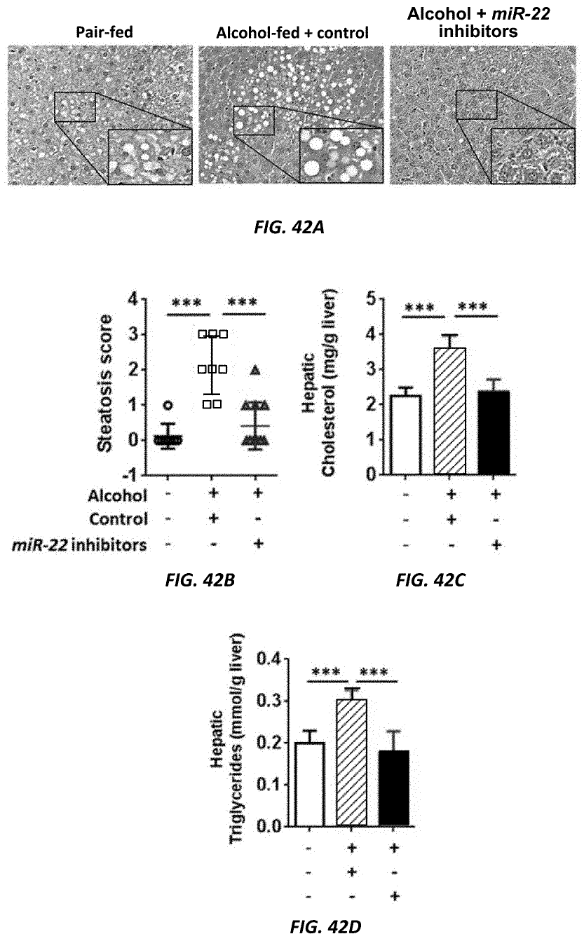

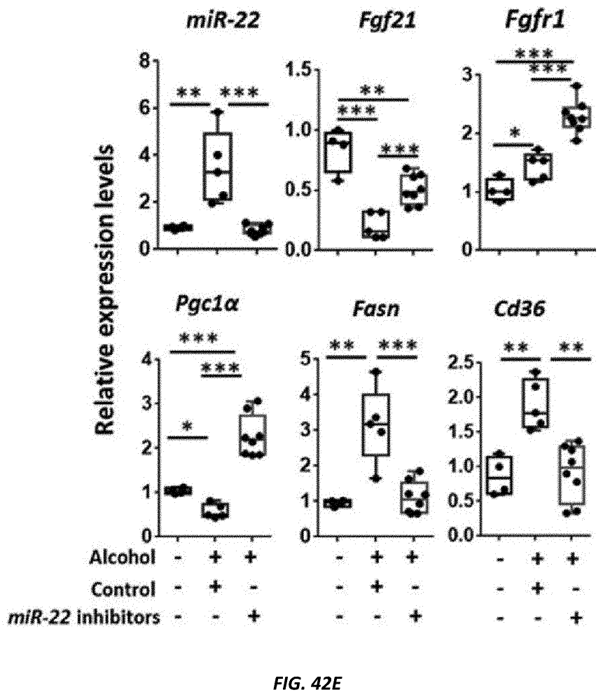

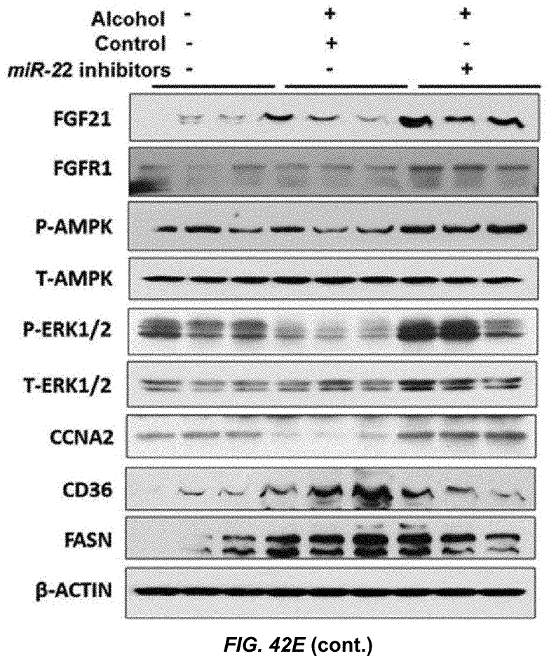

The effect of miR-22 inhibitors in treating alcoholic steatosis was studied. Morphological data showed that alcohol-fed mice developed macrovascular steatosis (FIGS. 42A and 42B). In addition, alcohol increased hepatic cholesterol and triglyceride content by about 50% compared with pair-fed control mice (FIGS. 42C and 42D). Strikingly, miR-22 inhibitors effectively eliminated alcohol-induced fat deposition and normalized hepatic cholesterol and triglyceride level (FIGS. 42A-42D), indicating the effectiveness of miR-22 inhibitors in treating steatosis.

The Effect of miR-22 Inhibitors on Hepatic FGF21 Signaling and Lipid Metabolism

In alcohol-induced fatty livers, miR-22 was induced and CCNA2 protein level was decreased. Moreover, hepatic Fgf21 and Pgc1 mRNA levels were reduced, but Fgfr1 mRNA level was modestly increased in the fatty livers. The inversed relationship between miR-22 and Fgf21 expression was consistent with the data generated in human fatty livers. Importantly, miR-22 inhibitors increased both FGF21 and FGFR1 at mRNA and protein levels leading to AMPK and ERK1/2 activation (FIG. 42E). Consistent with the histological data, miR-22 inhibitors also reduced hepatic CD36 and FASN generating beneficial treatment effect (FIG. 42E).

miR-22 Inhibitors do not have Hepatic Proliferative or Toxic Effect

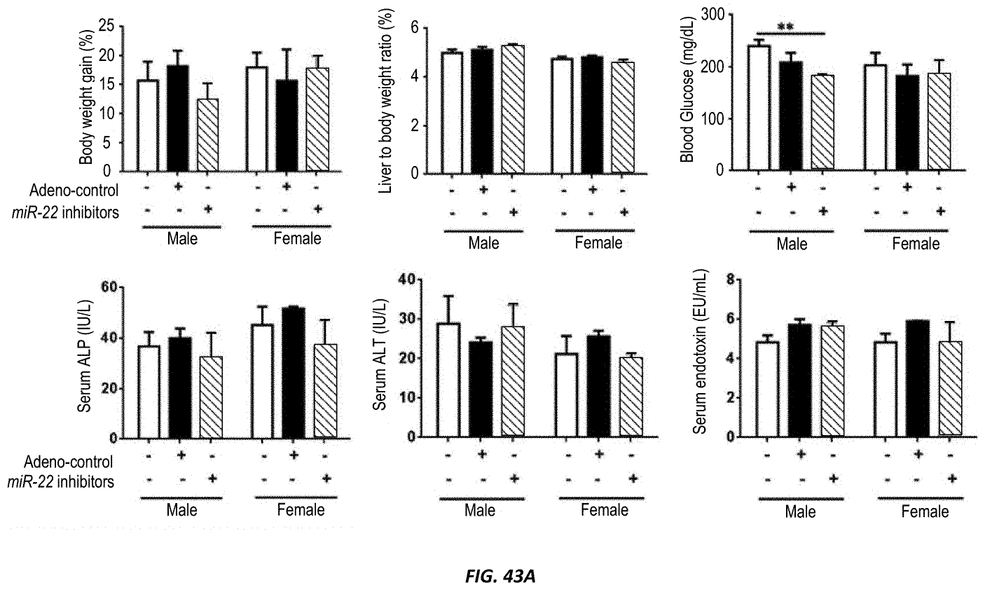

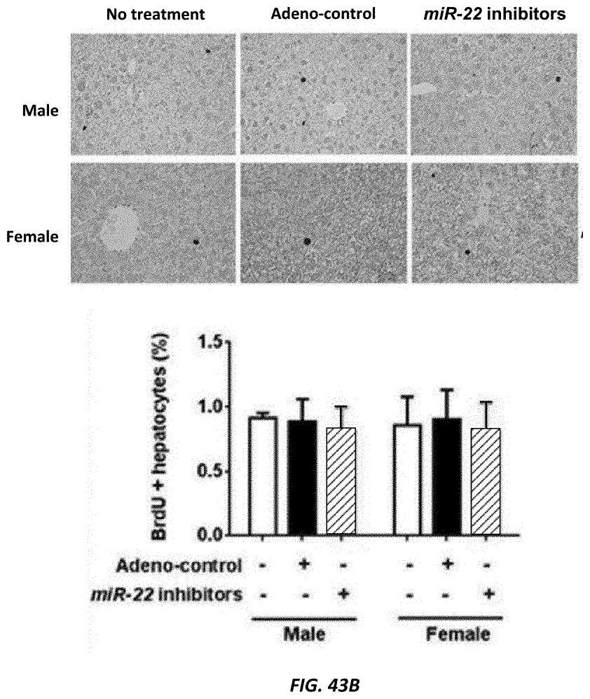

Because miR-22 expression is reduced in liver as well as colon cancer (14), we studied the potential proliferative effective of miR-22 inhibitors. After 4-months of adenovirus or miR-22 inhibitor administration, there was no difference in body weight gain or liver-to-body weight ratio between the two experimental groups (FIG. 43A). Moreover, miR-22 inhibitor delivery did not alter serum ALP, ALT, and LPS (FIG. 43A). However, miR-22 inhibitors reduced fasting blood glucose level in male mice, indicating a metabolic benefit even in a healthy condition (FIG. 43A). In addition, miR-22 inhibitors did not promote liver cell proliferation as revealed by BrdU staining (FIG. 43B). Together, miR-22 inhibitors did not generate proliferative and hepatotoxic effect.

OCA Simultaneously Induces Metabolic Silencer miR-22 as Well as Facilitators FGF21 and FGFR1

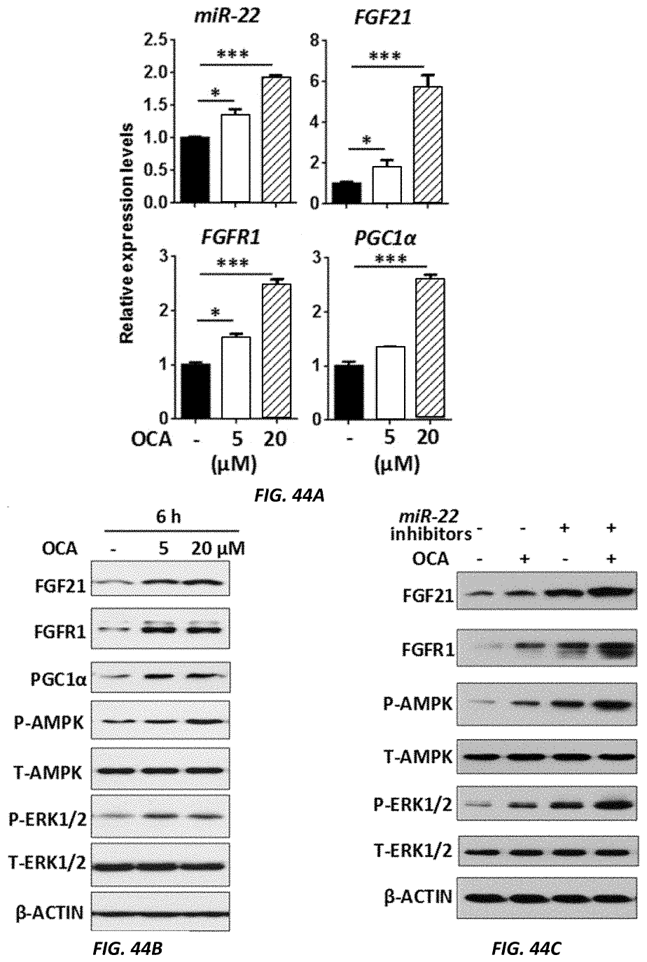

As a metabolic silencer, miR-22 is induced by FXR agonists GW6046 and chenodeoxycholic acid (14). In consistency, OCA induced miR-22 within 6 h in Huh7 cells. Moreover, such induction was accompanied by increased mRNA and protein levels of FGF21 and FGFR1 as well as PGC1α, leading to activated AMPK and ERK1/2 (FIGS. 44A and 44B).

To analyze the potential effect of OCA-induced miR-22 in regulating metabolism and growth, miR-22 inhibitors were used in conjunction with OCA treatment. Western blot data revealed miR-22 inhibitors and OCA had the same effects in increasing FGF21, FGFR1, P-AMPK, and P-ERK1/2. Additionally, when miR-22 inhibitors and OCA were used together, the levels of all those proteins were further increased compared with single reagent treatment (FIG. 44C).

The Effect of miR-22 Inhibitors and OCA in Treating Diet-Induced Steatosis

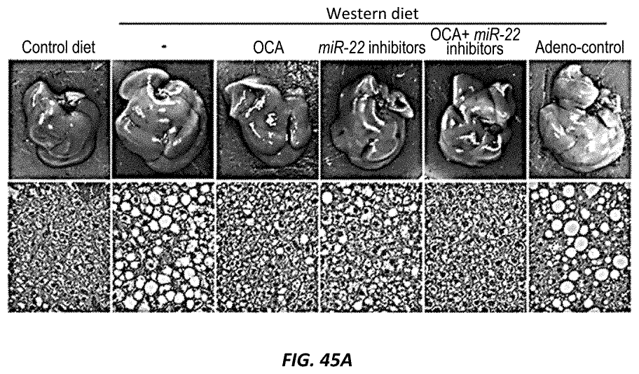

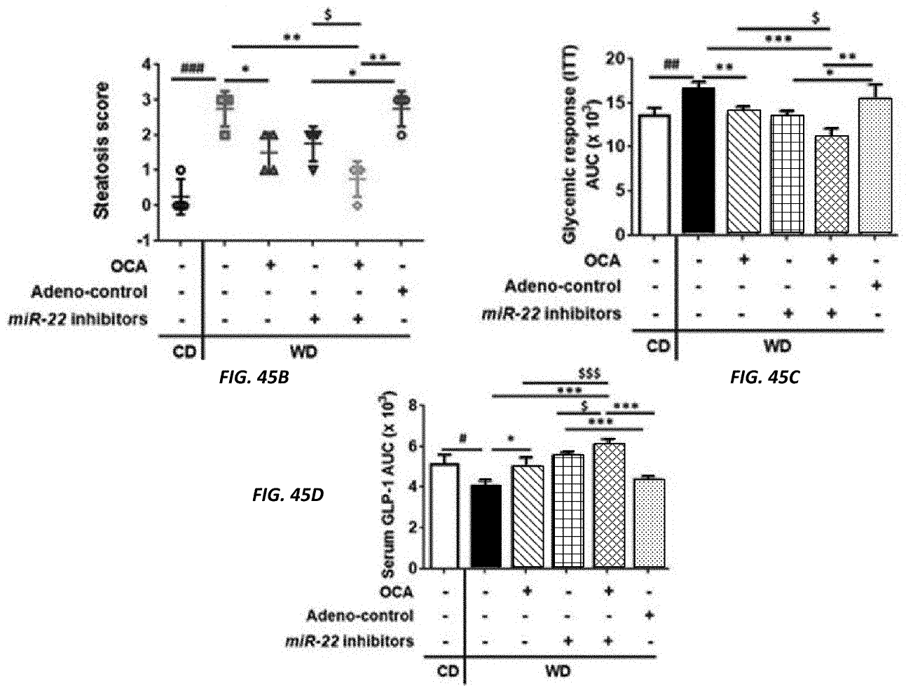

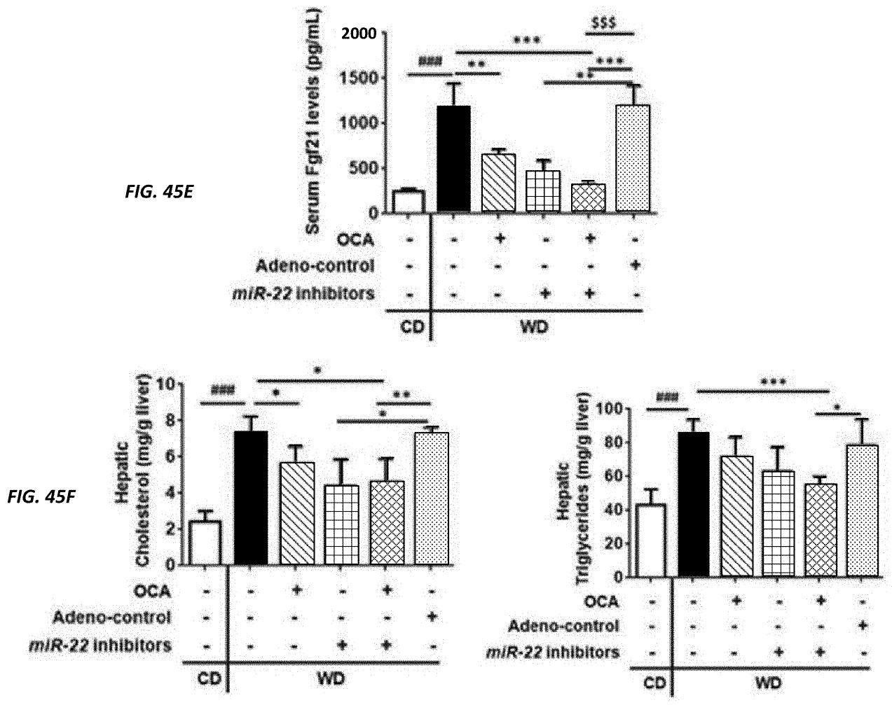

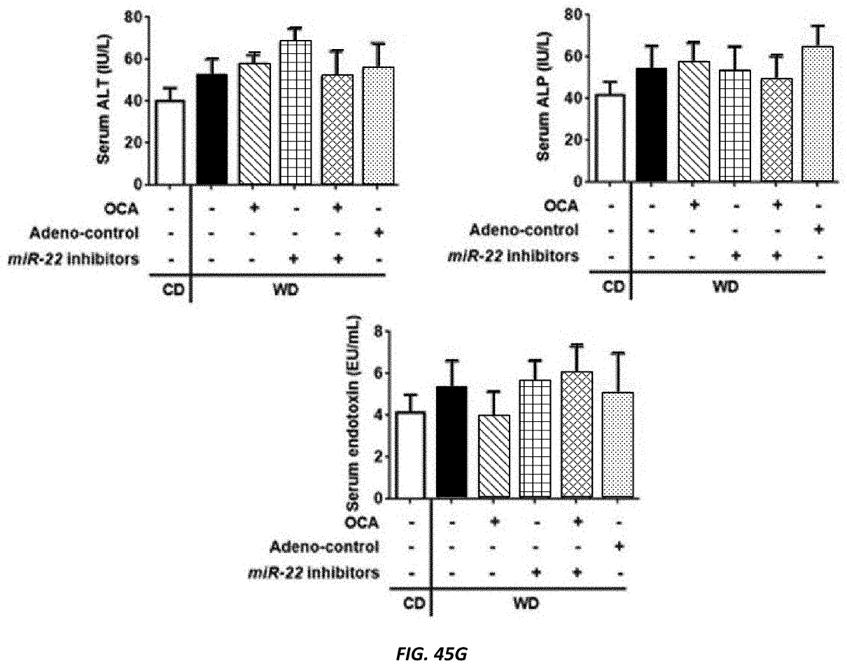

The metabolic beneficial effects miR-22 inhibitors and OCA were studied in WD-induced obese mice. Both OCA and miR-22 inhibitors reduced hepatic fat accumulation and a combination of both had better effect than single treatment (FIGS. 45A and 45B). Additionally, OCA and miR-22 inhibitors had a similar effect in normalizing insulin sensitivity and a combination treatment further improved it (FIG. 45C). OCA and miR-22 inhibitors also stimulated the release of GLP-1, which improves insulin sensitivity. A better outcome was also noted with combined treatment (FIG. 45D). Additionally, elevated serum FGF21 level in WD-fed mice was reduced by OCA and miR-22 inhibitors and the combination treatment normalized the serum FGF21 levels to the baseline shown in CD-fed mice (FIG. 45E). Further, OCA and miR-22 inhibitors reduced hepatic cholesterol level but did not significantly reduce hepatic triglyceride levels. However, combination of both significantly reduced hepatic triglycerides in WD-fed mice (FIG. 45F). Moreover, miR-22 inhibitors did not change serum ALT, ALP and LPS levels (FIG. 45G), indicating miR-22 inhibitors did not cause liver toxicity.

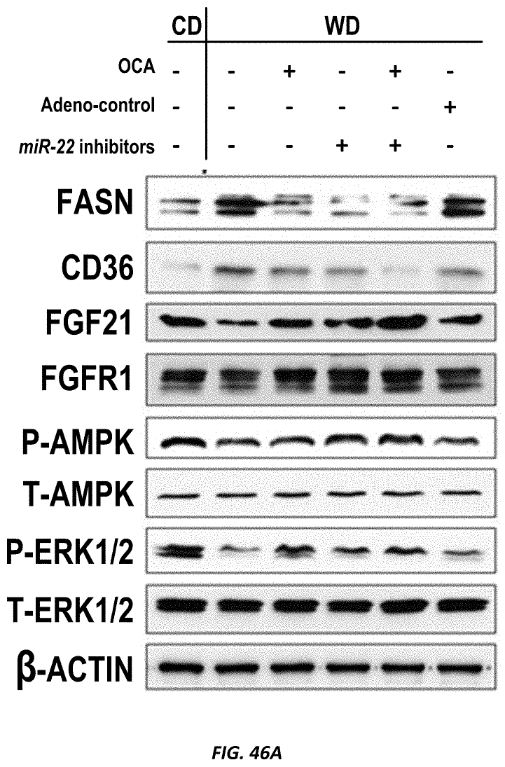

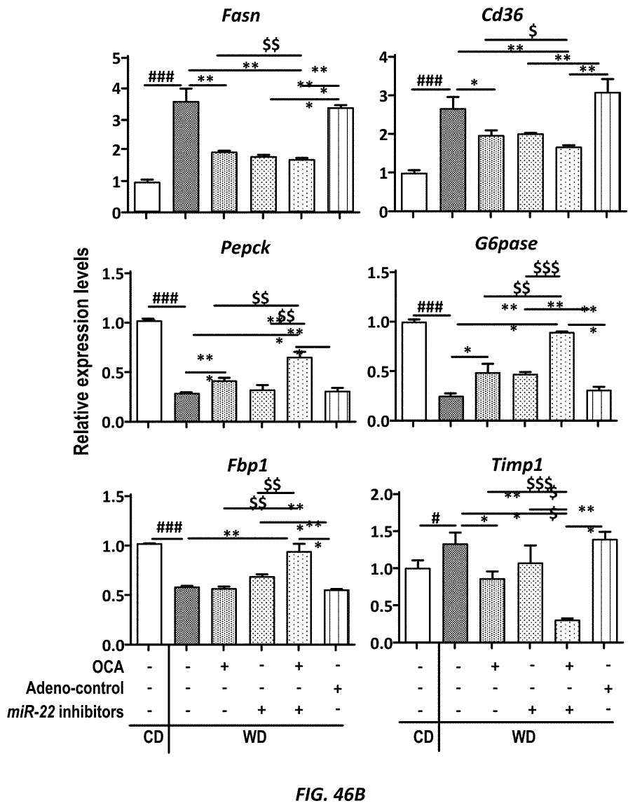

The effect of OCA and miR-22 inhibitors in regulating FGF21 signaling was also studied in diet-induced obese mice. OCA as well as miR-22 inhibitors induced hepatic FGF21 and FGFR1, which was accompanied by reduced FASN and CD36 (FIGS. 46A and 46B). The expressions of hepatic Pepck, G6pase, and Fbp1, implicated in gluconeogenesis, were remarkably reduced by WD intake, whereas OCA and/or miR22 inhibitors reversed those reductions (FIG. 46B). Additionally, it was apparent that OCA and miR-22 inhibitors together gave the best effects in reducing hepatic CD36 and Timp1 expression (FIGS. 46A and 46B).

### DISCUSSION

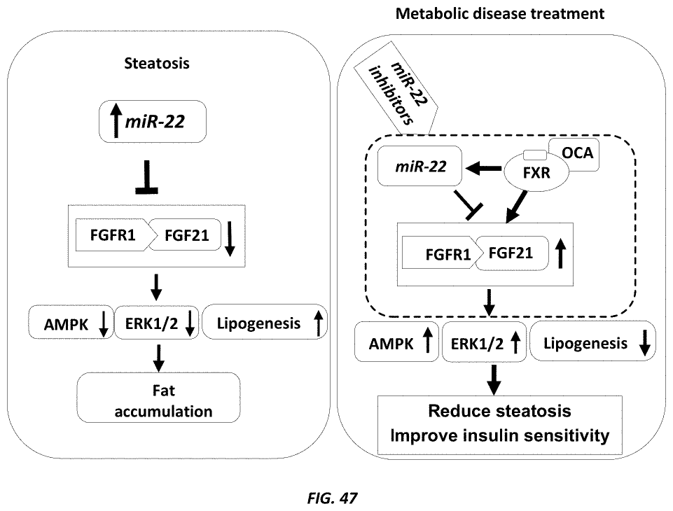

This study revealed, for the first time, that alcohol and WD intake induced miR-22; whereas blocking miR-22 expression stimulated FGF21 and FGFR1 and consequently reduced hepatic fat. Consistent with the data generated using animal models, miR-22 is highly expressed in human fatty livers. Moreover, our novel data uncovered the mechanisms by which miR-22 reduces FGF21 and FGFR1. The simultaneous reduction of both FGF21 and its receptor by m IR-22 clearly indicates the pivotal role of miR-22 in regulating metabolism. Our data also revealed that miR-22 inhibitors shared the same effects as OCA, the first drug for fatty liver treatment. Moreover, miR-22 inhibitors improve the effect of OCA. Thus, it is possible that the dose of OCA used can be reduced when miR-22 inhibitors are used in combination with OCA. The relationships between miR-22 and FGF21 signaling in fatty liver development and treatment are summarized in FIG. 47.

The use of miR-22 inhibitors combined with a FGF21 inducer OCA is an interesting therapeutic strategy to improve drug safety and efficacy of OCA. Our study revealed that OCA and miR-22 inhibitors had similar effect inducing FGF21 signaling pathway. Pathologically, combination treatment resulted in a marked reduction in hepatic CD36 expression, hepatic triglyceride level, and steatosis score. Furthermore, the combination of OCA and miR-22 inhibitors is more effective than using single treatment in improving insulin sensitivity, GLP-1 release, and reducing circulating FGF21 levels. Intestinal GLP-1 release is controlled by bile acid membrane receptor Takeda. G protein receptor (TGR)-5 (25, 32). Whether miR-22 can improve the effect of TGR5 agonist would be of interest to study.

Due to the metabolic beneficial effect of FGF21 in normalized glucose and lipid metabolism as well as energy homeostasis, FGF21 mimetics have been used in clinical trials to treat obesity, type 2 diabetes, and dyslipidemia (2, 33-35). However, the short half-life and low bioavailability of FGF21 has challenged the translational potential of FGF21 in clinical setting. Inhibition of miR-22 can be an alternative way to boost FGF21 signaling. Additionally, inhibiting miR-22 likely can improve other AMPK activators such as metformin.

Our previous data showed that miR-22 can be induced by metabolic stimulators including retinoic acid, bile acids, short-chain fatty acids such as butyrate and propionate, as well as other synthetic histone deacetylase inhibitors like suberanilohydroxamic acid that is used to combat cancer (14, 15). In addition, the induction of miR-22 is dependent on nuclear receptor RARβ and FXR, which are both tumor suppressors (14, 15). Since miR-22 is a tumor suppressor as well as metabolic silencer, the induction of miR-22 by those metabolic facilitators likely is to restrict metabolism-driven over growth regulated by ERK1/2. It is also possible that such a negative feedback mechanism controlled by miR-22 avoids consistent FGF21 induction thereby leading to FGF21 homeostasis and sensitivity to insulin.

As mentioned above, the expression of miR-22 is transcriptionally regulated by nuclear receptors and epigenetically regulated by inhibiting histone deacetylation. Because the transcriptional activity of FXR and RARβ is inhibited in metabolically compromised conditions, the mechanism by which miR-22 is induced in fatty liver remains to be elucidated. Our previous data showed miR-22 itself has a broad HDAC inhibitory effect by directly targeting HDAC1, HDAC4, and SIRT1 (15). Because HDAC can modify nuclear receptor acetylation in addition to histone modification (36), there is a possibility that miR-22-reduced protein deacetylases can modify nuclear receptors and affect their transcriptional activity.

During the progression of liver disease from steatosis to liver cancer formation, miR-22 is induced followed by decline. In the steatosis phase, increased miR-22 slows down hepatic metabolism and has a growth inhibitory effect; whereas in the carcinogenesis phase, the reduced miR-22 facilitates metabolism and supports growth. Thus, the expression of miR-22 needs to be fine-tuned in order to maintain balanced metabolism and growth. miR-22 inhibitors and mimics are capable of adjusting the over- and under-expression conditions without inducing unwanted effects as evidenced by the lack of a proliferative effect when miR-22 inhibitors were used. Together, hepatic miR-22 inhibition to increase FGF21 activation is an attractive and novel therapeutic approach for treating metabolic diseases.

### REFERENCES

1. Degirolamo C, Sabba C, Moschetta A. Therapeutic potential of the endocrine fibroblast growth factors FGF19, FGF21 and FGF23. Nat Rev Drug Discov 2016; 15:51-69.
2. Woo Y C, Xu A M, Wang Y, Lam KSL. Fibroblast Growth Factor 21 as an emerging metabolic regulator: clinical perspectives. Clinical Endocrinology 2013; 78:489-496.
3. So W Y, Leung P S. Fibroblast Growth Factor 21 As an Emerging Therapeutic Target for Type 2 Diabetes Mellitus. Medicinal Research Reviews 2016; 36:672-704.
4. Yang C, Jin C, Li X, Wang F, McKeehan W L, Luo Y. Differential specificity of endocrine FGF19 and FGF21 to FGFR1 and FGFR4 in complex with KLB. PLoS One 2012; 7:e33870.
5. Adams A C, Yang C, Coskun T, Cheng C C, Gimeno R E, Luo Y, Kharitonenkov A. The breadth of FGF21's metabolic actions are governed by FGFR1 in adipose tissue. Mol Metab 2012; 2:31-37.
6. Foltz I N, Hu S, King C, Wu X, Yang C, Wang W, Weiszmann J, et al. Treating diabetes and obesity with an FGF21-mimetic antibody activating the betaKlotho/FGFR1c receptor complex. Sci Transl Med 2012; 4:162ra153.
7. Ye M, Lu W, Wang X, Wang C, Abbruzzese J L, Liang G, Li X, et al. FGF21-FGFR1 Coordinates Phospholipid Homeostasis, Lipid Droplet Function, and E R Stress in Obesity. Endocrinology 2016; 157:4754-4769.
8. Fisher F M, Chui P C, Antonellis P J, Bina H A, Kharitonenkov A, Flier J S, Maratos-Flier E. Obesity is a fibroblast growth factor 21 (FGF21)-resistant state. Diabetes 2010; 59:2781-2789.
9. Lelliott C J, Ahnmark A, Admyre T, Ahlstedt I, Irving L, Keyes F, Patterson L, et al. Monoclonal antibody targeting of fibroblast growth factor receptor 1c ameliorates obesity and glucose intolerance via central mechanisms. PLoS One 2014; 9:e112109.
10. Wu A L, Kolumam G, Stawicki S, Chen Y, Li J, Zavala-Solorio J, Phamluong K, et al. Amelioration of type 2 diabetes by antibody-mediated activation of fibroblast growth factor receptor 1. Sci Transl Med 2011; 3:113ra126.
11. Potthoff M J, Inagaki T, Satapati S, Ding X, He T, Goetz R, Mohammadi M, et al. FGF21 induces PGC-1alpha and regulates carbohydrate and fatty acid metabolism during the adaptive starvation response. Proc Natl Acad Sci USA 2009; 106:10853-10858.
12. Liu H X, Hu Y, Wan Y J Y. Microbiota and bile acid profiles in retinoic acid-primed mice that exhibit accelerated liver regeneration. Oncotarget 2016; 7:1096-1106.
13. Liu H X, Hu Y, French S W, Gonzalez F J, Wan Y J. Forced expression of fibroblast growth factor 21 reverses the sustained impairment of liver regeneration in hPPARalpha(PAC) mice due to dysregulated bile acid synthesis. Oncotarget 2015; 6:9686-9700.
14. Yang F, Hu Y, Liu H X, Wan Y J Y. MiR-22-silenced Cyclin A Expression in Colon and Liver Cancer Cells Is Regulated by Bile Acid Receptor. Journal of Biological Chemistry 2015; 290:6507-6515.
15. Hu Y, French S W, Chau T, Liu H X, Sheng L, Wei F, Stondell J, et al. RARbeta acts as both an upstream regulator and downstream effector of miR-22, which epigenetically regulates NUR77 to induce apoptosis of colon cancer cells. FASEB J 2018:fj201801390R.
16. Gurha P, Wang T, Larimore A H, Sassi Y, Abreu-Goodger C, Ramirez M O, Reddy A K, et al. microRNA-22 promotes heart failure through coordinate suppression of PPAR/ERR-nuclear hormone receptor transcription. PLoS One 2013; 8:e75882.
17. Iliopoulos D, Malizos K N, Oikonomou P, Tsezou A. Integrative microRNA and proteomic approaches identify novel osteoarthritis genes and their collaborative metabolic and inflammatory networks. PLoS One 2008; 3:e3740.
18. Du J K, Cong B H, Yu Q, Wang H, Wang L, Wang C N, Tang X L, et al. Upregulation of microRNA-22 contributes to myocardial ischemia-reperfusion injury by interfering with the mitochondrial function. Free Radic Biol Med 2016; 96:406-417.
19. Kuipers F, Bloks V W, Groen A K. Beyond intestinal soap—bile acids in metabolic control. Nat Rev Endocrinol 2014; 10:488-498.
20. Chiang J Y. Bile acid metabolism and signaling. Compr Physiol 2013; 3:1191-1212.
21. Stayrook K R, Bramlett K S, Savkur R S, Ficorilli J, Cook T, Christe M E, Michael L F, et al. Regulation of carbohydrate metabolism by the farnesoid X receptor. Endocrinology 2005; 146:984-991.
22. Bilz S, Samuel V, Morino K, Savage D, Choi C S, Shulman G I. Activation of the farnesoid X receptor improves lipid metabolism in combined hyperlipidemic hamsters. Am J Physiol Endocrinol Metab 2006; 290:E716-722.
23. Neuschwander-Tetri B A, Loomba R, Sanyal A J, Lavine J E, Van Natta M L, Abdelmalek M F, Chalasani N, et al. Farnesoid X nuclear receptor ligand obeticholic acid for non-cirrhotic, non-alcoholic steatohepatitis (FLINT): a multicentre, randomised, placebo-controlled trial. Lancet 2015; 385:956-965.
24. Thomas C, Pellicciari R, Pruzanski M, Auwerx J, Schoonjans K. Targeting bile-acid signalling for metabolic diseases. Nat Rev Drug Discov 2008; 7:678-693.
25. Sheng L, Jena P K, Liu H X, Hu Y, Nagar N, Bronner D N, Settles M L, et al. Obesity treatment by epigallocatechin-3-gallate-regulated bile acid signaling and its enriched Akkermansia muciniphila. FASEB J 2018:fj201800370R.
26. Yang H, Zhan Q, Wan Y J. Enrichment of Nur77 mediated by retinoic acid receptor beta leads to apoptosis of human hepatocellular carcinoma cells induced by fenretinide and histone deacetylase inhibitors. Hepatology 2011; 53:865-874.
27. Hu Y, Liu H X, He Y, Fang Y, Fang J, Wan Y J. Transcriptome profiling and genome-wide DNA binding define the differential role of fenretinide and all-trans R A in regulating the death and survival of human hepatocellular carcinoma Huh7 cells. Biochem Pharmacol 2013; 85:1007-1017.
28. Kleiner D E, Brunt E M, Van Natta M, Behling C, Contos M J, Cummings O W, Ferrell L D, et al. Design and validation of a histological scoring system for nonalcoholic fatty liver disease. Hepatology 2005; 41:1313-1321.
29. Zhang X, Yeung D C, Karpisek M, Stejskal D, Zhou Z G, Liu F, Wong R L, et al. Serum FGF21 levels are increased in obesity and are independently associated with the metabolic syndrome in humans. Diabetes 2008; 57:1246-1253.
30. Xiao Y, Liu L, Xu A, Zhou P, Long Z, Tu Y, Chen X, et al. Serum fibroblast growth factor 21 levels are related to subclinical atherosclerosis in patients with type 2 diabetes. Cardiovasc Diabetol 2015; 14:72.
31. Tanajak P, Pongkan W, Chattipakorn S C, Chattipakorn N. Increased plasma FGF21 level as an early biomarker for insulin resistance and metabolic disturbance in obese insulin-resistant rats. Diab Vasc Dis Res 2018; 15:263-269.
32. Pathak P, Liu H, Boehme S, Xie C, Krausz K W, Gonzalez F, Chiang J Y L. Farnesoid X receptor induces Takeda G-protein receptor 5 cross-talk to regulate bile acid synthesis and hepatic metabolism. J Biol Chem 2017; 292:11055-11069.
33. Owen B M, Mangelsdorf D J, Kliewer S A. Tissue-specific actions of the metabolic hormones FGF15/19 and FGF21. Trends Endocrinol Metab 2015; 26:22-29.
34. Kharitonenkov A, Adams A C. Inventing new medicines: The FGF21 story. Mol Metab 2014; 3:221-229.
35. Gaich G, Chien J Y, Fu H D, Glass L C, Deeg M A, Holland W L, Kharitonenkov A, et al. The Effects of LY2405319, an FGF21 Analog, in Obese Human Subjects with Type 2 Diabetes. Cell Metabolism 2013; 18:333-340.
36. Wang C, Tian L, Popov V M, Pestell R G. Acetylation and nuclear receptor action. J Steroid Biochem Mol Biol 2011; 123:91-100.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Informal Sequence Listing

| SEQ ID NO: | Sequence | Description |
|---|---|---|
| 1 | 5′-ACAGTTCTTCAACTGGCAGCTT-3′ | hsa-miR-22-3p inhibitor |
| | | sequence |
| 2 | 5′-AAGCUGCCAGUUGAAGAACUGU-3′ | hsa-miR-22-3p sequence |
| 3 | 5′-UGAGCUAGGGAUUUUUUGGCAGCUG-3′ | FGFR1 3′ UTR |

1. A method for preventing or treating a metabolic disease in a subject, the method comprising administering to the subject a therapeutically effective amount of a microRNA-22 (miR-22) inhibitor.

2. The method of claim 1, wherein the miR-22 inhibitor is an inhibitor of human miR-22 (hsa-miR-22).

3. The method of claim 1, wherein the miR-22 inhibitor is an oligonucleotide.

4. The method of claim 3, wherein the oligonucleotide comprises a nucleic acid sequence that hybridizes to miR-22 and reduces miR-22 expression.

5. The method of claim 4, wherein the oligonucleotide comprises a nucleic acid sequence that has at least about 80% sequence identity to SEQ ID NO:1.

6. (canceled)

7. The method of claim 3, wherein the oligonucleotide is expressed from an adenovirus, an adeno-associated virus (AAV), a lentivirus, or other delivery system.

8. The method of claim 1, further comprising administering to the subject a therapeutically effective amount of a metabolism-enhancing agent.

9. (canceled)

10. The method of claim 8, wherein the metabolism-enhancing agent is selected from the group consisting of fibroblast growth factor 21 (FGF21), an FGF21 mimic that increases FGF21 activity, a retinoid, a histone deacetylase (HDAC) inhibitor, metformin, a bile acid or an analog thereof, a bile acid receptor agonist, a resistant starch, a prebiotic agent, a probiotic agent, and a combination thereof.

11-17. (canceled)

18. The method of claim 1, further comprising administering to the subject a delivery-enhancing agent.

19-22. (canceled)

23. The method of claim 1, wherein the metabolic disease is selected from the group consisting of alcoholic steatohepatitis (ASH), non-alcoholic steatohepatitis (NASH), non-alcoholic fatty liver disease (NAFLD), diabetes, obesity, dyslipidemia, and a combination thereof.

24-39. (canceled)

40. A pharmaceutical composition comprising a microRNA-22 (miR-22) inhibitor, a metabolism-enhancing agent, and a pharmaceutically acceptable carrier.

41. The pharmaceutical composition of claim 40, wherein the miR-22 inhibitor is an inhibitor of human miR-22 (hsa-miR-22).

42. The pharmaceutical composition of claim 40, wherein the miR-22 inhibitor is an oligonucleotide.

43. The pharmaceutical composition of claim 42, wherein the oligonucleotide comprises a nucleic acid sequence that has at least about 80% sequence identity to SEQ ID NO:1.

44. The pharmaceutical composition of claim 40, wherein the metabolism-enhancing agent is selected from the group consisting of fibroblast growth factor 21 (FGF21), an FGF21 mimic that increases FGF21 activity, a retinoid, a histone deacetylase (HDAC) inhibitor, metformin, a bile acid or an analog thereof, a resistant starch, a prebiotic agent, a probiotic agent, and a combination thereof.

45-54. (canceled)

55. The pharmaceutical composition of claim 40, wherein the pharmaceutical composition comprises a therapeutically effective amount of the miR-22 inhibitor and/or the metabolism-enhancing agent.

56. The pharmaceutical composition of claim 40, further comprising a delivery-enhancing agent.

57. The pharmaceutical composition of claim 56, wherein the delivery-enhancing agent is selected from the group consisting of a cyclodextrin, an inactivated bacterium, polyvinyl alcohol (PVA), an inulin or an ester thereof, and a combination thereof.

58-60. (canceled)

61. A kit for preventing or treating a metabolic disease in a subject, the kit comprising the pharmaceutical composition of claim 40.

62. The kit of claim 61, wherein the metabolic disease is selected from the group consisting of alcoholic steatohepatitis (ASH), non-alcoholic steatohepatitis (NASH), non-alcoholic fatty liver disease (NAFLD), diabetes, obesity, dyslipidemia, and a combination thereof.

63. (canceled)


SEQUENCE LISTING (5 sequences)

SEQ ID NO: 1 - DNA, 22 bp, Homo sapiens
acagttcttcaactggcagctt

SEQ ID NO: 2 - RNA, 22 nt, Homo sapiens
aagcugccaguugaagaacugu

SEQ ID NO: 3 - RNA, 25 nt, Unknown
  Description of Unknown FGFR1 3′ UTR
ugagcuagggauuuuuuggcagcug

SEQ ID NO: 4 - DNA, 14 bp, Unknown
  Description of Unknown peroxisome proliferative-response element sequence
aagtcacgtgatca

SEQ ID NO: 5 - DNA, 13 bp, Unknown
  Description of Unknown peroxisome proliferative-response element sequence
tggcccacggcca